SEQUENCE LISTING (756 sequences)

SEQ ID NO: 1 - DNA, 122 bp, synthetic construct
  source 1..122: mol_type = other DNA
aaagctaacaattggcttggagggtaaaagaccactgcagttcaccctccctgagctggacgtacaatttgtccagtgttaggaaatgtccggcttagcagtgagaggcttgaagacacccc

SEQ ID NO: 2 - DNA, 122 bp, synthetic construct
  source 1..122: mol_type = other DNA
cttggcccattagctggggagctgacaaatcaccaggggcgctgagccgcacacaccactcggctttaaagaagcgatggggtcgttaccagccgattatccagacacacaatcactcacag

SEQ ID NO: 3 - DNA, 122 bp, synthetic construct
  source 1..122: mol_type = other DNA
gagacggcgttaggaatctcactggaaggaggggtgtcttcaagcctctcactgctaagccggacatttcctaacactggacaaattgtacgtccagctcagggagggtgaactgcagtggt

SEQ ID NO: 4 - DNA, 122 bp, synthetic construct
  source 1..122: mol_type = other DNA
ccaggctccgctgaggtttctcgggtgaaacacgcctctgtgatggctgtgtctgtaaagcggatggaaatgcccataaagccgtatcaaaccctgtgcagagaaggacagggatatatata

SEQ ID NO: 5 - DNA, 122 bp, synthetic construct
  source 1..122: mol_type = other DNA
ttggatcaatccgtccgcccgccgctccctcctccccctcctccctccctccgcctaatgcgctttatcaggagacgcttttctgacttggcagcgttgggcgttttaaatagacccattta

SEQ ID NO: 6 - DNA, 122 bp, synthetic construct
  source 1..122: mol_type = other DNA
caggaacttcactgtaatatatggtaagagacaagtagctctttatggccagtgattattcggtttgaatgaaacctgtgatttatgtccatttaactgaaaagccttgtaaattcccagtg

SEQ ID NO: 7 - DNA, 122 bp, synthetic construct
  source 1..122: mol_type = other DNA
ctgtcctcaaaagttgtatagggcatgatggcttgttttgtttggcaaaattctctagaacgtgattcattgtctgcaggtgattcatggttaggaaattaattctgaactctgccagtgat

SEQ ID NO: 8 - DNA, 122 bp, synthetic construct
  source 1..122: mol_type = other DNA
ttggagatgaggtcgtcgtggttggccaggtgagcgcggcagtgggcacagctatacctccggtgacaatcatccaagtaggcctgaaacgtcttgggctttgaaatccgcaccatggcggg

SEQ ID NO: 9 - DNA, 122 bp, synthetic construct
  source 1..122: mol_type = other DNA
atctcttccccatggagaaattcactccctgcctggcttgcaagatgttcacttgattgacgtttgctctgatgtgcaaactgctttgaacttcagacatttaattcccatcaggctgcagt

SEQ ID NO: 10 - DNA, 122 bp, synthetic construct
  source 1..122: mol_type = other DNA
acatatcacacaacaacctcccactcttacgagcaacaatccaaaacagttcctccaaagcgggcccaagggcagaggttctgtatatcattcaaagaccatctgtcgtgcaattcctgtag

SEQ ID NO: 11 - DNA, 122 bp, synthetic construct
  source 1..122: mol_type = other DNA
cagtctttgttaaatcacaaggcgcgcccttcactagccgacattttcatatttgttagacgcactgacctgaagttcacctcggccttggactttgcgcttctaaaaggtctatacagtgt

SEQ ID NO: 12 - DNA, 122 bp, synthetic construct
  source 1..122: mol_type = other DNA
gctaacacatccctaggccctgaccacccctgggactccttttgacagactgaaattggccgggagtgtggccagcctgtcagggtcacaggtcaccattaatctgccagaacaagggaggg

SEQ ID NO: 13 - DNA, 122 bp, synthetic construct
  source 1..122: mol_type = other DNA
caggatgactgttccccttccctcctaggtgctgatggtgcgctttgcttcgttgttcaacgtgaaggaccagacagtgatgttcctaagccgcaccacctacagcctgcaggagcttggtg

SEQ ID NO: 14 - DNA, 122 bp, synthetic construct
  source 1..122: mol_type = other DNA
agcagcttgtgcagagctagaactgtaacaattttatattccaaaggttttgtgtgtattcggttagttacagtaattatacccaataactgacagccaaaatcacgtaggagaaagcaaag

SEQ ID NO: 15 - DNA, 122 bp, synthetic construct
  source 1..122: mol_type = other DNA
cgcggcggccggccgggaggggggagcgggcgggggggccgtttagccgtgatagatccgcgcgccgcttgtctcttaatctccagagcgaccgatcgattcgctatttccctttgttgcca

SEQ ID NO: 16 - DNA, 122 bp, synthetic construct
  source 1..122: mol_type = other DNA
gccagagattcaactgcagtgcctgacacggaaccccaggaagtcacttaaatcaaagagcggaaactgcttggttcaaatttagaaagtgcacattgttttctattagaagtgccatttcc

SEQ ID NO: 17 - DNA, 122 bp, synthetic construct
  source 1..122: mol_type = other DNA
gactgctttttcgtgtgttgtaaaaggagtgagacctcagggaggcttggagcctcctcccgtgtaatgtccaagtcagatttgaaggactggtggcaggaaagctttggccttggggtgtg

SEQ ID NO: 18 - DNA, 122 bp, synthetic construct
  source 1..122: mol_type = other DNA
tagtacttttgggcacaatatgagaaaaggtggtgatttttaccatcataatctctgcatcgcctctttggtggtaggtttcgaccaaaagaattggaagagctatgattgttatagtaatt

SEQ ID NO: 19 - DNA, 122 bp, synthetic construct
  source 1..122: mol_type = other DNA
ttttgtctgcctgccacattaggggccatacggggatggcaggctctgagagaggggagtcgtgtaggcagcataattcactgtgaattgagaaaagaagtctaattctgataaatggtcga

SEQ ID NO: 20 - DNA, 122 bp, synthetic construct
  source 1..122: mol_type = other DNA
ggaggataaaagatttcacagagtttttgcatatcacagttttctcattatgagacccgacgaagattgctcagtgcatacatgatgtgttaaaagagtagggtgaaaagcagacacttgtt

SEQ ID NO: 21 - DNA, 122 bp, synthetic construct
  source 1..122: mol_type = other DNA
gagggtgtccgccggccccagcacccaggggacgcgctggaaagcaaacaggaagattcccggagggaaactgtgaatgcttctgatttagcaatgctgtgaataaaaagaaagattttata

SEQ ID NO: 22 - DNA, 122 bp, synthetic construct
  source 1..122: mol_type = other DNA
aagttgacgaaaggggagagcaactttttcctccccgctcgctctctccctctctctctccgagtggctctcagtcctggtcaaatcatattccgggcttttgaaatagtgggcgctagagc

SEQ ID NO: 23 - DNA, 122 bp, synthetic construct
  source 1..122: mol_type = other DNA
cttttaacattaaggatttgggctgaaaggccatggtgccagttgcgcaatttggcaaaacgtgggctgttacgttccgtttcaaacctttccctgtggcacagagagagagagagagagag

SEQ ID NO: 24 - DNA, 122 bp, synthetic construct
  source 1..122: mol_type = other DNA
aatactaatcaggaaagtcagaccctggaggtcagggagtaagtcaatcattctagcgatcgtctcagtgtggaggatcaaattagcctgaaaaattcagctgctgggaggagagggcgagc

SEQ ID NO: 25 - DNA, 122 bp, synthetic construct
  source 1..122: mol_type = other DNA
ggaaagtactgatggggagcggctctacaagtcgcctcagtgctcgaaccccggcctgtgcgtccagccacatcacattggagtcacaatcaaagaactggatctttatctggcttactttg

SEQ ID NO: 26 - DNA, 122 bp, synthetic construct
  source 1..122: mol_type = other DNA
aagttgtgttttacaatgcagagagtggaggatgctttttatacattggtgagggagatccgacaatacagattgaaaaaaatcagcaaagaagaaaagactcctggctgtgtgaaaattaa

SEQ ID NO: 27 - DNA, 122 bp, synthetic construct
  source 1..122: mol_type = other DNA
gcggtggccacggagtcggagggcagaggctcgtgaagagccggacggggaggagcgcagcggaagcctaacctcgtactccttccgctgctccagggccttatgaatccatttcagcctct

SEQ ID NO: 28 - DNA, 122 bp, synthetic construct
  source 1..122: mol_type = other DNA
ggccctcacccacgcggaagccggagcccgtgagcgtgtctgtgctgtggccgttctctccgatgagcgtcatgttggagccctgctgacaactgtcccgacactggcccttgagacaggtc

SEQ ID NO: 29 - DNA, 122 bp, synthetic construct
  source 1..122: mol_type = other DNA
gaccctgttgatgttgtaccgcaggatggacggaatgatttctactgctgggtttgtcaccgggaaggccaagtcctttgctgtgagctctgtccccgggtttatcacgctaagtgtctgag

SEQ ID NO: 30 - DNA, 122 bp, synthetic construct
  source 1..122: mol_type = other DNA
acttgagagaaggcttatttataaatgttcagggctcgggattcttgcggagctgatgtccggaggggaaggtcaccaaaatgcttgtttatagaagcaacagcaactgggaacaccataaa

SEQ ID NO: 31 - DNA, 122 bp, synthetic construct
  source 1..122: mol_type = other DNA
tcagggctaaatgatgggcctgtacattcctaaacagcccccagtggaaattgctgtatccggaagctcttttgtgcaagtaactagctgtattaaatgcaataattacagaaaagacactg

SEQ ID NO: 32 - DNA, 122 bp, synthetic construct
  source 1..122: mol_type = other DNA
ctccacgctcaaaatggggcctacatctgacaggcaggaatttagttaaaagcttttaaacgggaaacgtcattttgatgtcacgtttccatagcaacagagcaactgcataagctacaaca

SEQ ID NO: 33 - DNA, 122 bp, synthetic construct
  source 1..122: mol_type = other DNA
ccacgaggcccgagccggcgatgtcgtcgagcaggatggtgagcgtggtgttgaccagcacgttgcccagcagcagtgagcacagcaggtagttgccctgcctgcgcaccggctcgatgcgc

SEQ ID NO: 34 - DNA, 122 bp, synthetic construct
  source 1..122: mol_type = other DNA
ttggccaaatgggtcaaggggaactagggggtctgctaggccctttgtctgtcccaggtgcgtctcctgggtgtggtatctcagggccagccaactctggtcatcatggagttaatgacccg

SEQ ID NO: 35 - DNA, 122 bp, synthetic construct
  source 1..122: mol_type = other DNA
agtccatgaccttcccattcaatcttgaataatagcactgaattgagctgtaaaagcttccgaagtttgaagccttttgacaagttaatttagcagtatggtaaaaagtttttccccaagat

SEQ ID NO: 36 - DNA, 122 bp, synthetic construct
  source 1..122: mol_type = other DNA
cccgccgcccctgctgttcctgggccccctggcccccgctcaccacagcgggagccacagcgagtatcccatgagcagttccgggctgccctgcagctggtggtggacccaggcgacccccg

SEQ ID NO: 37 - DNA, 122 bp, synthetic construct
  source 1..122: mol_type = other DNA
gacctggagggaaactaggagatgcaaagggggctgtggccagagcaggggatgctagagcgacagaatgctgaagatagttgtcccagctacgagataaaagctacattacatgaaattaa

SEQ ID NO: 38 - DNA, 122 bp, synthetic construct
  source 1..122: mol_type = other DNA
gcattttcaccaaaaagtaattattgtaagtgacatttttactgctggcatgccataaggcgaactgtgcattcagaaggacaagttgctgaaacaaaatgacagggtactccgtgacattc

SEQ ID NO: 39 - DNA, 122 bp, synthetic construct
  source 1..122: mol_type = other DNA
cgggaggcggcttgctttcccgatttgcaggcgtgctttcttttggcagggagctgctgccgtctgtgctctggaccgtaaaacaagttttcatcaataaattaagataagcggaaagccag

SEQ ID NO: 40 - DNA, 122 bp, synthetic construct
  source 1..122: mol_type = other DNA
atgcaattcctggccataaattctctcccaagtacagatttagggagatctagaggactgcggcgggttgataggtaagttttaatctgacaaggccaaaaagtctattagatttatgggga

SEQ ID NO: 41 - DNA, 122 bp, synthetic construct
  source 1..122: mol_type = other DNA
aagtgtactgctgacagatgccagcatgcataaaggacacttcctataaaggcaaagctccggctaacagatggactaaagatgtgagatgcagaaagatttaccaatctaaataattcaag

SEQ ID NO: 42 - DNA, 122 bp, synthetic construct
  source 1..122: mol_type = other DNA
ggccaaaacccgaaatacagatgtggcagcggcagaagctctgtttagcaggccattatccgggatggctaagctccgctagctaaaagttacttcccattacttttgctttccagttttag

SEQ ID NO: 43 - DNA, 122 bp, synthetic construct
  source 1..122: mol_type = other DNA
acacatgcacgcacacacaaggcagcgatcgcaaaatcaactcacaggatctccaaatcgcgcagcgctcggaaggctccatcttcaatgcagctgatgtggttgttgtccagttgcctgtg

SEQ ID NO: 44 - DNA, 122 bp, synthetic construct
  source 1..122: mol_type = other DNA
ctgtgctcacatttctgaacctcttgcttttgttctctcttctagatgatatcagtgggacgctgcctacatctgtccttgtggctccgatggggtcttccttgcagtctttccccctacct

SEQ ID NO: 45 - DNA, 122 bp, synthetic construct
  source 1..122: mol_type = other DNA
tgtctttatagctgatgaaaaaaattgcagattattagcataaatgtttactcttcattacgctgatgacattgtgcactcgagatcttggtaatctttgggaaaattatgagtaattttta

SEQ ID NO: 46 - DNA, 122 bp, synthetic construct
  source 1..122: mol_type = other DNA
tacaagccagtaggtatacataatttcaacagagcactttgttgatttttccagaaacctcgaccttgaagatggtgagtagccagccaaagtacgatctaatacgggaggtaggccgaggt

SEQ ID NO: 47 - DNA, 122 bp, synthetic construct
  source 1..122: mol_type = other DNA
aaaccgctgacttttctattgcagacaacataggcggcccggaaggcaaaagggcccggacggcctacacgcgctaccagaccctggagctggagaaggagttccacttcaaccgttacctg

SEQ ID NO: 48 - DNA, 122 bp, synthetic construct
  source 1..122: mol_type = other DNA
tgtagccgtagccgtacctgccggagtgcatgctcgccgagtccctgaattgctcgctcacggaactatgatctccataattatgcaactggtagtccgggccatttggatagcgaccgcaa

SEQ ID NO: 49 - DNA, 122 bp, synthetic construct
  source 1..122: mol_type = other DNA
gaggccatagccacaatgcttaactgcatgcatctaacatggggctggttttaaccccagcgctctgacctaaactgctcagctcccggaaattccaaggtgagtggtggtgggggtggggt

SEQ ID NO: 50 - DNA, 122 bp, synthetic construct
  source 1..122: mol_type = other DNA
ctccattaagtccttttatttaaggaggatgtcattatcgcgcctccctgaggactattacggttctattaggcaaggccgcagacttcctaggtactgtctggggtctgattcctgggcag

SEQ ID NO: 51 - DNA, 122 bp, synthetic construct
  source 1..122: mol_type = other DNA
tattaacctctaacaccagtataaaaataggcagttattaaggggtaaatattactttgacgattattgccccagtctgctttctctgggagaggtaataaataaggcctctcatggggtcc

SEQ ID NO: 52 - DNA, 122 bp, synthetic construct
  source 1..122: mol_type = other DNA
tttggggcttgttgctgcagtccgtgctccagtaccgagtacctggctgggccctgggcacgcacaggggccgtagccccactgtgtgtgggagccatgaggatcctggctaacaagacaag

SEQ ID NO: 53 - DNA, 122 bp, synthetic construct
  source 1..122: mol_type = other DNA
ataccttgtgatgaaagtcaaggggatgagaaacagtgctgcagtaatgaacagatttttcggcaatgtttatgatctcattgcaaaccgcttcctgtgaggtggccccgagatatccagaa

SEQ ID NO: 54 - DNA, 122 bp, synthetic construct
  source 1..122: mol_type = other DNA
aagagaaaggtagagccctgcggaggggtcagggtaagacagaggccaggatgctactcacgtcggttttcatacatgctcctggactgggcccagattttaaaaggatctggttttttctg

SEQ ID NO: 55 - DNA, 122 bp, synthetic construct
  source 1..122: mol_type = other DNA
agtctagggccaaagcttttgggtgcagagcctccaaggttccaagataaacaaatctgtcggtgagagtgaccagagtgttaggtcagtccaagggtcacttataggtcatcctgctggga

SEQ ID NO: 56 - DNA, 122 bp, synthetic construct
  source 1..122: mol_type = other DNA
gctactgatgtgcactgtgaaaactcaataaaggggaacatctaatgaaaacagatacaacgtacaactgcaatcataggtcatgcatgaattattaacctcggggagttttctatcagttc

SEQ ID NO: 57 - DNA, 122 bp, synthetic construct
  source 1..122: mol_type = other DNA
tcagttactttttagtgaagtgttaaaaaatatgaagcttttctcctctctaaaattgcacgctgtctgtctgttattagggagataacagctcttataaatgtttcagtaagcgctaagta

SEQ ID NO: 58 - DNA, 122 bp, synthetic construct
  source 1..122: mol_type = other DNA
gagagggagagggagagtggggggataatttgggaagagaaaattgcagctgtgacaagacgtttatcattcggaagctttaattctattgaaatggcagctgctcagctccggggcacaga

SEQ ID NO: 59 - DNA, 122 bp, synthetic construct
  source 1..122: mol_type = other DNA
atttagatgccgatccagaagataaactattgacaagccagatgtatttttacattagagcgcctttacaatagtttatgttgagagtggaatataaattgcattattaaatatgaaccgct

SEQ ID NO: 60 - DNA, 122 bp, synthetic construct
  source 1..122: mol_type = other DNA
ttataattttttattttatgaaacaggacagggagcttagtgacttattactattcatttcgtttcaatgcagtaaatattgattcagttgggggttttcaatttaaaccactcggcgatta

SEQ ID NO: 61 - DNA, 122 bp, synthetic construct
  source 1..122: mol_type = other DNA
aatatggtgcataatgtaactctggcttgagccatatgtgtttaattttagaaaaacatgcgtgatgacaggttatgaaaggatgaaactattttagtctttttaaggattagaagcatgat

SEQ ID NO: 62 - DNA, 122 bp, synthetic construct
  source 1..122: mol_type = other DNA
aaagcacaggaaaatgtgaggaaaaaagggaaaaactaaaagcagcttagggtgtgttaacgctgctattctgtcagcttaaagagtattttatgcttgatcaacagctcttttttattgtg

SEQ ID NO: 63 - DNA, 122 bp, synthetic construct
  source 1..122: mol_type = other DNA
gcctggccagggctgggggccggagccccggcgaggcggtgccaggccccccgcgggcagcggacaatgttggcagtgatggagaggaggcatttcaaagcgttttgttcccgctgaccccc

SEQ ID NO: 64 - DNA, 122 bp, synthetic construct
  source 1..122: mol_type = other DNA
cgtcatcctctggcgtgacctggatgccaacctccacgggattggatgcttttttcatctcgattggtgaaggggaaggtggcttatccacagctttttctaagcagaggctgccattgcat

SEQ ID NO: 65 - DNA, 122 bp, synthetic construct
  source 1..122: mol_type = other DNA
ctgccatggcatgccgttaaagcttcaatgactttactgacccagaagggagactcagaccgtggagaggcttttgaaaagaagtggtgatctttcacgatttatgagcttgtcttaaagaa

SEQ ID NO: 66 - DNA, 122 bp, synthetic construct
  source 1..122: mol_type = other DNA
caaatgaatagggttttgtctatgaattagatcgtaaaatcatccatagcacagacagatcggctcactggctataaaacgtcacgtggggccattaaagtaagttttatggttttggggag

SEQ ID NO: 67 - DNA, 122 bp, synthetic construct
  source 1..122: mol_type = other DNA
gctctgatcagggagactgcgagcacaattaccttcttttcaaatccttctgtgacactgcgggaggaaaaaggactttgaaacttgaaaggaaagagcttgctttcaacctcaaaagctag

SEQ ID NO: 68 - DNA, 122 bp, synthetic construct
  source 1..122: mol_type = other DNA
gttgaagttcagtctggcgcagtgcagcattccacaagacctgctgcctctttgggcaggcgattaatctcatgatcgatagaccatatgattaggcactaatagctgataggaggagagaa

SEQ ID NO: 69 - DNA, 122 bp, synthetic construct
  source 1..122: mol_type = other DNA
cttccaagggcttaccactggtggatcaacaggtgctggtggttgcacttgtaggtttaccgcaacagtctgtggaggaggaagagtctgaaatggcaactggaccaaagcttctgcagctg

SEQ ID NO: 70 - DNA, 122 bp, synthetic construct
  source 1..122: mol_type = other DNA
ggttggagagaaagagctaccggcctgggacaaagtttagctataaagatcttccccagtcgggctctttcatgccccatcagaattgtaaatgaggggtctgtgggcagagctgccctttg

SEQ ID NO: 71 - DNA, 122 bp, synthetic construct
  source 1..122: mol_type = other DNA
cccactgtactctctctgagctggtcagacaacatctggagtactgtgtccggggctgggcgtcacattttcagagggacattgacaaacgagtgtgtccaggatggtgaggggtctgaaaa

SEQ ID NO: 72 - DNA, 122 bp, synthetic construct
  source 1..122: mol_type = other DNA
gcctcagccacccctaatcaggccgggggcgcagccgaaatccaggagctggcctccaaacgagtggacatccagaaaaagaggttttacctagacgtgaagcaaagctcccggggccgctt

SEQ ID NO: 73 - DNA, 122 bp, synthetic construct
  source 1..122: mol_type = other DNA
gaatactggtggtctgaaacgcaattagccctctgtgaaaggggcccctcacagccccctcggccaatcccctccgaggcactgctcacgttacaatggccttgaccacaatccattgagta

SEQ ID NO: 74 - DNA, 122 bp, synthetic construct
  source 1..122: mol_type = other DNA
tgtgacactgcggtgccttgcagattaggtgagctttcaatctctctttaaaaggggcggcgaagaggacccgcaggctgcaaggagcaacagcgatgggtaggcacattcaaaaccagaaa

SEQ ID NO: 75 - DNA, 122 bp, synthetic construct
  source 1..122: mol_type = other DNA
ggggaaggggaggagggggagaaaaaccctcactcaactgctttttccattcaataaattcgcttcagaatatattggattggggtttgatggctatttcgtgttttgcagagtgctgacac

SEQ ID NO: 76 - DNA, 122 bp, synthetic construct
  source 1..122: mol_type = other DNA
cctcctctggccctggccctttccagaggacaatagccttcatgcaagggaaaattccatcgcccagggttcaagtctccaaggagttgctggggcctctatttagcctggagtgggctctg

SEQ ID NO: 77 - DNA, 122 bp, synthetic construct
  source 1..122: mol_type = other DNA
cacagaacagcttcccgcccgccagcccgcctcaacccacctcctctggagctggctttccgcttatcttaatttattgatgaaaacttgttttacggtccagagcacagacggcagcagct

SEQ ID NO: 78 - DNA, 122 bp, synthetic construct
  source 1..122: mol_type = other DNA
cttgtaggtcactaaattctgtacaaaagttcattgctgctgtcgctgttattacttcagcggttgttaaaactgtggttactattaacctggttacagaaaattttccacttgacacctcc

SEQ ID NO: 79 - DNA, 122 bp, synthetic construct
  source 1..122: mol_type = other DNA
attcaggcctcagaaaaatttattcaagtcaactggtttcggtccgcaacaaaaaagttacgagaaaaaagtgagtgtgttcttgccctaacattctggatctgcttgcaagcaaaatgata

SEQ ID NO: 80 - DNA, 122 bp, synthetic construct
  source 1..122: mol_type = other DNA
ccacttccctagcccctgctcaccagcccacccagccttgcctgacaggcccggcactcacgtcttgatgacctgtcccaaatcatatctgtcagtcacctccgatggctggttatagttct

SEQ ID NO: 81 - DNA, 122 bp, synthetic construct
  source 1..122: mol_type = other DNA
aggacaaaaaggattgaaagttgtatttaatcagggtcagagaggtggtgttcagagtgacgggcccacaggcaggctggccttttatgcttgccgacgcagatggtcccagagcattcagc

SEQ ID NO: 82 - DNA, 122 bp, synthetic construct
  source 1..122: mol_type = other DNA
cagggctctgcgttcaagtcctctgccaggagagagttaagggcttgggtttaatttgcccgttaatcacagttaaaagaagtaatgagcgtctccccaaagcctgggtgtttctctgcaga

SEQ ID NO: 83 - DNA, 122 bp, synthetic construct
  source 1..122: mol_type = other DNA
cttgagggcggccacatactctcaactccccgccccatgaccaacgggaccctgggaggacgcaagggagcttctaccccattgcagttgcacaaaagaaattgaacacttttgagttgccc

SEQ ID NO: 84 - DNA, 122 bp, synthetic construct
  source 1..122: mol_type = other DNA
cactagggttaacagaaaatgacaaaccaggaatggatgagggaggcgccagcacagcagcgggatgtcttttgatttgcttctgttatttgcatgtctctcttgtgatgtctgtgaaatgt

SEQ ID NO: 85 - DNA, 122 bp, synthetic construct
  source 1..122: mol_type = other DNA
catgtatgggctgggtatggtgggggtggaggtgggtttgctccgagctccgtcgatgagcgggtgtagttcccagttctggctcttttcaaggaggaggagcagaaagaggatgaggagga

SEQ ID NO: 86 - DNA, 122 bp, synthetic construct
  source 1..122: mol_type = other DNA
gctctctggagagacaagcccagctattgtttggctgctctcatgttagcgagtgccctgcggtaaccctcgtgctctgaaatactcatattggttatttcaaagttgggtgcagaattgtg

SEQ ID NO: 87 - DNA, 122 bp, synthetic construct
  source 1..122: mol_type = other DNA
catgggttagttgaagtgatattttccgctaatggtagtcgtacatcattgctgtcagctcgggttaggaggcaagcacattacagctccattttacctctgcaggcatctgggattatttg

SEQ ID NO: 88 - DNA, 122 bp, synthetic construct
  source 1..122: mol_type = other DNA
ttccatcggcatctgctaacacactaactcataaacaaggcccggctggatcaggtggcacggaataatacaggctaatgaaatacagcacagctttccattactgttagtttttacagtgt

SEQ ID NO: 89 - DNA, 122 bp, synthetic construct
  source 1..122: mol_type = other DNA
gaccccaatttccgaggatggcctcaccggggcttttttccctcatttcgaagcctgattcgtgatgagagactaattcgttacgctgaaacattaacggcaatttaaggggtctggcaggg

SEQ ID NO: 90 - DNA, 122 bp, synthetic construct
  source 1..122: mol_type = other DNA
tggaaagaaaggcatcattaaaaaaataatcatatatggcaaacccgctgaaggggtctacgtgagaataatgaatgtggaaatacaactaaaaacatggcactcaaaacttaatcggaaaa

SEQ ID NO: 91 - DNA, 122 bp, synthetic construct
  source 1..122: mol_type = other DNA
gctggttaacaggctccgagctgctcagcgacttgttttaagcattttctctctcgctctcgcttttaatcttgtctctagtggcgtgtgtaggggggaggactttatttccattggctaag

SEQ ID NO: 92 - DNA, 122 bp, synthetic construct
  source 1..122: mol_type = other DNA
tagagtgagggaagagaaaacagcggctgtaaccttgtctgactgtagctgattctgaaacgcatgaagttgtcccacaccaaggtcagcgactggcaggttctctacaaggtaaggccttc

SEQ ID NO: 93 - DNA, 122 bp, synthetic construct
  source 1..122: mol_type = other DNA
tgctgccttggcagaacatgactgatgtactcgcgttaattagggctccagtcagcagcccgtgccagctggaaactgagaaacaagcgtaaattattctgtcatcaatcagctcgaaaatg

SEQ ID NO: 94 - DNA, 122 bp, synthetic construct
  source 1..122: mol_type = other DNA
agatggcccctcactgttgatggttcgattaccatgatcacccacagcttcagaggagtgcgaagaaaagaaattgctttctttgtcaaggtgttttcaattcaattctcacccattagccc

SEQ ID NO: 95 - DNA, 122 bp, synthetic construct
  source 1..122: mol_type = other DNA
actaccagttgcataattatggagatcatagttccgtgagcgagcaattcagggactcggcgagcatgcactccggcaggtacggctacggctacaatggcatggatctcagcgtcggccgc

SEQ ID NO: 96 - DNA, 122 bp, synthetic construct
  source 1..122: mol_type = other DNA
gcgccgccgcgaccccagcacagcgcctaagccccagctttgatgtccaagaacagaaaacggcgattaaatgttgttttaaagagggagtgctcactgccctcccatctgtcaagagaaag

SEQ ID NO: 97 - DNA, 122 bp, synthetic construct
  source 1..122: mol_type = other DNA
ggccttccatcatgggagatctataacatttacaatcccaaaacaaaagaccccgagagccgaggacagaattcatcttcccagctgacaagccccgttaattaacgacttagaaatcgatg

SEQ ID NO: 98 - DNA, 122 bp, synthetic construct
  source 1..122: mol_type = other DNA
atacaaggaggcaactgataagaagcgcatcagaagggcagttaattgcctccagtctctcggtgctgggaatatttaaaatctatgggattaaaggcagattaaaaactgtggaagcaggg

SEQ ID NO: 99 - DNA, 122 bp, synthetic construct
  source 1..122: mol_type = other DNA
cacgtgctgctgaggcaggactccggcaggttttttagagttttattttatgggaacaggcggttacggctccccaagtgctgtaaaagttaatgtgatggcctccctgtgcctgtgctcat

SEQ ID NO: 100 - DNA, 122 bp, synthetic construct
  source 1..122: mol_type = other DNA
cttaagggtgctggagttgtgtgttccagggcttgcccctactgataacacccttgttggcgtgaaagtgcaatgcaaattatgacagttacaaccttctgttttgtttaacctgtcagtag

SEQ ID NO: 101 - DNA, 122 bp, synthetic construct
  source 1..122: mol_type = other DNA
ggaaaagtaaaacactacacagcaacatactttatacttggtctaaattgaggtctagcacgcccagaaatttccctgagctttatcatgattcctggaggcagtctgatccgaggcagatc

SEQ ID NO: 102 - DNA, 122 bp, synthetic construct
  source 1..122: mol_type = other DNA
ctgagactttaaaggaaaatggattttttttttgagcgtactttaatgaagaagataaagcggcccggctggtgtcccacaggctcggcgcagtaagttcagattattgctcagtctgtcag

SEQ ID NO: 103 - DNA, 122 bp, synthetic construct
  source 1..122: mol_type = other DNA
gagcagccgaagggatcggtaagcataatttcagaggccagtaatattttatataatcgccgagtggtttaaattgaaaacccccaactgaatcaatatttactgcattgaaacgaaatgaa

SEQ ID NO: 104 - DNA, 122 bp, synthetic construct
  source 1..122: mol_type = other DNA
ggactgacattttgtactgctgtgagatgggaaaacgtgtgacatttttattcatggtggcgtacgcccttgtaatgatcagctctggtcgatgtgcttaccgcagcagcccctgagccctc

SEQ ID NO: 105 - DNA, 122 bp, synthetic construct
  source 1..122: mol_type = other DNA
gaccatttacgacttgctatggtgtctctattgtttaccagcgacattcgcagaaaaaggcgacagctgggacagaaagctgctactcaacgaacaatataatcccgtgaaagtaatcaaca

SEQ ID NO: 106 - DNA, 122 bp, synthetic construct
  source 1..122: mol_type = other DNA
gtgagagggaacgcaacaacacaaaatttttcatttttctccataaatcatttcagcgtgcgataatatttgttttttctaatgagtctcttgtgatccattccaggagctaacatcagggc

SEQ ID NO: 107 - DNA, 122 bp, synthetic construct
  source 1..122: mol_type = other DNA
gttttaactgtttaaaattaatgagaagggcagaatggttgtcaaagcaatattgaaagccgggcagccaggaccgaataagcacaaaaatccctctagagtgttaaataaacagagctcca

SEQ ID NO: 108 - DNA, 122 bp, synthetic construct
  source 1..122: mol_type = other DNA
gcgtccggatcagccacaaacagtaacaaccatctaatcgagcctcagatccgccggcctcgtagcctcagctcgccaacagtaactttatctgccccgctggaggtgagacggctacctca

SEQ ID NO: 109 - DNA, 122 bp, synthetic construct
  source 1..122: mol_type = other DNA
accatcgaaaagtacctgaaatggttgctgctgggatgaaaaagcagcagaaacctttggcggaagctgggttgctatagcaacgggagtcccagtctgcccatcctttcctgtcacaacct

SEQ ID NO: 110 - DNA, 122 bp, synthetic construct
  source 1..122: mol_type = other DNA
gccccatccccagaagttccagcactcgacacgcatactcattcaccagcttgctcctctcgtcgtgcatgtcctgggactttttgacaggaggtgtgagcttagcagctggggctgggcag

SEQ ID NO: 111 - DNA, 122 bp, synthetic construct
  source 1..122: mol_type = other DNA
cctctgtgttgcttttgtcacgttaattcccattttgccaacaccaggggcccaggcagacgagttaccaaggaacacaaaattttaattagtgtattttaatcattcaggctttccgagct

SEQ ID NO: 112 - DNA, 122 bp, synthetic construct
  source 1..122: mol_type = other DNA
ccacagcggaattcattaggctcaacactgcagggcccgctctctgtgcgttaatgacatcggagggtagggaaacgagacagccaccacgcgagatgaaataaacatgaggactgtcagca

SEQ ID NO: 113 - DNA, 122 bp, synthetic construct
  source 1..122: mol_type = other DNA
gttggcactgctcaacttgttctcatcctaagcagggttgatagaagaacatcatgaggacgaagtggtaacatttcaagttgtcaaagggtaaagggaacaggaataagaaaatacaaaac

SEQ ID NO: 114 - DNA, 122 bp, synthetic construct
  source 1..122: mol_type = other DNA
tggcgagacgagcgggagaatggcgcagcccgcgcgcgcctcagacccaagggcaagagacgaataaatgttgattagtgttgattttgtccgacaaacgcctgtcgagtgacagttgggct

SEQ ID NO: 115 - DNA, 122 bp, synthetic construct
  source 1..122: mol_type = other DNA
tttctttttcaagctacagcttgacattttgcaaactctctagagaccaaacagtgaaatcgagttactgcaggagtgaactgtaaattccatgaaaccatgtaatatatcttaacacagat

SEQ ID NO: 116 - DNA, 122 bp, synthetic construct
  source 1..122: mol_type = other DNA
ggccggagcccgagcggccgacgctgagatccatgccattgtagccgtagccgtacctgccggagtgcatgctcgccgagtccctgaattgctcgctcacggaactatgatctccataatta

SEQ ID NO: 117 - DNA, 122 bp, synthetic construct
  source 1..122: mol_type = other DNA
taaaaactggctgaatatttagatggaagagccatttggaagcagtttatttagacgaaccggcaatgacccacaacagggagacatcaagcgtgaaatttactagaaattagcaccagggt

SEQ ID NO: 118 - DNA, 122 bp, synthetic construct
  source 1..122: mol_type = other DNA
gccccgagaggtctcgcccgcgctgagccctgtccctggggcccccgcccctgttgcgcccggagctcagccacagatgtccagccacaattctcggttggccgcagactcgtacaagaatt

SEQ ID NO: 119 - DNA, 122 bp, synthetic construct
  source 1..122: mol_type = other DNA
cgcacacacaaggcagcgatcgcaaaatcaactcacaggatctccaaatcgcgcagcgctcggaaggctccatcttcaatgcagctgatgtggttgttgtccagttgcctgtggaaaagcag

SEQ ID NO: 120 - DNA, 122 bp, synthetic construct
  source 1..122: mol_type = other DNA
cgctggcagcgtagctgcgggcgcgctctccggagccaaagtggccggagcccgagcggccgacgctgagatccatgccattgtagccgtagccgtacctgccggagtgcatgctcgccgag

SEQ ID NO: 121 - DNA, 122 bp, synthetic construct
  source 1..122: mol_type = other DNA
aactgtaggcagcttcctttgggaccaacatttcctggctaattaaaatgaatgttgtatcgatgaaagattattttaaaatggcactgatagtgtttagacattgtcataacatcagccgg

SEQ ID NO: 122 - DNA, 122 bp, synthetic construct
  source 1..122: mol_type = other DNA
gaaggaaaacaaaatatctacagttgtcgagacaagaggagaaaaatagaggcaaacatccgtaattttctctgtggggctttgcggtaattgagccatttggataaaaataaaggcagcag

SEQ ID NO: 123 - DNA, 122 bp, synthetic construct
  source 1..122: mol_type = other DNA
tcgctcacggaactatgatctccataattatgcaactggtagtccgggccatttggatagcgaccgcaaaatgagtttacaaaataagagctcatttgttttttgatatgtgtgcttgattt

SEQ ID NO: 124 - DNA, 122 bp, synthetic construct
  source 1..122: mol_type = other DNA
aatgctgtcccatttggaaagcacttctttaaggcagcaccgattaaaattcatgaaaggcggcttgcttgcataaataaattaaaagatgcaaaactaaagtttaaacacccatacacaac

SEQ ID NO: 125 - DNA, 122 bp, synthetic construct
  source 1..122: mol_type = other DNA
gtgtcataaggagttaataggacacagctgtactggctctttctaataagcagcttctgtcgctttggtcaccacaggtgtctctgtaacctgcaaactcttattactagtgtatcaaagtt

SEQ ID NO: 126 - DNA, 122 bp, synthetic construct
  source 1..122: mol_type = other DNA
tattccaatctctagggagccctttcccctcttgaaaatattttggggtttcacaaaaatcgtttgcctgtctcagtggattgaacctcaaagactcctttgacgcaagtgttattttgacc

SEQ ID NO: 127 - DNA, 122 bp, synthetic construct
  source 1..122: mol_type = other DNA
gatcgtctcagtgtggaggatcaaattagcctgaaaaattcagctgctgggaggagagggcgagcagctgtcaggggcagcatcgagatgcactaatgaaccagatgaatagtgcaaaagct

SEQ ID NO: 128 - DNA, 122 bp, synthetic construct
  source 1..122: mol_type = other DNA
cccacctcccctttcctcacttagtgctctccttcccccatcctgctcccaggcatcatgcgaaggaactctctgagtggcagcagcaccggctcccaggagcagcgggcaagcaagggggt

SEQ ID NO: 129 - DNA, 122 bp, synthetic construct
  source 1..122: mol_type = other DNA
aaaaatcagggatggtacgactaatggcttacttatgtgcaattgttgttaaccgttcatcgtttactgctctccgaacttcagcacggtggcgttctgttgagatgctatgaagaaaaata

SEQ ID NO: 130 - DNA, 122 bp, synthetic construct
  source 1..122: mol_type = other DNA
aaccaagtgctctggaattcatgaagaaatcaggggtctttagcctgctgtttttcttggcgaggccttatcacaaattagtggggaagcccgaaggttagggtttcagttagttcagcaga

SEQ ID NO: 131 - DNA, 122 bp, synthetic construct
  source 1..122: mol_type = other DNA
gattgcggcagcaagtcagcagccaaaaccattaatctttgctgcattccccaactggcccgagtagtaaagaagataataccccagcagaatcagtgacccactacaaattggtcctgaaa

SEQ ID NO: 132 - DNA, 122 bp, synthetic construct
  source 1..122: mol_type = other DNA
acctcaggatggcctgattggctaccctgctctccagccaatccagtgcaagctctcatccgagattcactgaagtggtccatatccatctggcagcatcttctagttctgtcggggtgtca

SEQ ID NO: 133 - DNA, 122 bp, synthetic construct
  source 1..122: mol_type = other DNA
gggtatgaaagggagaaaagaggccttgaagcctttcatatcatttgagtttttcaagtgcgttatttttggggagggtggatgtatgaaaagccaattggagttttttttgttttaaagtg

SEQ ID NO: 134 - DNA, 122 bp, synthetic construct
  source 1..122: mol_type = other DNA
tgatccactggagcagagcctgggaagccttcgaggtgggcttgtccccagccagagaggcggaaaatcaatttggctggcaaacgagagcccccaagagcttagtccacgctccataatcc

SEQ ID NO: 135 - DNA, 122 bp, synthetic construct
  source 1..122: mol_type = other DNA
atggtaggtggcgtcgggcttgaacggatggttcttgccgtgggagacgatatccaccgccgccagagcttcggcgcgtgccagcaggctctcatcaaagcttccaaatatattaccctgca

SEQ ID NO: 136 - DNA, 122 bp, synthetic construct
  source 1..122: mol_type = other DNA
ggttgaacgctgaggcagtttcaccgcataatttttttgttgttgttgaatgtcacagggcgggttagtgtgaccttgagctgcatcagcagcacagcaagggtcgagatgcctgcaggcgc

SEQ ID NO: 137 - DNA, 122 bp, synthetic construct
  source 1..122: mol_type = other DNA
cccttgtccttcaggatgagaccctgcccttgattttactcccttcccctgttcatcttccgacagagtgtaaatgtaaagcagcgggcctgtgaaataattcaaaggcaatggaccagggg

SEQ ID NO: 138 - DNA, 122 bp, synthetic construct
  source 1..122: mol_type = other DNA
cctttttttatgcattttaaatcaacacacgcttgctctgaatttatgcatgtggatagacggagatgtaatttaaacacatctgttcaaatatgcatttcagatcaacttggtgccgattc

SEQ ID NO: 139 - DNA, 122 bp, synthetic construct
  source 1..122: mol_type = other DNA
tgtttttattacctgtttgactcaccgccttattgcttctccccttaatggggccgtaaccgtggaaacgaggaataaacggccaccagaaaatgagatgattaaacaggtcaccctggcaa

SEQ ID NO: 140 - DNA, 122 bp, synthetic construct
  source 1..122: mol_type = other DNA
aaaagtggcaagagtccctgttaacagccctgacctgagcggtaatgaagggagagtgcacggcaaagtgtccattatcagcaggtcgggagcagtaaattggagcagtttgcttcaatctt

SEQ ID NO: 141 - DNA, 122 bp, synthetic construct
  source 1..122: mol_type = other DNA
ttataataagcaaaaagattgtgtgtatgtatgtttaatataacatgacaggcactaggacgtctgcctttttaaggcagttccgttaagggtttttgtttttaaacttttttttgccatcc

SEQ ID NO: 142 - DNA, 122 bp, synthetic construct
  source 1..122: mol_type = other DNA
tggtgacagaaggtgctttagtgttaggatggggctgggaattgccgcggattagccatgcgctcctggggtaattaggaccatgtgttcttcctaaaacagggctggggccacagggctgc

SEQ ID NO: 143 - DNA, 122 bp, synthetic construct
  source 1..122: mol_type = other DNA
tatttgccaggagacagttgggtacataaacttttttatttcttgattcatgggtctattcggactgtagattgctctgccattctgagttcagcaatttgaataaattatgctgaaaatca

SEQ ID NO: 144 - DNA, 122 bp, synthetic construct
  source 1..122: mol_type = other DNA
tgtactgcctaggcaaatgaaggaagacatctgtagctcccgaatactggtggtctgaaacgcaattagccctctgtgaaaggggcccctcacagccccctcggccaatcccctccgaggca

SEQ ID NO: 145 - DNA, 122 bp, synthetic construct
  source 1..122: mol_type = other DNA
ctggcgagtgaaggatcactgtccgtcccaaggacacccgccataattaagcgaggctttcggccccagaaagtgcagattcaggttttaatacacaaaattatttcaggagcagtgaatcg

SEQ ID NO: 146 - DNA, 122 bp, synthetic construct
  source 1..122: mol_type = other DNA
gtgctgtctcattgctttgcaggctgcctgctgtttcccggggagatcatgaaacgaggtcgccttcccagcagcagtgaggattctgacgacaatggcagtaagtcctgctttgttgttct

SEQ ID NO: 147 - DNA, 122 bp, synthetic construct
  source 1..122: mol_type = other DNA
tggcagcgtagctgcgggcgcgctctccggagccaaagtggccggagcccgagcggccgacgctgagatccatgccattgtagccgtagccgtacctgccggagtgcatgctcgccgagtcc

SEQ ID NO: 148 - DNA, 122 bp, synthetic construct
  source 1..122: mol_type = other DNA
ggagagtggggggataatttgggaagagaaaattgcagctgtgacaagacgtttatcattcggaagctttaattctattgaaatggcagctgctcagctccggggcacagagcagccgggag

SEQ ID NO: 149 - DNA, 122 bp, synthetic construct
  source 1..122: mol_type = other DNA
aaaaaaaaaaaagcagccccggctgccaactcaccatccaccaagttctggccgagatgtcgtagcagagctgccatttgatggagccgtcggaagctttccctgccattacgctgtcagca

SEQ ID NO: 150 - DNA, 122 bp, synthetic construct
  source 1..122: mol_type = other DNA
cgtacctgcccttctggagccgcttatgacggggttctaatgaagcacgactctgggagccggagacccccgtggacacggcagttcaaagggaatatttcaacaatgattacattttgtaa

SEQ ID NO: 151 - DNA, 122 bp, synthetic construct
  source 1..122: mol_type = other DNA
atgatttaggattttcccaacattttatggcaatttgtttcatttgccgcaggcccgtaacgatgcaaattgccataaagtgggataatcaggcagtagaagtgtcataaaactgcaggaga

SEQ ID NO: 152 - DNA, 122 bp, synthetic construct
  source 1..122: mol_type = other DNA
tattctctaggacttcgactgccttaaaatatagacttgcatttatcttggccacattaacgagatctgtttgaccaatacatttatatttatgatgactgagggcagattatggttgttaa

SEQ ID NO: 153 - DNA, 122 bp, synthetic construct
  source 1..122: mol_type = other DNA
tgtcgatgatggcctgtgaacatccaagggggaaaatcatcttttatcactattgtgtaccgtgccggtccagatggaggataaaacatgcatgagttgcccgcagctctgaaaataattat

SEQ ID NO: 154 - DNA, 122 bp, synthetic construct
  source 1..122: mol_type = other DNA
tttttgagcgtactttaatgaagaagataaagcggcccggctggtgtcccacaggctcggcgcagtaagttcagattattgctcagtctgtcagaatgggctgcttccctcagcagcagcca

SEQ ID NO: 155 - DNA, 122 bp, synthetic construct
  source 1..122: mol_type = other DNA
cgtgccaaaacccgaattgtgggcagaactgataatggctggcaccgagtggccaaaacccgaaatacagatgtggcagcggcagaagctctgtttagcaggccattatccgggatggctaa

SEQ ID NO: 156 - DNA, 122 bp, synthetic construct
  source 1..122: mol_type = other DNA
ggggggcgggccggcgggcggagggctggagcgcgggctggcgagtgaaggatcactgtccgtcccaaggacacccgccataattaagcgaggctttcggccccagaaagtgcagattcagg

SEQ ID NO: 157 - DNA, 122 bp, synthetic construct
  source 1..122: mol_type = other DNA
atccttcttagttagccttggtagctcaacacctttggtatctcactcacttaactgccacgcctactcttgttggatccggttataaagtaaactatgttctttgagaactgttgcataca

SEQ ID NO: 158 - DNA, 122 bp, synthetic construct
  source 1..122: mol_type = other DNA
taagcagcaaactctctggtgacatagcaattatggctctgaaatagatttggtgcgtcacggaagataagggttttgaaagtttctattgtgattatagcctgtaactcccacaaataata

SEQ ID NO: 159 - DNA, 122 bp, synthetic construct
  source 1..122: mol_type = other DNA
tgtggatgagggagggtcacagcacatgctcagtcctgtcagagtgaggggaagtttctgcgttctgcgtgaaggcttaggtcatagcacaagctcagtgaaagctctatgaggtctctgag

SEQ ID NO: 160 - DNA, 122 bp, synthetic construct
  source 1..122: mol_type = other DNA
tctctactgcccgctgtgccagggcggctgccgagcactggtgcggaaccactatcggaacgagtttgcacttctgtttacaaagcaatcctgccatcaaaagaggaacaaaatcaccactt

SEQ ID NO: 161 - DNA, 122 bp, synthetic construct
  source 1..122: mol_type = other DNA
atggaatgcagaaagccttcctctcctaatggagtatataattcaggtgccctcaaggcacgctttcaggaattcttttgcctgaggtaatgggaagctaaggttgctttaaggccttgatt

SEQ ID NO: 162 - DNA, 122 bp, synthetic construct
  source 1..122: mol_type = other DNA
cccataatgccattgatcaatgatttcctttcagcagtgatgtaattaaagggctttagtcggcttctgctgtgttgccttgaaatttattattctggagatttaaagaagatcgatagggt

SEQ ID NO: 163 - DNA, 122 bp, synthetic construct
  source 1..122: mol_type = other DNA
aaaactaatgagaatccctcaatggatccttcacccaccaaacaagatttcttcagaaaccgacttgctcttgcaaatgaccttgaccaaggaacagctgtgtaaaacatacttaaagttgt

SEQ ID NO: 164 - DNA, 122 bp, synthetic construct
  source 1..122: mol_type = other DNA
gccgacgctgagatccatgccattgtagccgtagccgtacctgccggagtgcatgctcgccgagtccctgaattgctcgctcacggaactatgatctccataattatgcaactggtagtccg

SEQ ID NO: 165 - DNA, 122 bp, synthetic construct
  source 1..122: mol_type = other DNA
ccgcttcttctggcctgtcaaaaagcaagtgcaggtttggatggcagggaacaaagttgacgaggaggctgaacactgcatgaagagaggaccgagccaatgagagaacaacggcagcaaca

SEQ ID NO: 166 - DNA, 122 bp, synthetic construct
  source 1..122: mol_type = other DNA
acatgaaaataacccaacatactagacatgaagaacacatcttatagacttctcctcgtgcgatttgcaatctgtgatggattaccaggaagacagactgcagatttattggacttctgtag

SEQ ID NO: 167 - DNA, 122 bp, synthetic construct
  source 1..122: mol_type = other DNA
cattaaaatcctgctgaaggtcaccatggtaaccgcctgctgaggcttcactgctgcactcggcatctcctaatcagctaccatgtgaaatgcaaattcagacagtctgaaaattagtcaag

SEQ ID NO: 168 - DNA, 122 bp, synthetic construct
  source 1..122: mol_type = other DNA
cagctatactgggtactgacacaagtggggggaaatataagaaaaaagcactgctgtgatcgaatgaatgcgaccatatgagctatattggagccgtatgcatcctatttagctgatatcaa

SEQ ID NO: 169 - DNA, 122 bp, synthetic construct
  source 1..122: mol_type = other DNA
tcagaatgggctgcttccctcagcagcagccacagccagcaacccgctctggcccctcgccgcgccattcctgatgacaaactgccagccagctcccacaaaagcacgattaaaatgtcagc

SEQ ID NO: 170 - DNA, 122 bp, synthetic construct
  source 1..122: mol_type = other DNA
gttagctgtgcatggtggtaagtaagatccagctgcaggggattatggctttgagtgggtcggggagcttggaatgcattcagttccatcaggggatttgtgtccctcgtcatcagccttaa

SEQ ID NO: 171 - DNA, 122 bp, synthetic construct
  source 1..122: mol_type = other DNA
ctgggtttgtcaccgggaaggccaagtcctttgctgtgagctctgtccccgggtttatcacgctaagtgtctgagactgacatcggaaccagagggggactggttttgtcctgaatgtgagg

SEQ ID NO: 172 - DNA, 122 bp, synthetic construct
  source 1..122: mol_type = other DNA
agacattattttcctccaatagaaggcctattcttgaagaacaataaaagcacagagaagcgttagtctgcttacggatagaaataaatcaaaatgaataagagggttgatggaaatagtct

SEQ ID NO: 173 - DNA, 122 bp, synthetic construct
  source 1..122: mol_type = other DNA
cttttttccttttttactgccgtcatttcttgtgcttgttttttccccagtgagggttgtcgaagagaaaatacaatgaagaatgttggaagtcgcaaatatgcatttaattccctgcagct

SEQ ID NO: 174 - DNA, 122 bp, synthetic construct
  source 1..122: mol_type = other DNA
agcaggttgctggattctcatttatcagccttaagagggatggagtgataaatgcataaccgtccctttaatattttatgcaggtgctaacgcggcttggctgtcaccttcaatgcatcacc

SEQ ID NO: 175 - DNA, 122 bp, synthetic construct
  source 1..122: mol_type = other DNA
caaaagtcatcagccagtgaatgttcttttctgcaccacaattaagacgaatcaatacgacgtgccacaaatggtattttactctcaataagaactcgagctgagtttattcagaatatttt

SEQ ID NO: 176 - DNA, 122 bp, synthetic construct
  source 1..122: mol_type = other DNA
agccaaagtggccggagcccgagcggccgacgctgagatccatgccattgtagccgtagccgtacctgccggagtgcatgctcgccgagtccctgaattgctcgctcacggaactatgatct

SEQ ID NO: 177 - DNA, 122 bp, synthetic construct
  source 1..122: mol_type = other DNA
ttagttctgtccagctaattcttcccactactcctgcctgccgcttttactctggccatccgttcagtccagccgactcagaagtaattttatacgcagcagagagcgggtggaattgattt

SEQ ID NO: 178 - DNA, 122 bp, synthetic construct
  source 1..122: mol_type = other DNA
gagcgcgggctggcgagtgaaggatcactgtccgtcccaaggacacccgccataattaagcgaggctttcggccccagaaagtgcagattcaggttttaatacacaaaattatttcaggagc

SEQ ID NO: 179 - DNA, 122 bp, synthetic construct
  source 1..122: mol_type = other DNA
cagttccggctatatgacatttgggtgccaaatgaatagggttttgtctatgaattagatcgtaaaatcatccatagcacagacagatcggctcactggctataaaacgtcacgtggggcca

SEQ ID NO: 180 - DNA, 122 bp, synthetic construct
  source 1..122: mol_type = other DNA
gagttggggccgccagggaagtgtggggggcgggagacaatgtcggccccgtggtcggtgcgggccctggcatttgcccggaacgtcagcctgtaggtctcaatctgcagtgacatgggagg

SEQ ID NO: 181 - DNA, 122 bp, synthetic construct
  source 1..122: mol_type = other DNA
gatggcaagccatgtgtggcaggtgcgctgacagccattgtggccggcgaggaggagctccgtggcagctataactgggactacctgctgagctggtgccctcagttccaaccactggccag

SEQ ID NO: 182 - DNA, 122 bp, synthetic construct
  source 1..122: mol_type = other DNA
cttgggcccgccctcaccatcctgccaacatccccggcgaatgtcacccccttgcttgcccgctgctcctgggagccggtgctgctgccactcagagagttccttcgcatgatgcctgggag

SEQ ID NO: 183 - DNA, 122 bp, synthetic construct
  source 1..122: mol_type = other DNA
tagtggttataggaaatgtttatttcttttttgtttgtttatagtaaatctgatatgtctcgcttgcgattagctgctggtagtgccataatgaagcttgctcaggaaccttgttaccatga

SEQ ID NO: 184 - DNA, 122 bp, synthetic construct
  source 1..122: mol_type = other DNA
aaacattaaactgaattgtttatgttagtgctgtactcagcgtgtcatacatcaggccaccgtaatctctcatggaatgtaaaattctgtcatgaatcatggcttgtatatcggaaattact

SEQ ID NO: 185 - DNA, 122 bp, synthetic construct
  source 1..122: mol_type = other DNA
gacaccattcattaattattctgccgttgcagcttaacactgactggaaaggtctctcttcggacagatggcggggacgatgttatttatatcaatcagccaaaatcctcaacaaggattac

SEQ ID NO: 186 - DNA, 122 bp, synthetic construct
  source 1..122: mol_type = other DNA
gactccccgtctactccagcacattccatcgcctggagaccaaagccaccaaatgatttgcggtcagagtgatttggggctggttagagatgatttaaaaggaaggttatgggcatatgaag

SEQ ID NO: 187 - DNA, 122 bp, synthetic construct
  source 1..122: mol_type = other DNA
agggttactgtgctctgaccacttaggctgaaaatgaaagaggcattaattaagtaataacgatcaaggtatggggcatttcctggggattaatgaccagctgggggagttgatagcccaca

SEQ ID NO: 188 - DNA, 122 bp, synthetic construct
  source 1..122: mol_type = other DNA
agccccgccagcacagcagttggtggtgagcagtgaagtggccggtgtgggcggcttgggcgctgggcggctgtctcctgacaccagaaactctctttatgaaaggatcaagtcggtagcat

SEQ ID NO: 189 - DNA, 122 bp, synthetic construct
  source 1..122: mol_type = other DNA
atacaggcctgattgtttttggagtggatgcaggccgcatacgaatagctctttctgaaacgtaatgttctttgcacggctcggtggctaacgtacaataaatcaacaaaagagcgcttcaa

SEQ ID NO: 190 - DNA, 122 bp, synthetic construct
  source 1..122: mol_type = other DNA
tgccattgtagccgtagccgtacctgccggagtgcatgctcgccgagtccctgaattgctcgctcacggaactatgatctccataattatgcaactggtagtccgggccatttggatagcga

SEQ ID NO: 191 - DNA, 122 bp, synthetic construct
  source 1..122: mol_type = other DNA
gcctagttacattacgagttccctatcacccttaaaagtttttcaagcaatatatatgtacgttaccattcaattatgcacagccagcctttgatccagaaaaaagaattactttaagccgc

SEQ ID NO: 192 - DNA, 122 bp, synthetic construct
  source 1..122: mol_type = other DNA
tttaaaaattttaaagcagcagcagttaccatgcaattcaggctaattctgcgtaggctccgaacggatataattatcgaggagtcaagatgttatgctaaaaactatgcattgatattccc

SEQ ID NO: 193 - DNA, 122 bp, synthetic construct
  source 1..122: mol_type = other DNA
aaggcagccatcataaaccactttatggcagaaacccccgtctataaatcctgcgacacccggagacagcttcagatttataaaactgcttgttaagccagggcactgcttaatgaaaagag

SEQ ID NO: 194 - DNA, 122 bp, synthetic construct
  source 1..122: mol_type = other DNA
caattggctataaccatgtcatcatcacaactggtctcacaaaaaacatggggtgtctggcgacagctaaatcacagactggcgtctcctaagcagtctgtctaccattagggtctgggaaa

SEQ ID NO: 195 - DNA, 122 bp, synthetic construct
  source 1..122: mol_type = other DNA
cacataaaaggtgacggatggttaataggggagataacaggattcataagggaaatttatcggtcactctggtggtataaatatgtttatagcggaccaaggggaaatgaagaagccccaat

SEQ ID NO: 196 - DNA, 122 bp, synthetic construct
  source 1..122: mol_type = other DNA
ttattccgtgccacctgatccagccgggccttgtttatgagttagtgtgttagcagatgccgatggaagcatttgcttcgaccttgctcttctcattggggcatgaaattacgacggcagca

SEQ ID NO: 197 - DNA, 122 bp, synthetic construct
  source 1..122: mol_type = other DNA
gcgccagcctccccttgttgcacccccaccctgcctgtccccccaccactgtcattgtgtcgggctaatgaggtaaactgaagcgagccctgtcccccctgacaggtttatgagaatttaat

SEQ ID NO: 198 - DNA, 122 bp, synthetic construct
  source 1..122: mol_type = other DNA
tttgctgtcgccactttattgaaaatagcctgtaagttgttattaaatgtattgactgaacgacagggagagtagtaaaactgccccttaagatggcttttaataggaagcccgagaaacaa

SEQ ID NO: 199 - DNA, 122 bp, synthetic construct
  source 1..122: mol_type = other DNA
catctgttgtgtactttagggatataatactcgtcaaaaactccatcgtattatgagaatcggcaccaagttgatctgaaatgcatatttgaacagatgtgtttaaattacatctccgtcta

SEQ ID NO: 200 - DNA, 122 bp, synthetic construct
  source 1..122: mol_type = other DNA
cattcggaagtgcttcatgccctgcttctgtacagcagatgctgtgcattaattggcctgcgtagaagtgacccagggttccttgcagcgggcccaattttaggcagagggtttaaatagct

SEQ ID NO: 201 - DNA, 122 bp, synthetic construct
  source 1..122: mol_type = other DNA
catgaagtatagctgttgatcttacagatatcaaattacaatatatccatgctgtgcttacgaaggttttactgagagaacaacaggagctgtcttgattattgtctgcagggttattgcaa

SEQ ID NO: 202 - DNA, 122 bp, synthetic construct
  source 1..122: mol_type = other DNA
ttgccggcttcttcaggtgcattaaggagttgggggggtgggggctacatctggcttctgcgtgtctgtttgcagcacccctggggcgagctgtggcttcctgtaacatgtaaaatatgagt

SEQ ID NO: 203 - DNA, 122 bp, synthetic construct
  source 1..122: mol_type = other DNA
gtcaagtatattttattcctgtcatttataagcacttgtgttggctggtatttacaatgtcgggattctgtcactttggcaaccttcagagacaacatttttccagtgttacagtgacattt

SEQ ID NO: 204 - DNA, 122 bp, synthetic construct
  source 1..122: mol_type = other DNA
aaggaggataaaattaaaagtagcaccattgaagcagtggaagaaagcttgcctagatgtcggtgaactgtgaaatataaaaattgcataccaatacatttttatggaaaggaaacaaggaa

SEQ ID NO: 205 - DNA, 122 bp, synthetic construct
  source 1..122: mol_type = other DNA
tgctaacagtaattataatagactcaacaataaatggtaaagcagggcagagctgcatctcgggaattctctagggcatgtggattagctcggaaagcctgaatgattaaaatacactaatt

SEQ ID NO: 206 - DNA, 122 bp, synthetic construct
  source 1..122: mol_type = other DNA
ctctttccctactcagcgtcaacattagtgtgaccttttcctgctaattggaaaatcgtacggaaataaaccctgtcagaaaatgagaagtgactccccctttggatggccaaacggcctca

SEQ ID NO: 207 - DNA, 122 bp, synthetic construct
  source 1..122: mol_type = other DNA
caaattagttcggctgccagcgatagttttatacaaagatgtaaattatgttgtcaaaatcggttgctttctagcttgctgctcagctaaaaagaaaccactttggctgataaagatgaaca

SEQ ID NO: 208 - DNA, 122 bp, synthetic construct
  source 1..122: mol_type = other DNA
caggcagtcactcatgcattaacaaccaccaggccccaaaggtcgtttctatgacaaccccggggtccttaatgaccctggcaggccaatgattttgttttgtttgagagtgaaggaataat

SEQ ID NO: 209 - DNA, 122 bp, synthetic construct
  source 1..122: mol_type = other DNA
tctggagaaacatatttataggaggggaagggggagaaacctggcaagaatccccatgcacgtctgtgtgcagttaacctctgttgttaacactgaaggaaatgccacccagtaaatggagg

SEQ ID NO: 210 - DNA, 122 bp, synthetic construct
  source 1..122: mol_type = other DNA
atgctactgcagaataatgcaaggtttgttatgaattaaatattagaaaccttaacacagcggccaataagaattatttgccttattaaagcagcagaagacgctgattaaattttcattgc

SEQ ID NO: 211 - DNA, 122 bp, synthetic construct
  source 1..122: mol_type = other DNA
ttatgcagaaattagaataagacaagccagatggaatgaataattcatgaagtccaattacgttttatttctggtgtactttcaaacttgcctctgacttctgtgctattttgatgtgacaa

SEQ ID NO: 212 - DNA, 122 bp, synthetic construct
  source 1..122: mol_type = other DNA
attgttatcagcgtggtgtaatttgactgttgacaaagtatccgtggcagtgcgaatgagcgcagttagaagtgtggcggattgtaatcaagaagatgcttagctgtgcaacactgcatctc

SEQ ID NO: 213 - DNA, 122 bp, synthetic construct
  source 1..122: mol_type = other DNA
gacacagtgccaacgccacagtgctggagaccgcagtgagcattgctcactcgcagcgggcggatgaaaaggcagtgcagatgtaatttgttttcaatggctgtttcaaaagaataattgtg

SEQ ID NO: 214 - DNA, 122 bp, synthetic construct
  source 1..122: mol_type = other DNA
tttatggaaaagctaattcctgatctttgcatctattttagtaatagttatgcacgagtgcgagctgtggtgatgacccgagatgactcaagtggtggatggttaccacttggagggagtgg

SEQ ID NO: 215 - DNA, 122 bp, synthetic construct
  source 1..122: mol_type = other DNA
tcatctggcactgcccacaggtgaggaggtcatgatccccttctggagctcccaacgggccgtggtctggttcatcatctgtaagaatggcttcaagaggctcggctgtggttggagaaaca

SEQ ID NO: 216 - DNA, 122 bp, synthetic construct
  source 1..122: mol_type = other DNA
agttctataatggctatttttcttcatagcatctcaacagaacgccaccgtgctgaagttcggagagcagtaaacgatgaacggttaacaacaattgcacataagtaagccattagtcgtac

SEQ ID NO: 217 - DNA, 122 bp, synthetic construct
  source 1..122: mol_type = other DNA
agagttcataatgatgaataagattaagcttggagcttaaccttttgaaactgggtgacgcgttaactcggctggaacgctggttaatttaacagcaggagttgaattaacttgtcacagct

SEQ ID NO: 218 - DNA, 122 bp, synthetic construct
  source 1..122: mol_type = other DNA
aacagtaattgtttggcttggcctgcctttgtgtgaccgccagctggcaatgactggaaccgtgacacatgacactccagctgtgtccacatgtttctgcctcgcagtatccacttatataa

SEQ ID NO: 219 - DNA, 122 bp, synthetic construct
  source 1..122: mol_type = other DNA
ggtggacctgagcggaggctgggacgccctggtgggccccgggccctggaaggcgggtcccggtggccggtggcccagaatgaggccagctcccagcatgccctgcagccggacgccagccc

SEQ ID NO: 220 - DNA, 122 bp, synthetic construct
  source 1..122: mol_type = other DNA
aaccctataatgactgaaatgacccctccactctatttttgtgttgttttgcacagactccggaaaagtgaaggctgccaatctgagtagtactcaaatgtgaggaactgctggtcttggat

SEQ ID NO: 221 - DNA, 122 bp, synthetic construct
  source 1..122: mol_type = other DNA
actgcagttgctgggttgtcggcacgacaacaaagaattcatttttcataacatcagccgcgtatgcctcagcaatccttcattaatcagttacattatggggccgttcctccgtaatgtgt

SEQ ID NO: 222 - DNA, 122 bp, synthetic construct
  source 1..122: mol_type = other DNA
gttgactagaacagcatattttgaattagcttgcttctttttaagctaacttttctcaagcgttaaactgatgaactgggcattttctatgatcaatgggtaactgctggagctacagtaat

SEQ ID NO: 223 - DNA, 122 bp, synthetic construct
  source 1..122: mol_type = other DNA
ttgacagtttgaatggccaggcggcacacgtagcctgcaaaagagtcaaatggagtccagcgttagtgagattatatgttatgtggtatataatgttggatgtcaactccccaaaaccataa

SEQ ID NO: 224 - DNA, 122 bp, synthetic construct
  source 1..122: mol_type = other DNA
tctaattgtatagtaaataccatttcccagaccctaatggtagacagactgcttaggagacgccagtctgtgatttagctgtcgccagacaccccatgttttttgtgagaccagttgtgatg

SEQ ID NO: 225 - DNA, 122 bp, synthetic construct
  source 1..122: mol_type = other DNA
tcagacaaaaaattagtattttgcattacaagaggataattaggcaataagatacaaaagcgagtgtggttatcatgagataaacacacacatggggtgttatgaagcacagccaagtgtct

SEQ ID NO: 226 - DNA, 122 bp, synthetic construct
  source 1..122: mol_type = other DNA
cagtaatgagaacaaggctgggtccagctgtggcagccgcagccctaacgagattacacgcggcctttgatcaggacacagagcccacgtccgagccttttattactgtctccgggcgacat

SEQ ID NO: 227 - DNA, 122 bp, synthetic construct
  source 1..122: mol_type = other DNA
tgtttgtcagtttgttggacatttgagaccccaggaaatcccctttctcgtaacgttctccgcttggatctgatctcaacagggtgtcgtagtcattcttcagcacaattcttaattggaga

SEQ ID NO: 228 - DNA, 122 bp, synthetic construct
  source 1..122: mol_type = other DNA
atgacacataagaaccatccaaagtgcaaggggattgacccctgctccgcagcactctttcggcttaataggaacagctggtcagtgaagtgtttgtgagcaatttaacaagactcaaacag

SEQ ID NO: 229 - DNA, 122 bp, synthetic construct
  source 1..122: mol_type = other DNA
tttttcttctacatgatttatgtgagttgagtataaaactttttatgggagtgcagttaccgaagatgagaggtaaattaatgcactgggtttccatctcagcttcatgcacaattgtcaga

SEQ ID NO: 230 - DNA, 122 bp, synthetic construct
  source 1..122: mol_type = other DNA
tcataaagcctgtgaggaggccgctccccattgagtggctgtggcccatgaagttgctggcgtgctgtgcagtgttttgatctcggcagtggcagttatgttcctgataaatttgtaacccc

SEQ ID NO: 231 - DNA, 122 bp, synthetic construct
  source 1..122: mol_type = other DNA
gttgtaaataaaatgcaagtcaccacctccccccaatcctctgcatcctcgccgggcgcgcgatcggcagctgacggcctaacaattggtacatcctaatggaactgcgagggaaatgcaat

SEQ ID NO: 232 - DNA, 122 bp, synthetic construct
  source 1..122: mol_type = other DNA
gagagcacacccctggagaaccgactgtgcttgcttacgtttggttcatgacttaaaaatcgagtacaggtgatgaaatcttggcagtgttaacaaaaaagtagtgtgtattgtgctatttt

SEQ ID NO: 233 - DNA, 122 bp, synthetic construct
  source 1..122: mol_type = other DNA
aaggagcagacataaaaattccaatagttgtttaagcattaaagttcatttgcatcacagcgaacctgaaaagggctgacctctcaaaccgcctctcaggtctatgaagttgtagccttgac

SEQ ID NO: 234 - DNA, 122 bp, synthetic construct
  source 1..122: mol_type = other DNA
gctaaggagccgttatgtagtgccgtgtcagtgatccgtgcgttgaggactccccgagtacgcattgacacgatagaccggaacagctcagaagaagctgttatggtctatcatgccagtga

SEQ ID NO: 235 - DNA, 122 bp, synthetic construct
  source 1..122: mol_type = other DNA
acctgtttgtgggccagttgaaaagttgtctcaagtgccaggcctgtgggtatcgctccacgaccttcgaggttttttgtgacctgtccctgcccatccccaaggtgggattccagaggatt

SEQ ID NO: 236 - DNA, 122 bp, synthetic construct
  source 1..122: mol_type = other DNA
ggcgggcagggggctccgcgcggggctgcgccgctgtccggggtaatttttcatctcggccggctaatctttgttcccggcgaagataatgaatagccagtcgttatctgcccggctcccgg

SEQ ID NO: 237 - DNA, 122 bp, synthetic construct
  source 1..122: mol_type = other DNA
tgatccgtggcttcagcggctaatattggggtttctgggtgtccttgcagccacagtacacgaacctggggctcctgaacagcatggaccagcagattcagaacggctcctcgtccaccagt

SEQ ID NO: 238 - DNA, 122 bp, synthetic construct
  source 1..122: mol_type = other DNA
taccataaactgtggttgttggagatgcagcccagagaaattgtaattacagtgacaagacgaatcagcgatatttaaatattcatgaacaaagtctggggtgatcaatctatctttattgt

SEQ ID NO: 239 - DNA, 122 bp, synthetic construct
  source 1..122: mol_type = other DNA
cttccacaccctcaccctacccctacatcggacacccccaaggtgagcagcctggccaggcgcctggatttccaggaggagccgatgacaggattcgtgagttctcattagctgggggaatt

SEQ ID NO: 240 - DNA, 122 bp, synthetic construct
  source 1..122: mol_type = other DNA
gaaaaggggattaataaagcacagggccccgttggtctggctgcactccagctctgaaggcgtcctggttttgatagaatgcactccatttataaggcagagctgacgcagggaggcaggac

SEQ ID NO: 241 - DNA, 122 bp, synthetic construct
  source 1..122: mol_type = other DNA
gcacactcacgccgtttgacccgtaggcggtgtttacggtgacacggcgcttccgagcaccgtcccacagctgtctgaaagactctgcccggttgacttctgcgtaccaccactggatctct

SEQ ID NO: 242 - DNA, 122 bp, synthetic construct
  source 1..122: mol_type = other DNA
agccaggctgcggggggaggaaaaagaggttactgctactttaaatgtactgtatgaaggcgagggctggaaaggggcctgcttgcaggaatacccagtcatctagttggaaaagccgccag

SEQ ID NO: 243 - DNA, 122 bp, synthetic construct
  source 1..122: mol_type = other DNA
agcaggagggtgccgagccctccgaggaactggggttttcttcggaatgttttaatgaggcgtcaagtagcctccctcagattcctgttgtgggccttctggggacactccctggtccagcc

SEQ ID NO: 244 - DNA, 122 bp, synthetic construct
  source 1..122: mol_type = other DNA
tgtacagcagatgctgtgcattaattggcctgcgtagaagtgacccagggttccttgcagcgggcccaattttaggcagagggtttaaatagcttatttattattttgtataatctggcgta

SEQ ID NO: 245 - DNA, 122 bp, synthetic construct
  source 1..122: mol_type = other DNA
cctgggtgagggatgaaggggctggtggtgggggtgcgaggcccgaggcggctcccgaggcggccggcgcagcttagagaaatcaaaacaactggttaaaccccagcccaggggggcgtggt

SEQ ID NO: 246 - DNA, 122 bp, synthetic construct
  source 1..122: mol_type = other DNA
aagatccagttctttgattgtgactccaatgtgatgtggctggacgcacaggccggggttcgagcactgaggcgacttgtagagccgctccccatcagtactttccagggggatccccttaa

SEQ ID NO: 247 - DNA, 122 bp, synthetic construct
  source 1..122: mol_type = other DNA
agggctggagagcagctcagctcgctagctgcgcgcttcccggcacaggcagtgccactgcgcaggttgatcagcgaaacagcatccattttaatctgcggggagcccctgccttaccaggg

SEQ ID NO: 248 - DNA, 122 bp, synthetic construct
  source 1..122: mol_type = other DNA
agttccctcatgactagtgcttggctgcttgccaagcggtctaaatctgtagttgtaaatcgtgagttgaaatagcagaaagtggatttgtaacttaaaaagtgtaaacagtttggaaaatg

SEQ ID NO: 249 - DNA, 122 bp, synthetic construct
  source 1..122: mol_type = other DNA
ccctgctgcgtgctctccaaccccgaccagaagggcaagatccggcggattgactgcctgcgccaggctgacaaggtgtggcggctggacctggtcatggtgattttgtttaaggggatccc

SEQ ID NO: 250 - DNA, 122 bp, synthetic construct
  source 1..122: mol_type = other DNA
ccacaaggctacagctgccactgtcgcctgggtttccggccagcggaggatgatccgcaccgctgtgtggacacagatgagtgccagattgccggtgtgtgccagcagatgtgtgtcaacta

SEQ ID NO: 251 - DNA, 122 bp, synthetic construct
  source 1..122: mol_type = other DNA
aggcacattcttgggggaaaaaaatgtatttttttctgcattccagctgcctgagctaagcggaggaatgaatggtttggggaaaatctcacacaggaataaatttacaagggacagctgag

SEQ ID NO: 252 - DNA, 122 bp, synthetic construct
  source 1..122: mol_type = other DNA
catttaaacgagttgctccaagacaatctgattatacggtagttactttggtgagaatagcgcctcagtggaatggaaacttcattttgagtctaatattagaccttgctttggaaaattcc

SEQ ID NO: 253 - DNA, 122 bp, synthetic construct
  source 1..122: mol_type = other DNA
ggggaaccccgcaggagaccagggccctgaactcaggggcttcgccactgattgtccaaacgcaattcttgtacgagtctgcggccaaccgagaattgtggctggacatctgtggctgagct

SEQ ID NO: 254 - DNA, 122 bp, synthetic construct
  source 1..122: mol_type = other DNA
ctggctgaagcttatagctggtgtttgttacaaagtacccaagcttttcctttccaacttcgtggctttgaagaattataggaattttatgatgtgctgttggagttatactggcagagatt

SEQ ID NO: 255 - DNA, 122 bp, synthetic construct
  source 1..122: mol_type = other DNA
ggctcttgtataatttatttaactctgtgctctttgccaaagcctggaatatttttagctcgttctgttgttggatttggcttttttatgtgtgtttctgaggggtatttttagcctcatcc

SEQ ID NO: 256 - DNA, 122 bp, synthetic construct
  source 1..122: mol_type = other DNA
ccgccgcgaccccagcacagcgcctaagccccagctttgatgtccaagaacagaaaacggcgattaaatgttgttttaaagagggagtgctcactgccctcccatctgtcaagagaaagctg

SEQ ID NO: 257 - DNA, 122 bp, synthetic construct
  source 1..122: mol_type = other DNA
cgcggtgccgtagaaggcgccggccgtgaagccgcggccgaacgggcggccggaggtggacggcttggcaaagtccgagtccttgctgtccaaagatggactccgatggatcgaagaatcct

SEQ ID NO: 258 - DNA, 122 bp, synthetic construct
  source 1..122: mol_type = other DNA
ccaagcaattgaggttattatggggcagggcagacagacgggacagtggggttactagaccgtagcatttttagaaatacaggcctgacctacttagagttattctctctggctggcagctg

SEQ ID NO: 259 - DNA, 122 bp, synthetic construct
  source 1..122: mol_type = other DNA
ttaaacattttggtatatcacctagagacagtcactttctttcactgaagatcaagttatcggcaaagtacaaaatggtaattaaaaatttctagttcagtgcgaggcacacattgttcttt

SEQ ID NO: 260 - DNA, 122 bp, synthetic construct
  source 1..122: mol_type = other DNA
tcagtttcagcagtggaaagcaggcaatggagtttatgttgttcttctgtttattaaagacggatgggagcactcgacagaaaaatcaaacttctggtcatggctcattaatccctgttgta

SEQ ID NO: 261 - DNA, 122 bp, synthetic construct
  source 1..122: mol_type = other DNA
aagtggtgcagaaatcctttgcacaagatgatagggaggcctccatgctgacactgtaggcgagccaagttttcatgtacttggatggctgacctccaacttctgacatattcattatattc

SEQ ID NO: 262 - DNA, 122 bp, synthetic construct
  source 1..122: mol_type = other DNA
cgaggcccacctgaagtgctttgtgctgtactgctgaggggaacgttgtagttggaggcacgaatggatgaccaggcactagttgaaggcagcgtggtgttcaaggatgaggatgaccctgt

SEQ ID NO: 263 - DNA, 122 bp, synthetic construct
  source 1..122: mol_type = other DNA
ttggttttattcattttccttttcattgctgagagctatttttctttctttcagcttggtcgggaggaacctgccatgtccatggatgccaatggaaagataatttgggccaagcattcaga

SEQ ID NO: 264 - DNA, 122 bp, synthetic construct
  source 1..122: mol_type = other DNA
accatgctgccctcgcaggaggcctccaagctctaccacgagcactacatgcggaactcgcgggccatcggcgtgctgtgggccatcttcaccatctgcttcgccatcatcaacgtggtggt

SEQ ID NO: 265 - DNA, 122 bp, synthetic construct
  source 1..122: mol_type = other DNA
agaggatgcagatttcaaaagcctaaaatctgcaatgaagggaaatggaaatgtgctgaacgtgggtttgactggcagctttcctcatctgtcgatcacagggtaataaacactgggagatg

SEQ ID NO: 266 - DNA, 122 bp, synthetic construct
  source 1..122: mol_type = other DNA
tgccaggaaatattacgtttgtaccaatcaatagtgtcgatctagattctttgggactctcgggcagttctcagacaatgactgcaggcattaatgccgacggacatttgataaacacagga

SEQ ID NO: 267 - DNA, 122 bp, synthetic construct
  source 1..122: mol_type = other DNA
ttcttcatattttattatgttcttgtctgcagaatctgtgtcaggcaaaaaccttgtcagcggaaaacctgtcgtaggatcagaggagacaaaaacaggtgtcacacaggcagtcaaaactg

SEQ ID NO: 268 - DNA, 122 bp, synthetic construct
  source 1..122: mol_type = other DNA
ccattatttaatcattaatttcctcctctcgtcgaccccttccaccccgcctcagctcggcgcccccgccccagattagatctccacgtggtgctttgcagtgatccttgacagggcttctg

SEQ ID NO: 269 - DNA, 122 bp, synthetic construct
  source 1..122: mol_type = other DNA
cccttgtttacagtgaggactcggccactgtgtctctgtttcctgaaatataaactgtagcgaccccagactgtagagatttttatgtgtttggatacatctgctgtgtggaaaaaaaaaaa

SEQ ID NO: 270 - DNA, 122 bp, synthetic construct
  source 1..122: mol_type = other DNA
ggggaaatgaatattaatttatcatgattaggcaaatccggcagggagagccgattccggcggaggaggctaaaaagatgaatttcagcaataaaattaaataaggacggacgggcagcccc

SEQ ID NO: 271 - DNA, 122 bp, synthetic construct
  source 1..122: mol_type = other DNA
catcagcgctcggatctgcggtggtaaatgatgcattcgatggcgggcgcatttgttgtgcgctctctgaaagggctccgataatctggaaggcagagataaggaacaccatttattccgcg

SEQ ID NO: 272 - DNA, 122 bp, synthetic construct
  source 1..122: mol_type = other DNA
gtgcactttaggtttatttgcacactctttttagaaagagtagggggttgggattcaaggcggatgaaagagttatttatgaacagacgggacaccaggatggggtggtaagaaagctatgg

SEQ ID NO: 273 - DNA, 122 bp, synthetic construct
  source 1..122: mol_type = other DNA
ctgtttaaacagggcccagttgtgaagtgccctgacttcgcacacaccaaggcccggcaacggctgcaataaatcaataggaaaatagaagctgccagttagactaaacaagtgcatttgca

SEQ ID NO: 274 - DNA, 122 bp, synthetic construct
  source 1..122: mol_type = other DNA
actttaaaggaaaatggattttttttttgagcgtactttaatgaagaagataaagcggcccggctggtgtcccacaggctcggcgcagtaagttcagattattgctcagtctgtcagaatgg

SEQ ID NO: 275 - DNA, 122 bp, synthetic construct
  source 1..122: mol_type = other DNA
acgcccatgccccctccgtctgtgcgcgccctgcgcccccggcacgcgcccccctccaaccgggtgtctgacagccggacagcgggatctgcacagcaagtgaaattcccgtcggatcgata

SEQ ID NO: 276 - DNA, 122 bp, synthetic construct
  source 1..122: mol_type = other DNA
ccagtgcagctagagtccagtctcctaagatcaaaggctatgctgaatgaggtggagaagcgccgctccctgctggaaatattgaatagtgctgctgacattctgatcaattcttcagaagc

SEQ ID NO: 277 - DNA, 122 bp, synthetic construct
  source 1..122: mol_type = other DNA
tgaaacgcaattagccctctgtgaaaggggcccctcacagccccctcggccaatcccctccgaggcactgctcacgttacaatggccttgaccacaatccattgagtagaaaaaggaaaaaa

SEQ ID NO: 278 - DNA, 122 bp, synthetic construct
  source 1..122: mol_type = other DNA
atttgagctgtggaaatgctggaatgtgtttttgtgcttttcctgaacaagggaattcaacgggtaaattacagtcatttactgggagtgtaatttactgagagcctttaggcaaatgcagg

SEQ ID NO: 279 - DNA, 122 bp, synthetic construct
  source 1..122: mol_type = other DNA
ccgggcaggtgaggacaaacgaagaagtgtacgaagggttaacaacggggaaggggtcgccgaggacttgaacttcactgtgtgtaaagagagaagccgtcaggttggggggtgcgtcttgg

SEQ ID NO: 280 - DNA, 122 bp, synthetic construct
  source 1..122: mol_type = other DNA
aaccactgacatatggatgtgatctctgttactttgaccagattcggtttctacagagtacgtgagaagggaagataaattttgtcctagtaatactatagcgactcataaataaatggtta

SEQ ID NO: 281 - DNA, 122 bp, synthetic construct
  source 1..122: mol_type = other DNA
cctcagaaactatttaactttcattaaactgtgtttgatatcagctaaataggatgcatacggctccaatatagctcatatggtcgcattcattcgatcacagcagtgcttttttcttatat

SEQ ID NO: 282 - DNA, 122 bp, synthetic construct
  source 1..122: mol_type = other DNA
cagcctgcttgatgctggcatcggcttgcttcgtgcttaaaccgcttattaaagtctagacgcaacataaaagcacgtaaattggttccagccatctctagtaaaaacttcagctggagctg

SEQ ID NO: 283 - DNA, 122 bp, synthetic construct
  source 1..122: mol_type = other DNA
ttgtttaccagcgacattcgcagaaaaaggcgacagctgggacagaaagctgctactcaacgaacaatataatcccgtgaaagtaatcaacaaatttaagggtgttaattaactttacactt

SEQ ID NO: 284 - DNA, 122 bp, synthetic construct
  source 1..122: mol_type = other DNA
cagggaaccaaagccaggagagttcagcaaggtgatcaggagactcatggagacgcccaacgcccggggcatcatcatctttgccaatgaggatgacatcaggtgggacagtggcacaatca

SEQ ID NO: 285 - DNA, 122 bp, synthetic construct
  source 1..122: mol_type = other DNA
aaatgagtttacaaaataagagctcatttgttttttgatatgtgtgcttgatttgtggctcgcggtcgtttgtgcgtctatagcacccttgcacaatttatgatgaattatggaaatgactg

SEQ ID NO: 286 - DNA, 122 bp, synthetic construct
  source 1..122: mol_type = other DNA
accgcaggctttcaccttgcagataatggggtgagcggcggtaaactctccccgactgagcgggctcccccggccttaatgagataatcaatacttgttctcggcttaatttccatctaatt

SEQ ID NO: 287 - DNA, 122 bp, synthetic construct
  source 1..122: mol_type = other DNA
taatttgaattgataataggaaacgcggggaaaagtcatctatggtgagtgaggagcgagcgatccctgagcagatgctaaatcttgtataatgaaagaatctgccgatgtcaattatgtac

SEQ ID NO: 288 - DNA, 122 bp, synthetic construct
  source 1..122: mol_type = other DNA
gggccgccaggatccctccccgcggcggcgatggcccgagccatgagagtgaggactttccgcgcccattggtgacccttccaggcagacagcctcagcaacgcccctggtggacaggatgg

SEQ ID NO: 289 - DNA, 122 bp, synthetic construct
  source 1..122: mol_type = other DNA
tccaattacagagggtggaaacagccttcaatgacaatatgacaaggacagaacatagaacgctggcacagagaacacaattatgcacagccttgaaaaattgtatcatagcacaatgcatg

SEQ ID NO: 290 - DNA, 122 bp, synthetic construct
  source 1..122: mol_type = other DNA
caacttccagtaaaagggagacgggggaacgggagagaggaaaaaagcattctgggaggtcgcggagggcagagcagtgacctccaatgatttacaggcctttagcttaatgaaattgtttc

SEQ ID NO: 291 - DNA, 122 bp, synthetic construct
  source 1..122: mol_type = other DNA
tcattggttatatgaagtagacaactgtggttaatactgctcttatttacagagagaaagcggttggaagccaagcaacgggaagacatctgggaaggcagagaccagtctacagtttgaac

SEQ ID NO: 292 - DNA, 122 bp, synthetic construct
  source 1..122: mol_type = other DNA
cagtgaaacaaatgaacccactggggattccttaatgaatgtttaactgcaaacggtagtcgggcacttaagcagctaggctgatggtccctaatgaaatcaaataaaatcaaataaaagaa

SEQ ID NO: 293 - DNA, 122 bp, synthetic construct
  source 1..122: mol_type = other DNA
gccctgacctgagcggtaatgaagggagagtgcacggcaaagtgtccattatcagcaggtcgggagcagtaaattggagcagtttgcttcaatcttgctgcaagttgaaatgtaagataggt

SEQ ID NO: 294 - DNA, 122 bp, synthetic construct
  source 1..122: mol_type = other DNA
ctggcgctggcagcgtagctgcgggcgcgctctccggagccaaagtggccggagcccgagcggccgacgctgagatccatgccattgtagccgtagccgtacctgccggagtgcatgctcgc

SEQ ID NO: 295 - DNA, 122 bp, synthetic construct
  source 1..122: mol_type = other DNA
gcaaatggaaagagaactgccaggactaaatttcagcctgccctcagaatagtgcaaaggcgctacaggaattgcacgacagatggtctttgaatgatatacagaacctctgcccttgggcc

SEQ ID NO: 296 - DNA, 122 bp, synthetic construct
  source 1..122: mol_type = other DNA
actctcatctgggtctgtggcggcggaaggagcgagagatcccagcactaaactctccctcgctctgttttttgaggaacctgaatgaacctgaaagcaaagaccgagtggagacgctcagg

SEQ ID NO: 297 - DNA, 122 bp, synthetic construct
  source 1..122: mol_type = other DNA
gcatttgttgtgggtgaatatgactttgtagaaacaaggtcaaaagatggtgtgtgtgtccgtgtttacactcctgttggcaaagcagagcaaggaaaatttgcgttagaggtaaatgtact

SEQ ID NO: 298 - DNA, 122 bp, synthetic construct
  source 1..122: mol_type = other DNA
ctttttgttgatgttgctttcccccttaattatatgcatgtaggttaaatgaatacctgacgaaagggcccacgtttcaaggcagtgacatttgatagctgagaggaaaagtggctttaatg

SEQ ID NO: 299 - DNA, 122 bp, synthetic construct
  source 1..122: mol_type = other DNA
cgccccagctcggggcgaggccccatccccacccccagccacccaggcctctgctctgggcggcttggggaccagccacattcttgacctcaggggaaaagtttagactctttgcgccctgg

SEQ ID NO: 300 - DNA, 122 bp, synthetic construct
  source 1..122: mol_type = other DNA
caggtgttcagcattagcaaatatttcttattatatggattgacttttggcagctagtaacgatgtttcctgagattgaaaaatgattttctaccttaaacagtggtttcacattactggaa

SEQ ID NO: 301 - DNA, 122 bp, synthetic construct
  source 1..122: mol_type = other DNA
ctctgcaatgtgggagagacagagacaccgatcaccatcgagattgtccttacttgcagacgcacaagtatcctgcatgttccgattctgactggcacctggagcggctctacatgccagtc

SEQ ID NO: 302 - DNA, 122 bp, synthetic construct
  source 1..122: mol_type = other DNA
gcacaaaagtcatcagccagtgaatgttcttttctgcaccacaattaagacgaatcaatacgacgtgccacaaatggtattttactctcaataagaactcgagctgagtttattcagaatat

SEQ ID NO: 303 - DNA, 122 bp, synthetic construct
  source 1..122: mol_type = other DNA
ttgaaatctgtgacctcagaggctgacatggtttgatgggaaatttaatgtaataggctgcgaaagccaagaggcctgttgtcagcaatgaagtctgaaagttagcagaattaataacactg

SEQ ID NO: 304 - DNA, 122 bp, synthetic construct
  source 1..122: mol_type = other DNA
gagaattgttttaaaaggcatgtaatttattagctatcggtagcttctttattgccttgccgcactgcagtaataaataaaagaaagtgaactgattttacaaatggcacacaaggtgcaca

SEQ ID NO: 305 - DNA, 122 bp, synthetic construct
  source 1..122: mol_type = other DNA
cagctgtgacttttcgccacaaggcgcaggctagacagggccctaatcagatgggccagccgggcagtggggccagtgtctttatcggccagatggtgtgaatttagacacttttgttatgc

SEQ ID NO: 306 - DNA, 122 bp, synthetic construct
  source 1..122: mol_type = other DNA
ctgtattgaaacgtgtgaccagggcatgggatcatggattgtctttcactgttgtcatggcgacagtgatgcaggtggcctataactgccgggctgtcagatcttatacatcaaactggtgc

SEQ ID NO: 307 - DNA, 122 bp, synthetic construct
  source 1..122: mol_type = other DNA
aacttgtggtttggacacttctggacctaaaattgacagtttgaatggccaggcggcacacgtagcctgcaaaagagtcaaatggagtccagcgttagtgagattatatgttatgtggtata

SEQ ID NO: 308 - DNA, 122 bp, synthetic construct
  source 1..122: mol_type = other DNA
agctctggcctggcagagagcctccctccaccccaggagctgaggtaaccctccgccccccgcgcacgcacacatacccgaccaaggcctcaactctgagcaagagctaggaaggaggtctg

SEQ ID NO: 309 - DNA, 122 bp, synthetic construct
  source 1..122: mol_type = other DNA
cactgttccagatactcattcagctcccgaatggcctccacatttttcccctgggcttttcgaatggcaatcttacgctttcttgcagcctgtgggttgacattaataaaggattaggccct

SEQ ID NO: 310 - DNA, 122 bp, synthetic construct
  source 1..122: mol_type = other DNA
caggccgtacaggggccaggcctggctgaatgggatcccccagccccaccgtttgttatccgagctgagctcacacaaattaatgaggagagggaatgaggaaagccaagggctgttgctgg

SEQ ID NO: 311 - DNA, 122 bp, synthetic construct
  source 1..122: mol_type = other DNA
ctaaactttgcttccagcgcggcttcccttgctaactctgccaaccagtgtcaaagtggacggtgggcttaacctctgctattaatctgtacaagtgcctcctgggctttgttccaagcaat

SEQ ID NO: 312 - DNA, 122 bp, synthetic construct
  source 1..122: mol_type = other DNA
caactgagcgaggacggagggaagcaaaggacagagccaggagcaagcccctggcggcggcgactgtggcttctgcccaccctacatacttcacagacaggtgcaggtcggcgggcgccctg

SEQ ID NO: 313 - DNA, 122 bp, synthetic construct
  source 1..122: mol_type = other DNA
ttgctgggttcatggtataattagctctatatttcaccaaatacaattttgctcccatggcggacaaagcagcatgaagaaaacataaataattgcagattagagaaacagaaggcaaagtg

SEQ ID NO: 314 - DNA, 122 bp, synthetic construct
  source 1..122: mol_type = other DNA
gaagactttagaaattcaaatgaggctctcgggggagagcgagtcttaaacattcccctgcgctggcggctttattgaaggaataaactatatttgcaaagtaagatattaaggctgtcagt

SEQ ID NO: 315 - DNA, 122 bp, synthetic construct
  source 1..122: mol_type = other DNA
tcacagtaaatgtgttaatgtttcttcattgcttgactgtttgtgtttaaatttaggcagcgtgcccaacagggacaaaattataggataaagctatgtataaaagaaaatccttcagagag

SEQ ID NO: 316 - DNA, 122 bp, synthetic construct
  source 1..122: mol_type = other DNA
aagctcattagcattctgacaaaattggagtggtggagatgtatagtttgcctagtttgtcgttttgaaagctaccggtggtaagctttctatttcattgtgcctttgtcttttgggggggt

SEQ ID NO: 317 - DNA, 122 bp, synthetic construct
  source 1..122: mol_type = other DNA
gtgtcacagaccggcgatcagacacttttagatggtccggcccatgactttgaccttctgcgcagtccggcgttccctggggaaccgttcaactcatgaggtctgatttcacacttccagga

SEQ ID NO: 318 - DNA, 122 bp, synthetic construct
  source 1..122: mol_type = other DNA
caggcctcccacccagccctggtgccctggcctggcccactgtggccacataaaaggtgacggatggttaataggggagataacaggattcataagggaaatttatcggtcactctggtggt

SEQ ID NO: 319 - DNA, 122 bp, synthetic construct
  source 1..122: mol_type = other DNA
caaataaatggagtaaagacaggagattagtagaatgagcagaaatgagctgaaccgctacggttcgcagctaatgagcatgcagctggattgcaaatggctggatttatagtgttggtata

SEQ ID NO: 320 - DNA, 122 bp, synthetic construct
  source 1..122: mol_type = other DNA
ttcatggtgcagcggacattgcagaactcgcacttgaaaggcttctcccccgagtgcacccgcatgtgccttttcaagtccgagctgattttgaacttggcgctacagagccagcactggaa

SEQ ID NO: 321 - DNA, 122 bp, synthetic construct
  source 1..122: mol_type = other DNA
tgttttctcttggttcaggaggccccttcgacacctgctttatttttcttcctgcgctgacgtgacgggagacaggtgttaaactacttaatcactttaaaattgttttaattttctgtttt

SEQ ID NO: 322 - DNA, 122 bp, synthetic construct
  source 1..122: mol_type = other DNA
ttttaatttggctgagcgccttttttcctacctccaagagctgttaattttgttcgctgccgagcaaaagtcaaagtcagagtctaaacagcttgtaatgagttttcaatggcccatctcaa

SEQ ID NO: 323 - DNA, 122 bp, synthetic construct
  source 1..122: mol_type = other DNA
gtttttgtttttgtttttttatcaaatcctgcctgactgtctgcttgttttgcctaccatcgtgacatctccatggctgtaccaccttgtcgggtagcttatcagactgatgttgactgttg

SEQ ID NO: 324 - DNA, 122 bp, synthetic construct
  source 1..122: mol_type = other DNA
atgatctccatctatgtgggcaaatctcatcttggcctcatctgcggctttcttggcctccgtggggcgaacaaaacactgtgggactaaacaaggttggtatgggcccaacacaaacgccc

SEQ ID NO: 325 - DNA, 122 bp, synthetic construct
  source 1..122: mol_type = other DNA
gagctcttccattttagcttccattatccaccacccaagttctcaattcaatttcagactcgggcaaaagcctgtaattctatttgttttgggtaaatttagcaagctgctcctgattacac

SEQ ID NO: 326 - DNA, 122 bp, synthetic construct
  source 1..122: mol_type = other DNA
ggcttcacgtggtcctgttcgggggaggcaatgaggcaccggcggctctgttgctgctcccggagattaatgaggcctaagagtgcattaggaacagtgacaaaggtttgatctaatgggct

SEQ ID NO: 327 - DNA, 122 bp, synthetic construct
  source 1..122: mol_type = other DNA
catgatggaggagatgtgcgcgctcagcttcagccaagagctcgactactggggcatcgacgagatctacctggagtcctgctgccaggcccgctaccaccagaagaaagagcagatgaacg

SEQ ID NO: 328 - DNA, 122 bp, synthetic construct
  source 1..122: mol_type = other DNA
atacttaaagctgcctttccaatgcaaattttataggaaatctcaaccagctaggacagacgtcctagctgtcagcaggcaaatgctttcattttggtctcaggcaagggagctgcctgctt

SEQ ID NO: 329 - DNA, 122 bp, synthetic construct
  source 1..122: mol_type = other DNA
tttttgtgatggtccccatggtgacaatttgtgacggcaaagaatgtgggaacggggcgccgccgcctgattgggatgctttgtatctggagaggcgctcctgattggcctgaggggccccc

SEQ ID NO: 330 - DNA, 122 bp, synthetic construct
  source 1..122: mol_type = other DNA
tgtaggtattcagactgatcacacaagcccagcagaagtcccggaccatccgcattctgtcgtatacagttgtttcatgtggcctgtgatttacagtcttaattaatgctcaaaagaagaag

SEQ ID NO: 331 - DNA, 122 bp, synthetic construct
  source 1..122: mol_type = other DNA
gagttggcgctggtttgtgccgggagggagcaatatgttctgacaactggtgctgggctgcgtttacccaacagccagcctgcctggggagacaatatttgaaaaacacagtattgaaactt

SEQ ID NO: 332 - DNA, 122 bp, synthetic construct
  source 1..122: mol_type = other DNA
tgcaatatgaaagtggattggcttctccctcccttggggcccctgtatctcttcatccagcgccttgggtaaaaagcaatgcatacgtttacaatataaatgtgatttaatcgacctccctg

SEQ ID NO: 333 - DNA, 122 bp, synthetic construct
  source 1..122: mol_type = other DNA
ggttaagtgctgtctcattgctttgcaggctgcctgctgtttcccggggagatcatgaaacgaggtcgccttcccagcagcagtgaggattctgacgacaatggcagtaagtcctgctttgt

SEQ ID NO: 334 - DNA, 122 bp, synthetic construct
  source 1..122: mol_type = other DNA
cctcagtattttgatattttcattggctgttaaacgtggaaactttttaacatgctttgtcgcatttatatgctacaggttacaaagtgagagccttgtatacacttcaatacttaaaaagt

SEQ ID NO: 335 - DNA, 122 bp, synthetic construct
  source 1..122: mol_type = other DNA
ctcaaaatggggcctacatctgacaggcaggaatttagttaaaagcttttaaacgggaaacgtcattttgatgtcacgtttccatagcaacagagcaactgcataagctacaacaacagaaa

SEQ ID NO: 336 - RNA, 122 nt, synthetic construct
  source 1..122: mol_type = other RNA
ttagtctattcaatgggggctacttgaaggccggagggacaaagcgtgtacacattgcaccgctattcattccggccagctggatcggctgaaaaaaaaaccttgtgaaaagaaacgcctca

SEQ ID NO: 337 - DNA, 122 bp, synthetic construct
  source 1..122: mol_type = other DNA
ttgatatgttgtctttgtagaaaagaattcaattctagacgaggtgttcgccgtcacattcgaaaagtacacaagaaaaagatggaagaactaaaaaagtacattgaaacacgaaagaatcc

SEQ ID NO: 338 - DNA, 122 bp, synthetic construct
  source 1..122: mol_type = other DNA
gcttttgaaattgatttcctttcctttctccccctattcaggacctaaacgctgaatgttcgatatttcatttgagaccgctaatcgcattactgcccttgacgttttacagatatcatatt

SEQ ID NO: 339 - DNA, 122 bp, synthetic construct
  source 1..122: mol_type = other DNA
aaaagaaggtttcaatacaaggatatttttgaatagctggcaaccacaagcactgatgtacgagccagaaggaagactcacaaagatttcaacatacttctggtaggtagattcacagtaga

SEQ ID NO: 340 - DNA, 122 bp, synthetic construct
  source 1..122: mol_type = other DNA
acactaattaccgcacagaccttctccatgaaatcggtgctgaagccatttccatccaggcggctctccaagacgaaatttttctactgggaaccgacagccgcaaataaacctaccatcta

SEQ ID NO: 341 - DNA, 122 bp, synthetic construct
  source 1..122: mol_type = other DNA
actgagccacggcatttccagctctgctacctctcacggcttcctcgctggtccctgtgacgctgtcactcaagggctttcaagtcagcgatgtagaaatcaaaaagaaaagggaaatggtc

SEQ ID NO: 342 - DNA, 122 bp, synthetic construct
  source 1..122: mol_type = other DNA
tccatggggtcggaaaaggcttgttcctgccaccagcccccaagctccagcagcccctcacgccaactgctgggtgccatttaaaccctccccgctggcatggaaccagagataaatatagc

SEQ ID NO: 343 - DNA, 122 bp, synthetic construct
  source 1..122: mol_type = other DNA
tcacggaactatgatctccataattatgcaactggtagtccgggccatttggatagcgaccgcaaaatgagtttacaaaataagagctcatttgttttttgatatgtgtgcttgatttgtgg

SEQ ID NO: 344 - DNA, 122 bp, synthetic construct
  source 1..122: mol_type = other DNA
caggtagcgctccagggtggggtttcggccctctgcctggttcagagctggctcgcaatccgaggccatgatgctcgccttgcccaactgtgaacacaagagggactgagcatccctctgca

SEQ ID NO: 345 - DNA, 122 bp, synthetic construct
  source 1..122: mol_type = other DNA
atttcctcagggaagaatgaatcgcttccctgtgtttagtccagtaactaagaagcggagcgccacttgattggtttgggatttccaggacatggagttttgaatttgcatacagactagag

SEQ ID NO: 346 - DNA, 122 bp, synthetic construct
  source 1..122: mol_type = other DNA
ttccccgctttctgttctcagtgtcctcggtcatggtgcttttcgttgcattaaaagtgccggtcaaactttgatagtatttttttatagttggtgcagagtggaataactcatggattatt

SEQ ID NO: 347 - DNA, 122 bp, synthetic construct
  source 1..122: mol_type = other DNA
gccgcttatgacggggttctaatgaagcacgactctgggagccggagacccccgtggacacggcagttcaaagggaatatttcaacaatgattacattttgtaaatctttttatgaaagaga

SEQ ID NO: 348 - DNA, 122 bp, synthetic construct
  source 1..122: mol_type = other DNA
gctgtaaagtattaactcacactgaacacagtttgtctctgaggaccctcttggccccttcgtcatcctgtgctgacattttaatcgtgcttttgtgggagctggctggcagtttgtcatca

SEQ ID NO: 349 - DNA, 122 bp, synthetic construct
  source 1..122: mol_type = other DNA
gacttcattagtgcccagatttaaaatttcacattttgtttcttgtaattatcttctgatcgactgagctcaaatttcagttggggccctattagtctgcctttccaaaaagggcgttggga

SEQ ID NO: 350 - DNA, 122 bp, synthetic construct
  source 1..122: mol_type = other DNA
agggtattttggttttgcatgaaaagacaacatccaattagcctctacaggaactcacctcggattatataaacctgtccacctatcaagtgttttagacaacagaacagtccatctgacaa

SEQ ID NO: 351 - DNA, 122 bp, synthetic construct
  source 1..122: mol_type = other DNA
ctgcccccctgcccgcctccccttgcccggcctcccgcccgctctatgaaaatttaatgccgggtaatgacagtctgggctaatgaaagaggcttctcttctggcaaaaagagaaaaacaaa

SEQ ID NO: 352 - DNA, 122 bp, synthetic construct
  source 1..122: mol_type = other DNA
tggtgcttactgtgtacctttaaaactgacttttatcatgcatttgtttcttaacagttgcgagaaagaatcaaagctggcaaaggtgtgacttcagctattgacctgtgtcgttaccatgg

SEQ ID NO: 353 - DNA, 122 bp, synthetic construct
  source 1..122: mol_type = other DNA
gaatgatgaattgaaagcagaaatgggaaaaagactgcaatgcaatgaaaatttaatcagcgtcttctgctgctttaataaggcaaataattcttattggccgctgtgttaaggtttctaat

SEQ ID NO: 354 - DNA, 122 bp, synthetic construct
  source 1..122: mol_type = other DNA
ccagaatactaatcaggaaagtcagaccctggaggtcagggagtaagtcaatcattctagcgatcgtctcagtgtggaggatcaaattagcctgaaaaattcagctgctgggaggagagggc

SEQ ID NO: 355 - DNA, 122 bp, synthetic construct
  source 1..122: mol_type = other DNA
acataaaaggcattttatacccgcaattgttcatttaatgagcaattgcagagtgcaggccggttaagggattgagctatatgcactattattgcaagaagtattccgaaataccagaaata

SEQ ID NO: 356 - DNA, 122 bp, synthetic construct
  source 1..122: mol_type = other DNA
ggaggcctccgagggcagcacgctcaccaccgtgctcttcttggtcatctgcagcttcatcgtcttggagaacctgatggttttgattgccatctggaaaaacaataaatttcacaaccgca

SEQ ID NO: 357 - DNA, 122 bp, synthetic construct
  source 1..122: mol_type = other DNA
ggaggaggaggagggaggcccagggctgttggagggaagggggaattctgaatatgtgaacgataacgaattggttctggggctaaataagggagccaggacttatgtaattaatgctataa

SEQ ID NO: 358 - DNA, 122 bp, synthetic construct
  source 1..122: mol_type = other DNA
ctctttagcctctattcagccaaaattagcctccgtggattctggctcacaaaagcaccacgggcctctggctgtgctctgtagctgtgaaatagaatcaagattccattaggcatcccaag

SEQ ID NO: 359 - DNA, 122 bp, synthetic construct
  source 1..122: mol_type = other DNA
gctacagtgcttggtggaagaaggatggcatttggctaccctgttctttctctaatgcctcggtgttgcctgtctgcgccggccattgttgcaatgtaagcaatactgtttgatgcactgcc

SEQ ID NO: 360 - DNA, 122 bp, synthetic construct
  source 1..122: mol_type = other DNA
cagatcaattagcagtcttaagtttatctgatttttatcaaagaagacagagctgtgggtcgactgtcagagcctgtgtcaagttgaccttttcagagatgtctacattgccagtcccctgg

SEQ ID NO: 361 - DNA, 122 bp, synthetic construct
  source 1..122: mol_type = other DNA
gggagagggccgcggccccctttcatctcacttagcacagaggagagacaatgggccagccgattttccttgttgtcccttgtttgaaaaggtctggtgtctctactaggtacctgtgacat

SEQ ID NO: 362 - DNA, 122 bp, synthetic construct
  source 1..122: mol_type = other DNA
tcacaaagctagttaggcagagccattcattccttcaggaaaacagggcattgatttcagcgagttcccagggagcagctgcaacaaagagatcaaaggcccacctctctttccctccattg

SEQ ID NO: 363 - DNA, 122 bp, synthetic construct
  source 1..122: mol_type = other DNA
gtatcctctggggtgccctggatgcttccgtcgggattcgcctggaggtagaaaccctggcggcagaacagtttggtgacgatgcctttgagctgaggctctggagagagagacattcatct

SEQ ID NO: 364 - DNA, 122 bp, synthetic construct
  source 1..122: mol_type = other DNA
gcgcagtgcagcattccacaagacctgctgcctctttgggcaggcgattaatctcatgatcgatagaccatatgattaggcactaatagctgataggaggagagaataaacaaccataaatt

SEQ ID NO: 365 - DNA, 122 bp, synthetic construct
  source 1..122: mol_type = other DNA
ctcagccgggtttggctgtcagaaatgtttaaatatggaaaaaaataaacagttgcaacacgtttctgtgcagagatctgcctgagctcctttgtgatgtaatgggaaacgcggcattcagg

SEQ ID NO: 366 - DNA, 122 bp, synthetic construct
  source 1..122: mol_type = other DNA
gttagttaaaaactccctccttcctcaggatccctccgcctgcggtagcggtggtgaggccgagggagccgccattttggatgttcggttcctgctgccactgctgccgccgcccccggagc

SEQ ID NO: 367 - DNA, 122 bp, synthetic construct
  source 1..122: mol_type = other DNA
cccgagcgcgccaggcctatttctgtcacacagacagattgagcctttcactcaggctcgcgagtgagtgacacatagcccgaccgggaggacaacattgttatttgagttaagaaatcaat

SEQ ID NO: 368 - DNA, 122 bp, synthetic construct
  source 1..122: mol_type = other DNA
cccgaccagaagggcaagatccggcggattgactgcctgcgccaggctgacaaggtgtggcggctggacctggtcatggtgattttgtttaaggggatccccctggaaagtactgatgggga

SEQ ID NO: 369 - DNA, 122 bp, synthetic construct
  source 1..122: mol_type = other DNA
tcccgcggctgccacccgccctcaaactaacgggccgggcaggcacaagagtctgcggggcgcgcagaggccgctctcaagccttcgagctcactccgtgctcaggccaccagcaggagcct

SEQ ID NO: 370 - DNA, 122 bp, synthetic construct
  source 1..122: mol_type = other DNA
catgcttgtaggctttcttccttttccctttcctctcctcttttcatttctaattcgaatcggaagtctatgtggagcagtctggctagccccagtctaacctccccatctggaaggggact

SEQ ID NO: 371 - DNA, 122 bp, synthetic construct
  source 1..122: mol_type = other DNA
catggaggaggccgcagggtgggccgcctgcgtcagccttgagcggccatcaatagcgcgcggttaattaatttgatgggaacagcagggaccgccgtttgcatttagtgaagttccaggaa

SEQ ID NO: 372 - DNA, 122 bp, synthetic construct
  source 1..122: mol_type = other DNA
acagttagcaggctttagctctcccttttgaatcagtgaggttgccgttttattgctctccgacttccacttcagtgatgagagtaaactagcatcagattaaaggtcagcaaatcccatca

SEQ ID NO: 373 - DNA, 122 bp, synthetic construct
  source 1..122: mol_type = other DNA
tcaatggattttggcaagagttgaaaaacccagatgcagggcttcagcctcccgctgagccgcctccagctgcgagtgtctggtggaggcactttgtaaaatcatgaaaaccctattaaatt

SEQ ID NO: 374 - DNA, 122 bp, synthetic construct
  source 1..122: mol_type = other DNA
aatccactgtaaacgccttgatgtgcagcaacagcttagagggggctcaggtttctgtggcgttggctatatttatctctggttccatgccagcggggagggtttaaatggcacccagcagt

SEQ ID NO: 375 - DNA, 122 bp, synthetic construct
  source 1..122: mol_type = other DNA
caccaagctcgaggttcctgcttcagcttgggaaataaagattagcatataaatagccttcggtgcctgcttgattccagctattttgcgtttcaatgtggcatcagaatgcacttagcaag

SEQ ID NO: 376 - DNA, 122 bp, synthetic construct
  source 1..122: mol_type = other DNA
ataaaggcagatgaaaacactccagttgaaacaccttggggcctgctcattcctctgacacggctcttttgttggaactatttagaggttattaagatgtgtcttcagaattaagagtatgc

SEQ ID NO: 377 - DNA, 122 bp, synthetic construct
  source 1..122: mol_type = other DNA
ctacctgtccggctacatctcgctgctgctgcgcgccgagccctaccccacgtcgcgctacggcagccagtgcatgcagcccaacaacattatgggcatcgagaacatctgcgagctggccg

SEQ ID NO: 378 - DNA, 50 bp, synthetic construct
  source 1..50: mol_type = other DNA
agcctctcactgctaagccggacatttcctaacactggacaaattgtacg

SEQ ID NO: 379 - DNA, 50 bp, synthetic construct
  source 1..50: mol_type = other DNA
cggctttaaagaagcgatggggtcgttaccagccgattatccagacacac

SEQ ID NO: 380 - DNA, 50 bp, synthetic construct
  source 1..50: mol_type = other DNA
caccctccctgagctggacgtacaatttgtccagtgttaggaaatgtccg

SEQ ID NO: 381 - DNA, 50 bp, synthetic construct
  source 1..50: mol_type = other DNA
cggatggaaatgcccataaagccgtatcaaaccctgtgcagagaaggaca

SEQ ID NO: 382 - DNA, 50 bp, synthetic construct
  source 1..50: mol_type = other DNA
tttaaaacgcccaacgctgccaagtcagaaaagcgtctcctgataaagcg

SEQ ID NO: 383 - DNA, 50 bp, synthetic construct
  source 1..50: mol_type = other DNA
cggtttgaatgaaacctgtgatttatgtccatttaactgaaaagccttgt

SEQ ID NO: 384 - DNA, 50 bp, synthetic construct
  source 1..50: mol_type = other DNA
cgtgattcattgtctgcaggtgattcatggttaggaaattaattctgaac

SEQ ID NO: 385 - DNA, 50 bp, synthetic construct
  source 1..50: mol_type = other DNA
cggatttcaaagcccaagacgtttcaggcctacttggatgattgtcaccg

SEQ ID NO: 386 - DNA, 50 bp, synthetic construct
  source 1..50: mol_type = other DNA
tgggaattaaatgtctgaagttcaaagcagtttgcacatcagagcaaacg

SEQ ID NO: 387 - DNA, 50 bp, synthetic construct
  source 1..50: mol_type = other DNA
cgggcccaagggcagaggttctgtatatcattcaaagaccatctgtcgtg

SEQ ID NO: 388 - DNA, 50 bp, synthetic construct
  source 1..50: mol_type = other DNA
cgcactgacctgaagttcacctcggccttggactttgcgcttctaaaagg

SEQ ID NO: 389 - DNA, 50 bp, synthetic construct
  source 1..50: mol_type = other DNA
ctggcagattaatggtgacctgtgaccctgacaggctggccacactcccg

SEQ ID NO: 390 - DNA, 50 bp, synthetic construct
  source 1..50: mol_type = other DNA
cgtgaaggaccagacagtgatgttcctaagccgcaccacctacagcctgc

SEQ ID NO: 391 - DNA, 50 bp, synthetic construct
  source 1..50: mol_type = other DNA
cggttagttacagtaattatacccaataactgacagccaaaatcacgtag

SEQ ID NO: 392 - DNA, 50 bp, synthetic construct
  source 1..50: mol_type = other DNA
cgcgccgcttgtctcttaatctccagagcgaccgatcgattcgctatttc

SEQ ID NO: 393 - DNA, 50 bp, synthetic construct
  source 1..50: mol_type = other DNA
tctaatagaaaacaatgtgcactttctaaatttgaaccaagcagtttccg

SEQ ID NO: 394 - DNA, 50 bp, synthetic construct
  source 1..50: mol_type = other DNA
ccaaagctttcctgccaccagtccttcaaatctgacttggacattacacg

SEQ ID NO: 395 - DNA, 50 bp, synthetic construct
  source 1..50: mol_type = other DNA
cgcctctttggtggtaggtttcgaccaaaagaattggaagagctatgatt

SEQ ID NO: 396 - DNA, 50 bp, synthetic construct
  source 1..50: mol_type = other DNA
cagaattagacttcttttctcaattcacagtgaattatgctgcctacacg

SEQ ID NO: 397 - DNA, 50 bp, synthetic construct
  source 1..50: mol_type = other DNA
cttttcaccctactcttttaacacatcatgtatgcactgagcaatcttcg

SEQ ID NO: 398 - DNA, 50 bp, synthetic construct
  source 1..50: mol_type = other DNA
ctttttattcacagcattgctaaatcagaagcattcacagtttccctccg

SEQ ID NO: 399 - DNA, 50 bp, synthetic construct
  source 1..50: mol_type = other DNA
actatttcaaaagcccggaatatgatttgaccaggactgagagccactcg

SEQ ID NO: 400 - DNA, 50 bp, synthetic construct
  source 1..50: mol_type = other DNA
cgtgggctgttacgttccgtttcaaacctttccctgtggcacagagagag

SEQ ID NO: 401 - DNA, 50 bp, synthetic construct
  source 1..50: mol_type = other DNA
cgtctcagtgtggaggatcaaattagcctgaaaaattcagctgctgggag

SEQ ID NO: 402 - DNA, 50 bp, synthetic construct
  source 1..50: mol_type = other DNA
gataaagatccagttctttgattgtgactccaatgtgatgtggctggacg

SEQ ID NO: 403 - DNA, 50 bp, synthetic construct
  source 1..50: mol_type = other DNA
cagccaggagtcttttcttctttgctgatttttttcaatctgtattgtcg

SEQ ID NO: 404 - DNA, 50 bp, synthetic construct
  source 1..50: mol_type = other DNA
ggattcataaggccctggagcagcggaaggagtacgaggttaggcttccg

SEQ ID NO: 405 - DNA, 50 bp, synthetic construct
  source 1..50: mol_type = other DNA
gggccagtgtcgggacagttgtcagcagggctccaacatgacgctcatcg

SEQ ID NO: 406 - DNA, 50 bp, synthetic construct
  source 1..50: mol_type = other DNA
cgggaaggccaagtcctttgctgtgagctctgtccccgggtttatcacgc

SEQ ID NO: 407 - DNA, 50 bp, synthetic construct
  source 1..50: mol_type = other DNA
cggaggggaaggtcaccaaaatgcttgtttatagaagcaacagcaactgg

SEQ ID NO: 408 - DNA, 50 bp, synthetic construct
  source 1..50: mol_type = other DNA
cggaagctcttttgtgcaagtaactagctgtattaaatgcaataattaca

SEQ ID NO: 409 - DNA, 50 bp, synthetic construct
  source 1..50: mol_type = other DNA
cgggaaacgtcattttgatgtcacgtttccatagcaacagagcaactgca

SEQ ID NO: 410 - DNA, 50 bp, synthetic construct
  source 1..50: mol_type = other DNA
ggtgcgcaggcagggcaactacctgctgtgctcactgctgctgggcaacg

SEQ ID NO: 411 - DNA, 50 bp, synthetic construct
  source 1..50: mol_type = other DNA
tccatgatgaccagagttggctggccctgagataccacacccaggagacg

SEQ ID NO: 412 - DNA, 50 bp, synthetic construct
  source 1..50: mol_type = other DNA
aactttttaccatactgctaaattaacttgtcaaaaggcttcaaacttcg

SEQ ID NO: 413 - DNA, 50 bp, synthetic construct
  source 1..50: mol_type = other DNA
gggtccaccaccagctgcagggcagcccggaactgctcatgggatactcg

SEQ ID NO: 414 - DNA, 50 bp, synthetic construct
  source 1..50: mol_type = other DNA
aatgtagcttttatctcgtagctgggacaactatcttcagcattctgtcg

SEQ ID NO: 415 - DNA, 50 bp, synthetic construct
  source 1..50: mol_type = other DNA
gtaccctgtcattttgtttcagcaacttgtccttctgaatgcacagttcg

SEQ ID NO: 416 - DNA, 50 bp, synthetic construct
  source 1..50: mol_type = other DNA
ttatcttaatttattgatgaaaacttgttttacggtccagagcacagacg

SEQ ID NO: 417 - DNA, 50 bp, synthetic construct
  source 1..50: mol_type = other DNA
cggcgggttgataggtaagttttaatctgacaaggccaaaaagtctatta

SEQ ID NO: 418 - DNA, 50 bp, synthetic construct
  source 1..50: mol_type = other DNA
cggctaacagatggactaaagatgtgagatgcagaaagatttaccaatct

SEQ ID NO: 419 - DNA, 50 bp, synthetic construct
  source 1..50: mol_type = other DNA
cgggatggctaagctccgctagctaaaagttacttcccattacttttgct

SEQ ID NO: 420 - DNA, 50 bp, synthetic construct
  source 1..50: mol_type = other DNA
gacaacaaccacatcagctgcattgaagatggagccttccgagcgctgcg

SEQ ID NO: 421 - DNA, 50 bp, synthetic construct
  source 1..50: mol_type = other DNA
cgctgcctacatctgtccttgtggctccgatggggtcttccttgcagtct

SEQ ID NO: 422 - DNA, 50 bp, synthetic construct
  source 1..50: mol_type = other DNA
cgctgatgacattgtgcactcgagatcttggtaatctttgggaaaattat

SEQ ID NO: 423 - DNA, 50 bp, synthetic construct
  source 1..50: mol_type = other DNA
cgaccttgaagatggtgagtagccagccaaagtacgatctaatacgggag

SEQ ID NO: 424 - DNA, 50 bp, synthetic construct
  source 1..50: mol_type = other DNA
cggcctacacgcgctaccagaccctggagctggagaaggagttccacttc

SEQ ID NO: 425 - DNA, 50 bp, synthetic construct
  source 1..50: mol_type = other DNA
cggaactatgatctccataattatgcaactggtagtccgggccatttgga

SEQ ID NO: 426 - DNA, 50 bp, synthetic construct
  source 1..50: mol_type = other DNA
ccaccactcaccttggaatttccgggagctgagcagtttaggtcagagcg

SEQ ID NO: 427 - DNA, 50 bp, synthetic construct
  source 1..50: mol_type = other DNA
cggttctattaggcaaggccgcagacttcctaggtactgtctggggtctg

SEQ ID NO: 428 - DNA, 50 bp, synthetic construct
  source 1..50: mol_type = other DNA
aggccttatttattacctctcccagagaaagcagactggggcaataatcg

SEQ ID NO: 429 - DNA, 50 bp, synthetic construct
  source 1..50: mol_type = other DNA
cgcacaggggccgtagccccactgtgtgtgggagccatgaggatcctggc

SEQ ID NO: 430 - DNA, 50 bp, synthetic construct
  source 1..50: mol_type = other DNA
cggcaatgtttatgatctcattgcaaaccgcttcctgtgaggtggccccg

SEQ ID NO: 431 - DNA, 50 bp, synthetic construct
  source 1..50: mol_type = other DNA
cgtcggttttcatacatgctcctggactgggcccagattttaaaaggatc

SEQ ID NO: 432 - DNA, 50 bp, synthetic construct
  source 1..50: mol_type = other DNA
cggtgagagtgaccagagtgttaggtcagtccaagggtcacttataggtc

SEQ ID NO: 433 - DNA, 50 bp, synthetic construct
  source 1..50: mol_type = other DNA
cgtacaactgcaatcataggtcatgcatgaattattaacctcggggagtt

SEQ ID NO: 434 - DNA, 50 bp, synthetic construct
  source 1..50: mol_type = other DNA
cgctgtctgtctgttattagggagataacagctcttataaatgtttcagt

SEQ ID NO: 435 - DNA, 50 bp, synthetic construct
  source 1..50: mol_type = other DNA
agctgagcagctgccatttcaatagaattaaagcttccgaatgataaacg

SEQ ID NO: 436 - DNA, 50 bp, synthetic construct
  source 1..50: mol_type = other DNA
cgcctttacaatagtttatgttgagagtggaatataaattgcattattaa

SEQ ID NO: 437 - DNA, 50 bp, synthetic construct
  source 1..50: mol_type = other DNA
cgtttcaatgcagtaaatattgattcagttgggggttttcaatttaaacc

SEQ ID NO: 438 - DNA, 50 bp, synthetic construct
  source 1..50: mol_type = other DNA
atccttaaaaagactaaaatagtttcatcctttcataacctgtcatcacg

SEQ ID NO: 439 - DNA, 50 bp, synthetic construct
  source 1..50: mol_type = other DNA
cgctgctattctgtcagcttaaagagtattttatgcttgatcaacagctc

SEQ ID NO: 440 - DNA, 50 bp, synthetic construct
  source 1..50: mol_type = other DNA
gaacaaaacgctttgaaatgcctcctctccatcactgccaacattgtccg

SEQ ID NO: 441 - DNA, 50 bp, synthetic construct
  source 1..50: mol_type = other DNA
cctctgcttagaaaaagctgtggataagccaccttccccttcaccaatcg

SEQ ID NO: 442 - DNA, 50 bp, synthetic construct
  source 1..50: mol_type = other DNA
agctcataaatcgtgaaagatcaccacttcttttcaaaagcctctccacg

SEQ ID NO: 443 - DNA, 50 bp, synthetic construct
  source 1..50: mol_type = other DNA
cggctcactggctataaaacgtcacgtggggccattaaagtaagttttat

SEQ ID NO: 444 - DNA, 50 bp, synthetic construct
  source 1..50: mol_type = other DNA
cgggaggaaaaaggactttgaaacttgaaaggaaagagcttgctttcaac

SEQ ID NO: 445 - DNA, 50 bp, synthetic construct
  source 1..50: mol_type = other DNA
atcagctattagtgcctaatcatatggtctatcgatcatgagattaatcg

SEQ ID NO: 446 - DNA, 50 bp, synthetic construct
  source 1..50: mol_type = other DNA
cgcaacagtctgtggaggaggaagagtctgaaatggcaactggaccaaag

SEQ ID NO: 447 - DNA, 50 bp, synthetic construct
  source 1..50: mol_type = other DNA
cgggctctttcatgccccatcagaattgtaaatgaggggtctgtgggcag

SEQ ID NO: 448 - DNA, 50 bp, synthetic construct
  source 1..50: mol_type = other DNA
tcaccatcctggacacactcgtttgtcaatgtccctctgaaaatgtgacg

SEQ ID NO: 449 - DNA, 50 bp, synthetic construct
  source 1..50: mol_type = other DNA
cgagtggacatccagaaaaagaggttttacctagacgtgaagcaaagctc

SEQ ID NO: 450 - DNA, 50 bp, synthetic construct
  source 1..50: mol_type = other DNA
cggccaatcccctccgaggcactgctcacgttacaatggccttgaccaca

SEQ ID NO: 451 - DNA, 50 bp, synthetic construct
  source 1..50: mol_type = other DNA
aatgtgcctacccatcgctgttgctccttgcagcctgcgggtcctcttcg

SEQ ID NO: 452 - DNA, 50 bp, synthetic construct
  source 1..50: mol_type = other DNA
cgcttcagaatatattggattggggtttgatggctatttcgtgttttgca

SEQ ID NO: 453 - DNA, 50 bp, synthetic construct
  source 1..50: mol_type = other DNA
cgcccagggttcaagtctccaaggagttgctggggcctctatttagcctg

SEQ ID NO: 454 - DNA, 50 bp, synthetic construct
  source 1..50: mol_type = other DNA
ctgtgctctggaccgtaaaacaagttttcatcaataaattaagataagcg

SEQ ID NO: 455 - DNA, 50 bp, synthetic construct
  source 1..50: mol_type = other DNA
tggaaaattttctgtaaccaggttaatagtaaccacagttttaacaaccg

SEQ ID NO: 456 - DNA, 50 bp, synthetic construct
  source 1..50: mol_type = other DNA
cgagaaaaaagtgagtgtgttcttgccctaacattctggatctgcttgca

SEQ ID NO: 457 - DNA, 50 bp, synthetic construct
  source 1..50: mol_type = other DNA
agccatcggaggtgactgacagatatgatttgggacaggtcatcaagacg

SEQ ID NO: 458 - DNA, 50 bp, synthetic construct
  source 1..50: mol_type = other DNA
cgggcccacaggcaggctggccttttatgcttgccgacgcagatggtccc

SEQ ID NO: 459 - DNA, 50 bp, synthetic construct
  source 1..50: mol_type = other DNA
cacccaggctttggggagacgctcattacttcttttaactgtgattaacg

SEQ ID NO: 460 - DNA, 50 bp, synthetic construct
  source 1..50: mol_type = other DNA
cgcaagggagcttctaccccattgcagttgcacaaaagaaattgaacact

SEQ ID NO: 461 - DNA, 50 bp, synthetic construct
  source 1..50: mol_type = other DNA
cgggatgtcttttgatttgcttctgttatttgcatgtctctcttgtgatg

SEQ ID NO: 462 - DNA, 50 bp, synthetic construct
  source 1..50: mol_type = other DNA
cgggtgtagttcccagttctggctcttttcaaggaggaggagcagaaaga

SEQ ID NO: 463 - DNA, 50 bp, synthetic construct
  source 1..50: mol_type = other DNA
cccaactttgaaataaccaatatgagtatttcagagcacgagggttaccg

SEQ ID NO: 464 - DNA, 50 bp, synthetic construct
  source 1..50: mol_type = other DNA
cgggttaggaggcaagcacattacagctccattttacctctgcaggcatc

SEQ ID NO: 465 - DNA, 50 bp, synthetic construct
  source 1..50: mol_type = other DNA
cggaataatacaggctaatgaaatacagcacagctttccattactgttag

SEQ ID NO: 466 - DNA, 50 bp, synthetic construct
  source 1..50: mol_type = other DNA
cgtgatgagagactaattcgttacgctgaaacattaacggcaatttaagg

SEQ ID NO: 467 - DNA, 50 bp, synthetic construct
  source 1..50: mol_type = other DNA
cgtgagaataatgaatgtggaaatacaactaaaaacatggcactcaaaac

SEQ ID NO: 468 - DNA, 50 bp, synthetic construct
  source 1..50: mol_type = other DNA
cgcttttaatcttgtctctagtggcgtgtgtaggggggaggactttattt

SEQ ID NO: 469 - DNA, 50 bp, synthetic construct
  source 1..50: mol_type = other DNA
cgcatgaagttgtcccacaccaaggtcagcgactggcaggttctctacaa

SEQ ID NO: 470 - DNA, 50 bp, synthetic construct
  source 1..50: mol_type = other DNA
gattgatgacagaataatttacgcttgtttctcagtttccagctggcacg

SEQ ID NO: 471 - DNA, 50 bp, synthetic construct
  source 1..50: mol_type = other DNA
gagaattgaattgaaaacaccttgacaaagaaagcaatttcttttcttcg

SEQ ID NO: 472 - DNA, 50 bp, synthetic construct
  source 1..50: mol_type = other DNA
gagatccatgccattgtagccgtagccgtacctgccggagtgcatgctcg

SEQ ID NO: 473 - DNA, 50 bp, synthetic construct
  source 1..50: mol_type = other DNA
cggcgattaaatgttgttttaaagagggagtgctcactgccctcccatct

SEQ ID NO: 474 - DNA, 50 bp, synthetic construct
  source 1..50: mol_type = other DNA
agtcgttaattaacggggcttgtcagctgggaagatgaattctgtcctcg

SEQ ID NO: 475 - DNA, 50 bp, synthetic construct
  source 1..50: mol_type = other DNA
cggtgctgggaatatttaaaatctatgggattaaaggcagattaaaaact

SEQ ID NO: 476 - DNA, 50 bp, synthetic construct
  source 1..50: mol_type = other DNA
acagggaggccatcacattaacttttacagcacttggggagccgtaaccg

SEQ ID NO: 477 - DNA, 50 bp, synthetic construct
  source 1..50: mol_type = other DNA
taaacaaaacagaaggttgtaactgtcataatttgcattgcactttcacg

SEQ ID NO: 478 - DNA, 50 bp, synthetic construct
  source 1..50: mol_type = other DNA
cgcccagaaatttccctgagctttatcatgattcctggaggcagtctgat

SEQ ID NO: 479 - DNA, 50 bp, synthetic construct
  source 1..50: mol_type = other DNA
cggcccggctggtgtcccacaggctcggcgcagtaagttcagattattgc

SEQ ID NO: 480 - DNA, 50 bp, synthetic construct
  source 1..50: mol_type = other DNA
caatgcagtaaatattgattcagttgggggttttcaatttaaaccactcg

SEQ ID NO: 481 - DNA, 50 bp, synthetic construct
  source 1..50: mol_type = other DNA
gctgctgcggtaagcacatcgaccagagctgatcattacaagggcgtacg

SEQ ID NO: 482 - DNA, 50 bp, synthetic construct
  source 1..50: mol_type = other DNA
tcacgggattatattgttcgttgagtagcagctttctgtcccagctgtcg

SEQ ID NO: 483 - DNA, 50 bp, synthetic construct
  source 1..50: mol_type = other DNA
gctcctggaatggatcacaagagactcattagaaaaaacaaatattatcg

SEQ ID NO: 484 - DNA, 50 bp, synthetic construct
  source 1..50: mol_type = other DNA
cgggcagccaggaccgaataagcacaaaaatccctctagagtgttaaata

SEQ ID NO: 485 - DNA, 50 bp, synthetic construct
  source 1..50: mol_type = other DNA
ctcacctccagcggggcagataaagttactgttggcgagctgaggctacg

SEQ ID NO: 486 - DNA, 50 bp, synthetic construct
  source 1..50: mol_type = other DNA
gaaaggatgggcagactgggactcccgttgctatagcaacccagcttccg

SEQ ID NO: 487 - DNA, 50 bp, synthetic construct
  source 1..50: mol_type = other DNA
cgtcgtgcatgtcctgggactttttgacaggaggtgtgagcttagcagct

SEQ ID NO: 488 - DNA, 50 bp, synthetic construct
  source 1..50: mol_type = other DNA
ctgaatgattaaaatacactaattaaaattttgtgttccttggtaactcg

SEQ ID NO: 489 - DNA, 50 bp, synthetic construct
  source 1..50: mol_type = other DNA
cggagggtagggaaacgagacagccaccacgcgagatgaaataaacatga

SEQ ID NO: 490 - DNA, 50 bp, synthetic construct
  source 1..50: mol_type = other DNA
cttattcctgttccctttaccctttgacaacttgaaatgttaccacttcg

SEQ ID NO: 491 - DNA, 50 bp, synthetic construct
  source 1..50: mol_type = other DNA
actcgacaggcgtttgtcggacaaaatcaacactaatcaacatttattcg

SEQ ID NO: 492 - DNA, 50 bp, synthetic construct
  source 1..50: mol_type = other DNA
cgagttactgcaggagtgaactgtaaattccatgaaaccatgtaatatat

SEQ ID NO: 493 - DNA, 50 bp, synthetic construct
  source 1..50: mol_type = other DNA
tcatagttccgtgagcgagcaattcagggactcggcgagcatgcactccg

SEQ ID NO: 494 - DNA, 50 bp, synthetic construct
  source 1..50: mol_type = other DNA
cggcaatgacccacaacagggagacatcaagcgtgaaatttactagaaat

SEQ ID NO: 495 - DNA, 50 bp, synthetic construct
  source 1..50: mol_type = other DNA
agtctgcggccaaccgagaattgtggctggacatctgtggctgagctccg

SEQ ID NO: 496 - DNA, 50 bp, synthetic construct
  source 1..50: mol_type = other DNA
cggaaggctccatcttcaatgcagctgatgtggttgttgtccagttgcct

SEQ ID NO: 497 - DNA, 50 bp, synthetic construct
  source 1..50: mol_type = other DNA
gcactccggcaggtacggctacggctacaatggcatggatctcagcgtcg

SEQ ID NO: 498 - DNA, 50 bp, synthetic construct
  source 1..50: mol_type = other DNA
cgatgaaagattattttaaaatggcactgatagtgtttagacattgtcat

SEQ ID NO: 499 - DNA, 50 bp, synthetic construct
  source 1..50: mol_type = other DNA
tttttatccaaatggctcaattaccgcaaagccccacagagaaaattacg

SEQ ID NO: 500 - DNA, 50 bp, synthetic construct
  source 1..50: mol_type = other DNA
catatcaaaaaacaaatgagctcttattttgtaaactcattttgcggtcg

SEQ ID NO: 501 - DNA, 50 bp, synthetic construct
  source 1..50: mol_type = other DNA
cggcttgcttgcataaataaattaaaagatgcaaaactaaagtttaaaca

SEQ ID NO: 502 - DNA, 50 bp, synthetic construct
  source 1..50: mol_type = other DNA
cgctttggtcaccacaggtgtctctgtaacctgcaaactcttattactag

SEQ ID NO: 503 - DNA, 50 bp, synthetic construct
  source 1..50: mol_type = other DNA
cgtttgcctgtctcagtggattgaacctcaaagactcctttgacgcaagt

SEQ ID NO: 504 - DNA, 50 bp, synthetic construct
  source 1..50: mol_type = other DNA
attcatctggttcattagtgcatctcgatgctgcccctgacagctgctcg

SEQ ID NO: 505 - DNA, 50 bp, synthetic construct
  source 1..50: mol_type = other DNA
gcccgctgctcctgggagccggtgctgctgccactcagagagttccttcg

SEQ ID NO: 506 - DNA, 50 bp, synthetic construct
  source 1..50: mol_type = other DNA
tagcatctcaacagaacgccaccgtgctgaagttcggagagcagtaaacg

SEQ ID NO: 507 - DNA, 50 bp, synthetic construct
  source 1..50: mol_type = other DNA
actgaaaccctaaccttcgggcttccccactaatttgtgataaggcctcg

SEQ ID NO: 508 - DNA, 50 bp, synthetic construct
  source 1..50: mol_type = other DNA
tttgtagtgggtcactgattctgctggggtattatcttctttactactcg

SEQ ID NO: 509 - DNA, 50 bp, synthetic construct
  source 1..50: mol_type = other DNA
agaactagaagatgctgccagatggatatggaccacttcagtgaatctcg

SEQ ID NO: 510 - DNA, 50 bp, synthetic construct
  source 1..50: mol_type = other DNA
aaaaaaactccaattggcttttcatacatccaccctccccaaaaataacg

SEQ ID NO: 511 - DNA, 50 bp, synthetic construct
  source 1..50: mol_type = other DNA
gtggactaagctcttgggggctctcgtttgccagccaaattgattttccg

SEQ ID NO: 512 - DNA, 50 bp, synthetic construct
  source 1..50: mol_type = other DNA
tatttggaagctttgatgagagcctgctggcacgcgccgaagctctggcg

SEQ ID NO: 513 - DNA, 50 bp, synthetic construct
  source 1..50: mol_type = other DNA
atctcgacccttgctgtgctgctgatgcagctcaaggtcacactaacccg

SEQ ID NO: 514 - DNA, 50 bp, synthetic construct
  source 1..50: mol_type = other DNA
tgcctttgaattatttcacaggcccgctgctttacatttacactctgtcg

SEQ ID NO: 515 - DNA, 50 bp, synthetic construct
  source 1..50: mol_type = other DNA
cggagatgtaatttaaacacatctgttcaaatatgcatttcagatcaact

SEQ ID NO: 516 - DNA, 50 bp, synthetic construct
  source 1..50: mol_type = other DNA
cctgtttaatcatctcattttctggtggccgtttattcctcgtttccacg

SEQ ID NO: 517 - DNA, 50 bp, synthetic construct
  source 1..50: mol_type = other DNA
cggcaaagtgtccattatcagcaggtcgggagcagtaaattggagcagtt

SEQ ID NO: 518 - DNA, 50 bp, synthetic construct
  source 1..50: mol_type = other DNA
aaagtttaaaaacaaaaacccttaacggaactgccttaaaaaggcagacg

SEQ ID NO: 519 - DNA, 50 bp, synthetic construct
  source 1..50: mol_type = other DNA
cgctcctggggtaattaggaccatgtgttcttcctaaaacagggctgggg

SEQ ID NO: 520 - DNA, 50 bp, synthetic construct
  source 1..50: mol_type = other DNA
cggactgtagattgctctgccattctgagttcagcaatttgaataaatta

SEQ ID NO: 521 - DNA, 50 bp, synthetic construct
  source 1..50: mol_type = other DNA
cgcaattagccctctgtgaaaggggcccctcacagccccctcggccaatc

SEQ ID NO: 522 - DNA, 50 bp, synthetic construct
  source 1..50: mol_type = other DNA
cggccccagaaagtgcagattcaggttttaatacacaaaattatttcagg

SEQ ID NO: 523 - DNA, 50 bp, synthetic construct
  source 1..50: mol_type = other DNA
cgccttcccagcagcagtgaggattctgacgacaatggcagtaagtcctg

SEQ ID NO: 524 - DNA, 50 bp, synthetic construct
  source 1..50: mol_type = other DNA
cgctgagatccatgccattgtagccgtagccgtacctgccggagtgcatg

SEQ ID NO: 525 - DNA, 50 bp, synthetic construct
  source 1..50: mol_type = other DNA
cggaagctttaattctattgaaatggcagctgctcagctccggggcacag

SEQ ID NO: 526 - DNA, 50 bp, synthetic construct
  source 1..50: mol_type = other DNA
aatggcagggaaagcttccgacggctccatcaaatggcagctctgctacg

SEQ ID NO: 527 - DNA, 50 bp, synthetic construct
  source 1..50: mol_type = other DNA
atcattgttgaaatattccctttgaactgccgtgtccacgggggtctccg

SEQ ID NO: 528 - DNA, 50 bp, synthetic construct
  source 1..50: mol_type = other DNA
cgatgcaaattgccataaagtgggataatcaggcagtagaagtgtcataa

SEQ ID NO: 529 - DNA, 50 bp, synthetic construct
  source 1..50: mol_type = other DNA
cgagatctgtttgaccaatacatttatatttatgatgactgagggcagat

SEQ ID NO: 530 - DNA, 50 bp, synthetic construct
  source 1..50: mol_type = other DNA
agagctgcgggcaactcatgcatgttttatcctccatctggaccggcacg

SEQ ID NO: 531 - DNA, 50 bp, synthetic construct
  source 1..50: mol_type = other DNA
agggaagcagcccattctgacagactgagcaataatctgaacttactgcg

SEQ ID NO: 532 - DNA, 50 bp, synthetic construct
  source 1..50: mol_type = other DNA
gataatggcctgctaaacagagcttctgccgctgccacatctgtatttcg

SEQ ID NO: 533 - DNA, 50 bp, synthetic construct
  source 1..50: mol_type = other DNA
ctttctggggccgaaagcctcgcttaattatggcgggtgtccttgggacg

SEQ ID NO: 534 - DNA, 50 bp, synthetic construct
  source 1..50: mol_type = other DNA
cgcctactcttgttggatccggttataaagtaaactatgttctttgagaa

SEQ ID NO: 535 - DNA, 50 bp, synthetic construct
  source 1..50: mol_type = other DNA
cggaagataagggttttgaaagtttctattgtgattatagcctgtaactc

SEQ ID NO: 536 - DNA, 50 bp, synthetic construct
  source 1..50: mol_type = other DNA
atagagctttcactgagcttgtgctatgacctaagccttcacgcagaacg

SEQ ID NO: 537 - DNA, 50 bp, synthetic construct
  source 1..50: mol_type = other DNA
cgagtttgcacttctgtttacaaagcaatcctgccatcaaaagaggaaca

SEQ ID NO: 538 - DNA, 50 bp, synthetic construct
  source 1..50: mol_type = other DNA
cgctttcaggaattcttttgcctgaggtaatgggaagctaaggttgcttt

SEQ ID NO: 539 - DNA, 50 bp, synthetic construct
  source 1..50: mol_type = other DNA
cggcttctgctgtgttgccttgaaatttattattctggagatttaaagaa

SEQ ID NO: 540 - DNA, 50 bp, synthetic construct
  source 1..50: mol_type = other DNA
atgttttacacagctgttccttggtcaaggtcatttgcaagagcaagtcg

SEQ ID NO: 541 - DNA, 50 bp, synthetic construct
  source 1..50: mol_type = other DNA
tgcataattatggagatcatagttccgtgagcgagcaattcagggactcg

SEQ ID NO: 542 - DNA, 50 bp, synthetic construct
  source 1..50: mol_type = other DNA
cgaggaggctgaacactgcatgaagagaggaccgagccaatgagagaaca

SEQ ID NO: 543 - DNA, 50 bp, synthetic construct
  source 1..50: mol_type = other DNA
aataaatctgcagtctgtcttcctggtaatccatcacagattgcaaatcg

SEQ ID NO: 544 - DNA, 50 bp, synthetic construct
  source 1..50: mol_type = other DNA
cggcatctcctaatcagctaccatgtgaaatgcaaattcagacagtctga

SEQ ID NO: 545 - DNA, 50 bp, synthetic construct
  source 1..50: mol_type = other DNA
aaataggatgcatacggctccaatatagctcatatggtcgcattcattcg

SEQ ID NO: 546 - DNA, 50 bp, synthetic construct
  source 1..50: mol_type = other DNA
cgcgccattcctgatgacaaactgccagccagctcccacaaaagcacgat

SEQ ID NO: 547 - DNA, 50 bp, synthetic construct
  source 1..50: mol_type = other DNA
cggggagcttggaatgcattcagttccatcaggggatttgtgtccctcgt

SEQ ID NO: 548 - DNA, 50 bp, synthetic construct
  source 1..50: mol_type = other DNA
cgctaagtgtctgagactgacatcggaaccagagggggactggttttgtc

SEQ ID NO: 549 - DNA, 50 bp, synthetic construct
  source 1..50: mol_type = other DNA
tcaaccctcttattcattttgatttatttctatccgtaagcagactaacg

SEQ ID NO: 550 - DNA, 50 bp, synthetic construct
  source 1..50: mol_type = other DNA
ttaaatgcatatttgcgacttccaacattcttcattgtattttctcttcg

SEQ ID NO: 551 - DNA, 50 bp, synthetic construct
  source 1..50: mol_type = other DNA
aaggtgacagccaagccgcgttagcacctgcataaaatattaaagggacg

SEQ ID NO: 552 - DNA, 50 bp, synthetic construct
  source 1..50: mol_type = other DNA
cgtgccacaaatggtattttactctcaataagaactcgagctgagtttat

SEQ ID NO: 553 - DNA, 50 bp, synthetic construct
  source 1..50: mol_type = other DNA
cgtgagcgagcaattcagggactcggcgagcatgcactccggcaggtacg

SEQ ID NO: 554 - DNA, 50 bp, synthetic construct
  source 1..50: mol_type = other DNA
cccgctctctgctgcgtataaaattacttctgagtcggctggactgaacg

SEQ ID NO: 555 - DNA, 50 bp, synthetic construct
  source 1..50: mol_type = other DNA
attttgtgtattaaaacctgaatctgcactttctggggccgaaagcctcg

SEQ ID NO: 556 - DNA, 50 bp, synthetic construct
  source 1..50: mol_type = other DNA
acgttttatagccagtgagccgatctgtctgtgctatggatgattttacg

SEQ ID NO: 557 - DNA, 50 bp, synthetic construct
  source 1..50: mol_type = other DNA
cgggccctggcatttgcccggaacgtcagcctgtaggtctcaatctgcag

SEQ ID NO: 558 - DNA, 50 bp, synthetic construct
  source 1..50: mol_type = other DNA
tggaactgagggcaccagctcagcaggtagtcccagttatagctgccacg

SEQ ID NO: 559 - DNA, 50 bp, synthetic construct
  source 1..50: mol_type = other DNA
atgcgaaggaactctctgagtggcagcagcaccggctcccaggagcagcg

SEQ ID NO: 560 - DNA, 50 bp, synthetic construct
  source 1..50: mol_type = other DNA
cgcttgcgattagctgctggtagtgccataatgaagcttgctcaggaacc

SEQ ID NO: 561 - DNA, 50 bp, synthetic construct
  source 1..50: mol_type = other DNA
tatacaagccatgattcatgacagaattttacattccatgagagattacg

SEQ ID NO: 562 - DNA, 50 bp, synthetic construct
  source 1..50: mol_type = other DNA
cggacagatggcggggacgatgttatttatatcaatcagccaaaatcctc

SEQ ID NO: 563 - DNA, 50 bp, synthetic construct
  source 1..50: mol_type = other DNA
cggtcagagtgatttggggctggttagagatgatttaaaaggaaggttat

SEQ ID NO: 564 - DNA, 50 bp, synthetic construct
  source 1..50: mol_type = other DNA
cgatcaaggtatggggcatttcctggggattaatgaccagctgggggagt

SEQ ID NO: 565 - DNA, 50 bp, synthetic construct
  source 1..50: mol_type = other DNA
tgatcctttcataaagagagtttctggtgtcaggagacagccgcccagcg

SEQ ID NO: 566 - DNA, 50 bp, synthetic construct
  source 1..50: mol_type = other DNA
tttgttgatttattgtacgttagccaccgagccgtgcaaagaacattacg

SEQ ID NO: 567 - DNA, 50 bp, synthetic construct
  source 1..50: mol_type = other DNA
cgctcacggaactatgatctccataattatgcaactggtagtccgggcca

SEQ ID NO: 568 - DNA, 50 bp, synthetic construct
  source 1..50: mol_type = other DNA
aattcttttttctggatcaaaggctggctgtgcataattgaatggtaacg

SEQ ID NO: 569 - DNA, 50 bp, synthetic construct
  source 1..50: mol_type = other DNA
gcatagtttttagcataacatcttgactcctcgataattatatccgttcg

SEQ ID NO: 570 - DNA, 50 bp, synthetic construct
  source 1..50: mol_type = other DNA
agcagtgccctggcttaacaagcagttttataaatctgaagctgtctccg

SEQ ID NO: 571 - DNA, 50 bp, synthetic construct
  source 1..50: mol_type = other DNA
taatggtagacagactgcttaggagacgccagtctgtgatttagctgtcg

SEQ ID NO: 572 - DNA, 50 bp, synthetic construct
  source 1..50: mol_type = other DNA
cggtcactctggtggtataaatatgtttatagcggaccaaggggaaatga

SEQ ID NO: 573 - DNA, 50 bp, synthetic construct
  source 1..50: mol_type = other DNA
aatttcatgccccaatgagaagagcaaggtcgaagcaaatgcttccatcg

SEQ ID NO: 574 - DNA, 50 bp, synthetic construct
  source 1..50: mol_type = other DNA
cgggctaatgaggtaaactgaagcgagccctgtcccccctgacaggttta

SEQ ID NO: 575 - DNA, 50 bp, synthetic construct
  source 1..50: mol_type = other DNA
cgacagggagagtagtaaaactgccccttaagatggcttttaataggaag

SEQ ID NO: 576 - DNA, 50 bp, synthetic construct
  source 1..50: mol_type = other DNA
cggcaccaagttgatctgaaatgcatatttgaacagatgtgtttaaatta

SEQ ID NO: 577 - DNA, 50 bp, synthetic construct
  source 1..50: mol_type = other DNA
cctctgcctaaaattgggcccgctgcaaggaaccctgggtcacttctacg

SEQ ID NO: 578 - DNA, 50 bp, synthetic construct
  source 1..50: mol_type = other DNA
cgaaggttttactgagagaacaacaggagctgtcttgattattgtctgca

SEQ ID NO: 579 - DNA, 50 bp, synthetic construct
  source 1..50: mol_type = other DNA
catgttacaggaagccacagctcgccccaggggtgctgcaaacagacacg

SEQ ID NO: 580 - DNA, 50 bp, synthetic construct
  source 1..50: mol_type = other DNA
cgggattctgtcactttggcaaccttcagagacaacatttttccagtgtt

SEQ ID NO: 581 - DNA, 50 bp, synthetic construct
  source 1..50: mol_type = other DNA
cggtgaactgtgaaatataaaaattgcataccaatacatttttatggaaa

SEQ ID NO: 582 - DNA, 50 bp, synthetic construct
  source 1..50: mol_type = other DNA
cgggaattctctagggcatgtggattagctcggaaagcctgaatgattaa

SEQ ID NO: 583 - DNA, 50 bp, synthetic construct
  source 1..50: mol_type = other DNA
cggaaataaaccctgtcagaaaatgagaagtgactccccctttggatggc

SEQ ID NO: 584 - DNA, 50 bp, synthetic construct
  source 1..50: mol_type = other DNA
cggttgctttctagcttgctgctcagctaaaaagaaaccactttggctga

SEQ ID NO: 585 - DNA, 50 bp, synthetic construct
  source 1..50: mol_type = other DNA
cggggtccttaatgaccctggcaggccaatgattttgttttgtttgagag

SEQ ID NO: 586 - DNA, 50 bp, synthetic construct
  source 1..50: mol_type = other DNA
gggtggcatttccttcagtgttaacaacagaggttaactgcacacagacg

SEQ ID NO: 587 - DNA, 50 bp, synthetic construct
  source 1..50: mol_type = other DNA
cggccaataagaattatttgccttattaaagcagcagaagacgctgatta

SEQ ID NO: 588 - DNA, 50 bp, synthetic construct
  source 1..50: mol_type = other DNA
aatagcacagaagtcagaggcaagtttgaaagtacaccagaaataaaacg

SEQ ID NO: 589 - DNA, 50 bp, synthetic construct
  source 1..50: mol_type = other DNA
tgcacagctaagcatcttcttgattacaatccgccacacttctaactgcg

SEQ ID NO: 590 - DNA, 50 bp, synthetic construct
  source 1..50: mol_type = other DNA
tttgaaacagccattgaaaacaaattacatctgcactgccttttcatccg

SEQ ID NO: 591 - DNA, 50 bp, synthetic construct
  source 1..50: mol_type = other DNA
agtggtaaccatccaccacttgagtcatctcgggtcatcaccacagctcg

SEQ ID NO: 592 - DNA, 50 bp, synthetic construct
  source 1..50: mol_type = other DNA
cacagccgagcctcttgaagccattcttacagatgatgaaccagaccacg

SEQ ID NO: 593 - DNA, 50 bp, synthetic construct
  source 1..50: mol_type = other DNA
cggagagcagtaaacgatgaacggttaacaacaattgcacataagtaagc

SEQ ID NO: 594 - RNA, 50 nt, synthetic construct
  source 1..50: mol_type = other RNA
ttaattcaactcctgctgttaaattaaccagcgttccagccgagttaacg

SEQ ID NO: 595 - DNA, 50 bp, synthetic construct
  source 1..50: mol_type = other DNA
atactgcgaggcagaaacatgtggacacagctggagtgtcatgtgtcacg

SEQ ID NO: 596 - DNA, 50 bp, synthetic construct
  source 1..50: mol_type = other DNA
ggctgcagggcatgctgggagctggcctcattctgggccaccggccaccg

SEQ ID NO: 597 - DNA, 50 bp, synthetic construct
  source 1..50: mol_type = other DNA
cagttcctcacatttgagtactactcagattggcagccttcacttttccg

SEQ ID NO: 598 - DNA, 50 bp, synthetic construct
  source 1..50: mol_type = other DNA
ggaacggccccataatgtaactgattaatgaaggattgctgaggcatacg

SEQ ID NO: 599 - DNA, 50 bp, synthetic construct
  source 1..50: mol_type = other DNA
ccagcagttacccattgatcatagaaaatgcccagttcatcagtttaacg

SEQ ID NO: 600 - DNA, 50 bp, synthetic construct
  source 1..50: mol_type = other DNA
ggagttgacatccaacattatataccacataacatataatctcactaacg

SEQ ID NO: 601 - DNA, 50 bp, synthetic construct
  source 1..50: mol_type = other DNA
cgccagtctgtgatttagctgtcgccagacaccccatgttttttgtgaga

SEQ ID NO: 602 - DNA, 50 bp, synthetic construct
  source 1..50: mol_type = other DNA
gtgcttcataacaccccatgtgtgtgtttatctcatgataaccacactcg

SEQ ID NO: 603 - DNA, 50 bp, synthetic construct
  source 1..50: mol_type = other DNA
cggcctttgatcaggacacagagcccacgtccgagccttttattactgtc

SEQ ID NO: 604 - DNA, 50 bp, synthetic construct
  source 1..50: mol_type = other DNA
aattgtgctgaagaatgactacgacaccctgttgagatcagatccaagcg

SEQ ID NO: 605 - DNA, 50 bp, synthetic construct
  source 1..50: mol_type = other DNA
cggcttaataggaacagctggtcagtgaagtgtttgtgagcaatttaaca

SEQ ID NO: 606 - DNA, 50 bp, synthetic construct
  source 1..50: mol_type = other DNA
gcatgaagctgagatggaaacccagtgcattaatttacctctcatcttcg

SEQ ID NO: 607 - DNA, 50 bp, synthetic construct
  source 1..50: mol_type = other DNA
ttatcaggaacataactgccactgccgagatcaaaacactgcacagcacg

SEQ ID NO: 608 - DNA, 50 bp, synthetic construct
  source 1..50: mol_type = other DNA
tcgcagttccattaggatgtaccaattgttaggccgtcagctgccgatcg

SEQ ID NO: 609 - DNA, 50 bp, synthetic construct
  source 1..50: mol_type = other DNA
cgagtacaggtgatgaaatcttggcagtgttaacaaaaaagtagtgtgta

SEQ ID NO: 610 - DNA, 50 bp, synthetic construct
  source 1..50: mol_type = other DNA
acttcatagacctgagaggcggtttgagaggtcagcccttttcaggttcg

SEQ ID NO: 611 - DNA, 50 bp, synthetic construct
  source 1..50: mol_type = other DNA
cgcattgacacgatagaccggaacagctcagaagaagctgttatggtcta

SEQ ID NO: 612 - DNA, 50 bp, synthetic construct
  source 1..50: mol_type = other DNA
tcccaccttggggatgggcagggacaggtcacaaaaaacctcgaaggtcg

SEQ ID NO: 613 - DNA, 50 bp, synthetic construct
  source 1..50: mol_type = other DNA
cagataacgactggctattcattatcttcgccgggaacaaagattagccg

SEQ ID NO: 614 - DNA, 50 bp, synthetic construct
  source 1..50: mol_type = other DNA
ggagccgttctgaatctgctggtccatgctgttcaggagccccaggttcg

SEQ ID NO: 615 - DNA, 50 bp, synthetic construct
  source 1..50: mol_type = other DNA
gattgatcaccccagactttgttcatgaatatttaaatatcgctgattcg

SEQ ID NO: 616 - DNA, 50 bp, synthetic construct
  source 1..50: mol_type = other DNA
taatgagaactcacgaatcctgtcatcggctcctcctggaaatccaggcg

SEQ ID NO: 617 - DNA, 50 bp, synthetic construct
  source 1..50: mol_type = other DNA
tgcgtcagctctgccttataaatggagtgcattctatcaaaaccaggacg

SEQ ID NO: 618 - DNA, 50 bp, synthetic construct
  source 1..50: mol_type = other DNA
gtggtacgcagaagtcaaccgggcagagtctttcagacagctgtgggacg

SEQ ID NO: 619 - DNA, 50 bp, synthetic construct
  source 1..50: mol_type = other DNA
ccaactagatgactgggtattcctgcaagcaggcccctttccagccctcg

SEQ ID NO: 620 - DNA, 50 bp, synthetic construct
  source 1..50: mol_type = other DNA
gagtgtccccagaaggcccacaacaggaatctgagggaggctacttgacg

SEQ ID NO: 621 - DNA, 50 bp, synthetic construct
  source 1..50: mol_type = other DNA
cgggcccaattttaggcagagggtttaaatagcttatttattattttgta

SEQ ID NO: 622 - DNA, 50 bp, synthetic construct
  source 1..50: mol_type = other DNA
cggccggcgcagcttagagaaatcaaaacaactggttaaaccccagccca

SEQ ID NO: 623 - DNA, 50 bp, synthetic construct
  source 1..50: mol_type = other DNA
cgagcactgaggcgacttgtagagccgctccccatcagtactttccaggg

SEQ ID NO: 624 - DNA, 50 bp, synthetic construct
  source 1..50: mol_type = other DNA
cgcaggttgatcagcgaaacagcatccattttaatctgcggggagcccct

SEQ ID NO: 625 - DNA, 50 bp, synthetic construct
  source 1..50: mol_type = other DNA
cgtgagttgaaatagcagaaagtggatttgtaacttaaaaagtgtaaaca

SEQ ID NO: 626 - DNA, 50 bp, synthetic construct
  source 1..50: mol_type = other DNA
cgccaggctgacaaggtgtggcggctggacctggtcatggtgattttgtt

SEQ ID NO: 627 - DNA, 50 bp, synthetic construct
  source 1..50: mol_type = other DNA
atctgctggcacacaccggcaatctggcactcatctgtgtccacacagcg

SEQ ID NO: 628 - DNA, 50 bp, synthetic construct
  source 1..50: mol_type = other DNA
cggaggaatgaatggtttggggaaaatctcacacaggaataaatttacaa

SEQ ID NO: 629 - DNA, 50 bp, synthetic construct
  source 1..50: mol_type = other DNA
cgcctcagtggaatggaaacttcattttgagtctaatattagaccttgct

SEQ ID NO: 630 - DNA, 50 bp, synthetic construct
  source 1..50: mol_type = other DNA
cgcaattcttgtacgagtctgcggccaaccgagaattgtggctggacatc

SEQ ID NO: 631 - DNA, 50 bp, synthetic construct
  source 1..50: mol_type = other DNA
cgtggctttgaagaattataggaattttatgatgtgctgttggagttata

SEQ ID NO: 632 - DNA, 50 bp, synthetic construct
  source 1..50: mol_type = other DNA
aaatacccctcagaaacacacataaaaaagccaaatccaacaacagaacg

SEQ ID NO: 633 - DNA, 50 bp, synthetic construct
  source 1..50: mol_type = other DNA
gacagatgggagggcagtgagcactccctctttaaaacaacatttaatcg

SEQ ID NO: 634 - DNA, 50 bp, synthetic construct
  source 1..50: mol_type = other DNA
cggcttggcaaagtccgagtccttgctgtccaaagatggactccgatgga

SEQ ID NO: 635 - DNA, 50 bp, synthetic construct
  source 1..50: mol_type = other DNA
agagagaataactctaagtaggtcaggcctgtatttctaaaaatgctacg

SEQ ID NO: 636 - DNA, 50 bp, synthetic construct
  source 1..50: mol_type = other DNA
cggcaaagtacaaaatggtaattaaaaatttctagttcagtgcgaggcac

SEQ ID NO: 637 - DNA, 50 bp, synthetic construct
  source 1..50: mol_type = other DNA
cggatgggagcactcgacagaaaaatcaaacttctggtcatggctcatta

SEQ ID NO: 638 - DNA, 50 bp, synthetic construct
  source 1..50: mol_type = other DNA
tatgtcagaagttggaggtcagccatccaagtacatgaaaacttggctcg

SEQ ID NO: 639 - DNA, 50 bp, synthetic construct
  source 1..50: mol_type = other DNA
tcatccttgaacaccacgctgccttcaactagtgcctggtcatccattcg

SEQ ID NO: 640 - DNA, 50 bp, synthetic construct
  source 1..50: mol_type = other DNA
cgggaggaacctgccatgtccatggatgccaatggaaagataatttgggc

SEQ ID NO: 641 - DNA, 50 bp, synthetic construct
  source 1..50: mol_type = other DNA
cgggccatcggcgtgctgtgggccatcttcaccatctgcttcgccatcat

SEQ ID NO: 642 - DNA, 50 bp, synthetic construct
  source 1..50: mol_type = other DNA
cgtgggtttgactggcagctttcctcatctgtcgatcacagggtaataaa

SEQ ID NO: 643 - DNA, 50 bp, synthetic construct
  source 1..50: mol_type = other DNA
cgggcagttctcagacaatgactgcaggcattaatgccgacggacatttg

SEQ ID NO: 644 - DNA, 50 bp, synthetic construct
  source 1..50: mol_type = other DNA
cctgtgtgacacctgtttttgtctcctctgatcctacgacaggttttccg

SEQ ID NO: 645 - DNA, 50 bp, synthetic construct
  source 1..50: mol_type = other DNA
cgcccccgccccagattagatctccacgtggtgctttgcagtgatccttg

SEQ ID NO: 646 - DNA, 50 bp, synthetic construct
  source 1..50: mol_type = other DNA
cacacagcagatgtatccaaacacataaaaatctctacagtctggggtcg

SEQ ID NO: 647 - DNA, 50 bp, synthetic construct
  source 1..50: mol_type = other DNA
ccgtccttatttaattttattgctgaaattcatctttttagcctcctccg

SEQ ID NO: 648 - DNA, 50 bp, synthetic construct
  source 1..50: mol_type = other DNA
cgctctctgaaagggctccgataatctggaaggcagagataaggaacacc

SEQ ID NO: 649 - DNA, 50 bp, synthetic construct
  source 1..50: mol_type = other DNA
taccaccccatcctggtgtcccgtctgttcataaataactctttcatccg

SEQ ID NO: 650 - DNA, 50 bp, synthetic construct
  source 1..50: mol_type = other DNA
cggctgcaataaatcaataggaaaatagaagctgccagttagactaaaca

SEQ ID NO: 651 - DNA, 50 bp, synthetic construct
  source 1..50: mol_type = other DNA
actgagcaataatctgaacttactgcgccgagcctgtgggacaccagccg

SEQ ID NO: 652 - DNA, 50 bp, synthetic construct
  source 1..50: mol_type = other DNA
cgggtgtctgacagccggacagcgggatctgcacagcaagtgaaattccc

SEQ ID NO: 653 - DNA, 50 bp, synthetic construct
  source 1..50: mol_type = other DNA
cgccgctccctgctggaaatattgaatagtgctgctgacattctgatcaa

SEQ ID NO: 654 - DNA, 50 bp, synthetic construct
  source 1..50: mol_type = other DNA
tctactcaatggattgtggtcaaggccattgtaacgtgagcagtgcctcg

SEQ ID NO: 655 - DNA, 50 bp, synthetic construct
  source 1..50: mol_type = other DNA
cgggtaaattacagtcatttactgggagtgtaatttactgagagccttta

SEQ ID NO: 656 - DNA, 50 bp, synthetic construct
  source 1..50: mol_type = other DNA
ccccaacctgacggcttctctctttacacacagtgaagttcaagtcctcg

SEQ ID NO: 657 - DNA, 50 bp, synthetic construct
  source 1..50: mol_type = other DNA
cgtgagaagggaagataaattttgtcctagtaatactatagcgactcata

SEQ ID NO: 658 - DNA, 50 bp, synthetic construct
  source 1..50: mol_type = other DNA
cggctccaatatagctcatatggtcgcattcattcgatcacagcagtgct

SEQ ID NO: 659 - DNA, 50 bp, synthetic construct
  source 1..50: mol_type = other DNA
cgcaacataaaagcacgtaaattggttccagccatctctagtaaaaactt

SEQ ID NO: 660 - DNA, 50 bp, synthetic construct
  source 1..50: mol_type = other DNA
aattaacacccttaaatttgttgattactttcacgggattatattgttcg

SEQ ID NO: 661 - DNA, 50 bp, synthetic construct
  source 1..50: mol_type = other DNA
cgcccggggcatcatcatctttgccaatgaggatgacatcaggtgggaca

SEQ ID NO: 662 - DNA, 50 bp, synthetic construct
  source 1..50: mol_type = other DNA
cgcggtcgtttgtgcgtctatagcacccttgcacaatttatgatgaatta

SEQ ID NO: 663 - DNA, 50 bp, synthetic construct
  source 1..50: mol_type = other DNA
cgggctcccccggccttaatgagataatcaatacttgttctcggcttaat

SEQ ID NO: 664 - DNA, 50 bp, synthetic construct
  source 1..50: mol_type = other DNA
catcggcagattctttcattatacaagatttagcatctgctcagggatcg

SEQ ID NO: 665 - DNA, 50 bp, synthetic construct
  source 1..50: mol_type = other DNA
cgcgcccattggtgacccttccaggcagacagcctcagcaacgcccctgg

SEQ ID NO: 666 - DNA, 50 bp, synthetic construct
  source 1..50: mol_type = other DNA
cgctggcacagagaacacaattatgcacagccttgaaaaattgtatcata

SEQ ID NO: 667 - DNA, 50 bp, synthetic construct
  source 1..50: mol_type = other DNA
cgcggagggcagagcagtgacctccaatgatttacaggcctttagcttaa

SEQ ID NO: 668 - DNA, 50 bp, synthetic construct
  source 1..50: mol_type = other DNA
cggttggaagccaagcaacgggaagacatctgggaaggcagagaccagtc

SEQ ID NO: 669 - DNA, 50 bp, synthetic construct
  source 1..50: mol_type = other DNA
cgggcacttaagcagctaggctgatggtccctaatgaaatcaaataaaat

SEQ ID NO: 670 - DNA, 50 bp, synthetic construct
  source 1..50: mol_type = other DNA
cgggagcagtaaattggagcagtttgcttcaatcttgctgcaagttgaaa

SEQ ID NO: 671 - DNA, 50 bp, synthetic construct
  source 1..50: mol_type = other DNA
cggccgacgctgagatccatgccattgtagccgtagccgtacctgccgga

SEQ ID NO: 672 - DNA, 50 bp, synthetic construct
  source 1..50: mol_type = other DNA
gaggttctgtatatcattcaaagaccatctgtcgtgcaattcctgtagcg

SEQ ID NO: 673 - DNA, 50 bp, synthetic construct
  source 1..50: mol_type = other DNA
cgctctgttttttgaggaacctgaatgaacctgaaagcaaagaccgagtg

SEQ ID NO: 674 - DNA, 50 bp, synthetic construct
  source 1..50: mol_type = other DNA
tctaacgcaaattttccttgctctgctttgccaacaggagtgtaaacacg

SEQ ID NO: 675 - DNA, 50 bp, synthetic construct
  source 1..50: mol_type = other DNA
ttttcctctcagctatcaaatgtcactgccttgaaacgtgggccctttcg

SEQ ID NO: 676 - DNA, 50 bp, synthetic construct
  source 1..50: mol_type = other DNA
gagtctaaacttttcccctgaggtcaagaatgtggctggtccccaagccg

SEQ ID NO: 677 - DNA, 50 bp, synthetic construct
  source 1..50: mol_type = other DNA
cgatgtttcctgagattgaaaaatgattttctaccttaaacagtggtttc

SEQ ID NO: 678 - DNA, 50 bp, synthetic construct
  source 1..50: mol_type = other DNA
cgcacaagtatcctgcatgttccgattctgactggcacctggagcggctc

SEQ ID NO: 679 - DNA, 50 bp, synthetic construct
  source 1..50: mol_type = other DNA
cgacgtgccacaaatggtattttactctcaataagaactcgagctgagtt

SEQ ID NO: 680 - DNA, 50 bp, synthetic construct
  source 1..50: mol_type = other DNA
ttctgctaactttcagacttcattgctgacaacaggcctcttggctttcg

SEQ ID NO: 681 - DNA, 50 bp, synthetic construct
  source 1..50: mol_type = other DNA
gtgccatttgtaaaatcagttcactttcttttatttattactgcagtgcg

SEQ ID NO: 682 - DNA, 50 bp, synthetic construct
  source 1..50: mol_type = other DNA
cgggcagtggggccagtgtctttatcggccagatggtgtgaatttagaca

SEQ ID NO: 683 - DNA, 50 bp, synthetic construct
  source 1..50: mol_type = other DNA
tgtataagatctgacagcccggcagttataggccacctgcatcactgtcg

SEQ ID NO: 684 - DNA, 50 bp, synthetic construct
  source 1..50: mol_type = other DNA
catataatctcactaacgctggactccatttgactcttttgcaggctacg

SEQ ID NO: 685 - DNA, 50 bp, synthetic construct
  source 1..50: mol_type = other DNA
ctagctcttgctcagagttgaggccttggtcgggtatgtgtgcgtgcgcg

SEQ ID NO: 686 - DNA, 50 bp, synthetic construct
  source 1..50: mol_type = other DNA
cgaatggcaatcttacgctttcttgcagcctgtgggttgacattaataaa

SEQ ID NO: 687 - DNA, 50 bp, synthetic construct
  source 1..50: mol_type = other DNA
cttggctttcctcattccctctcctcattaatttgtgtgagctcagctcg

SEQ ID NO: 688 - DNA, 50 bp, synthetic construct
  source 1..50: mol_type = other DNA
cggtgggcttaacctctgctattaatctgtacaagtgcctcctgggcttt

SEQ ID NO: 689 - DNA, 50 bp, synthetic construct
  source 1..50: mol_type = other DNA
cgacctgcacctgtctgtgaagtatgtagggtgggcagaagccacagtcg

SEQ ID NO: 690 - DNA, 50 bp, synthetic construct
  source 1..50: mol_type = other DNA
tgtttctctaatctgcaattatttatgttttcttcatgctgctttgtccg

SEQ ID NO: 691 - DNA, 50 bp, synthetic construct
  source 1..50: mol_type = other DNA
cgctggcggctttattgaaggaataaactatatttgcaaagtaagatatt

SEQ ID NO: 692 - DNA, 50 bp, synthetic construct
  source 1..50: mol_type = other DNA
ttttcttttatacatagctttatcctataattttgtccctgttgggcacg

SEQ ID NO: 693 - DNA, 50 bp, synthetic construct
  source 1..50: mol_type = other DNA
gacaaaggcacaatgaaatagaaagcttaccaccggtagctttcaaaacg

SEQ ID NO: 694 - DNA, 50 bp, synthetic construct
  source 1..50: mol_type = other DNA
cgcagtccggcgttccctggggaaccgttcaactcatgaggtctgatttc

SEQ ID NO: 695 - DNA, 50 bp, synthetic construct
  source 1..50: mol_type = other DNA
cggatggttaataggggagataacaggattcataagggaaatttatcggt

SEQ ID NO: 696 - DNA, 50 bp, synthetic construct
  source 1..50: mol_type = other DNA
cggttcgcagctaatgagcatgcagctggattgcaaatggctggatttat

SEQ ID NO: 697 - DNA, 50 bp, synthetic construct
  source 1..50: mol_type = other DNA
ctctgtagcgccaagttcaaaatcagctcggacttgaaaaggcacatgcg

SEQ ID NO: 698 - DNA, 50 bp, synthetic construct
  source 1..50: mol_type = other DNA
taaaacaattttaaagtgattaagtagtttaacacctgtctcccgtcacg

SEQ ID NO: 699 - DNA, 50 bp, synthetic construct
  source 1..50: mol_type = other DNA
attgaaaactcattacaagctgtttagactctgactttgacttttgctcg

SEQ ID NO: 700 - DNA, 50 bp, synthetic construct
  source 1..50: mol_type = other DNA
cgtgacatctccatggctgtaccaccttgtcgggtagcttatcagactga

SEQ ID NO: 701 - DNA, 50 bp, synthetic construct
  source 1..50: mol_type = other DNA
tgggcccataccaaccttgtttagtcccacagtgttttgttcgccccacg

SEQ ID NO: 702 - DNA, 50 bp, synthetic construct
  source 1..50: mol_type = other DNA
cgggcaaaagcctgtaattctatttgttttgggtaaatttagcaagctgc

SEQ ID NO: 703 - DNA, 50 bp, synthetic construct
  source 1..50: mol_type = other DNA
caaacctttgtcactgttcctaatgcactcttaggcctcattaatctccg

SEQ ID NO: 704 - DNA, 50 bp, synthetic construct
  source 1..50: mol_type = other DNA
ctttcttctggtggtagcgggcctggcagcaggactccaggtagatctcg

SEQ ID NO: 705 - DNA, 50 bp, synthetic construct
  source 1..50: mol_type = other DNA
cgtcctagctgtcagcaggcaaatgctttcattttggtctcaggcaaggg

SEQ ID NO: 706 - DNA, 50 bp, synthetic construct
  source 1..50: mol_type = other DNA
ggccaatcaggagcgcctctccagatacaaagcatcccaatcaggcggcg

SEQ ID NO: 707 - DNA, 50 bp, synthetic construct
  source 1..50: mol_type = other DNA
agcattaattaagactgtaaatcacaggccacatgaaacaactgtatacg

SEQ ID NO: 708 - DNA, 50 bp, synthetic construct
  source 1..50: mol_type = other DNA
tgtgtttttcaaatattgtctccccaggcaggctggctgttgggtaaacg

SEQ ID NO: 709 - DNA, 50 bp, synthetic construct
  source 1..50: mol_type = other DNA
cgccttgggtaaaaagcaatgcatacgtttacaatataaatgtgatttaa

SEQ ID NO: 710 - DNA, 50 bp, synthetic construct
  source 1..50: mol_type = other DNA
cgaggtcgccttcccagcagcagtgaggattctgacgacaatggcagtaa

SEQ ID NO: 711 - DNA, 50 bp, synthetic construct
  source 1..50: mol_type = other DNA
cgcatttatatgctacaggttacaaagtgagagccttgtatacacttcaa

SEQ ID NO: 712 - DNA, 50 bp, synthetic construct
  source 1..50: mol_type = other DNA
cgtcattttgatgtcacgtttccatagcaacagagcaactgcataagcta

SEQ ID NO: 713 - DNA, 50 bp, synthetic construct
  source 1..50: mol_type = other DNA
tttcacaaggtttttttttcagccgatccagctggccggaatgaatagcg

SEQ ID NO: 714 - DNA, 50 bp, synthetic construct
  source 1..50: mol_type = other DNA
gtttcaatgtacttttttagttcttccatctttttcttgtgtacttttcg

SEQ ID NO: 715 - DNA, 50 bp, synthetic construct
  source 1..50: mol_type = other DNA
gtaaaacgtcaagggcagtaatgcgattagcggtctcaaatgaaatatcg

SEQ ID NO: 716 - DNA, 50 bp, synthetic construct
  source 1..50: mol_type = other DNA
cgagccagaaggaagactcacaaagatttcaacatacttctggtaggtag

SEQ ID NO: 717 - DNA, 50 bp, synthetic construct
  source 1..50: mol_type = other DNA
ttatttgcggctgtcggttcccagtagaaaaatttcgtcttggagagccg

SEQ ID NO: 718 - DNA, 50 bp, synthetic construct
  source 1..50: mol_type = other DNA
cgctgtcactcaagggctttcaagtcagcgatgtagaaatcaaaaagaaa

SEQ ID NO: 719 - DNA, 50 bp, synthetic construct
  source 1..50: mol_type = other DNA
cgccaactgctgggtgccatttaaaccctccccgctggcatggaaccaga

SEQ ID NO: 720 - DNA, 50 bp, synthetic construct
  source 1..50: mol_type = other DNA
cacacatatcaaaaaacaaatgagctcttattttgtaaactcattttgcg

SEQ ID NO: 721 - DNA, 50 bp, synthetic construct
  source 1..50: mol_type = other DNA
ctcagtccctcttgtgttcacagttgggcaaggcgagcatcatggcctcg

SEQ ID NO: 722 - DNA, 50 bp, synthetic construct
  source 1..50: mol_type = other DNA
cgccacttgattggtttgggatttccaggacatggagttttgaatttgca

SEQ ID NO: 723 - DNA, 50 bp, synthetic construct
  source 1..50: mol_type = other DNA
gttattccactctgcaccaactataaaaaaatactatcaaagtttgaccg

SEQ ID NO: 724 - DNA, 50 bp, synthetic construct
  source 1..50: mol_type = other DNA
cggcagttcaaagggaatatttcaacaatgattacattttgtaaatcttt

SEQ ID NO: 725 - DNA, 50 bp, synthetic construct
  source 1..50: mol_type = other DNA
cgtcatcctgtgctgacattttaatcgtgcttttgtgggagctggctggc

SEQ ID NO: 726 - DNA, 50 bp, synthetic construct
  source 1..50: mol_type = other DNA
cgactgagctcaaatttcagttggggccctattagtctgcctttccaaaa

SEQ ID NO: 727 - DNA, 50 bp, synthetic construct
  source 1..50: mol_type = other DNA
cggattatataaacctgtccacctatcaagtgttttagacaacagaacag

SEQ ID NO: 728 - DNA, 50 bp, synthetic construct
  source 1..50: mol_type = other DNA
cgggtaatgacagtctgggctaatgaaagaggcttctcttctggcaaaaa

SEQ ID NO: 729 - DNA, 50 bp, synthetic construct
  source 1..50: mol_type = other DNA
cacaggtcaatagctgaagtcacacctttgccagctttgattctttctcg

SEQ ID NO: 730 - DNA, 50 bp, synthetic construct
  source 1..50: mol_type = other DNA
aacacagcggccaataagaattatttgccttattaaagcagcagaagacg

SEQ ID NO: 731 - DNA, 50 bp, synthetic construct
  source 1..50: mol_type = other DNA
cagcagctgaatttttcaggctaatttgatcctccacactgagacgatcg

SEQ ID NO: 732 - DNA, 50 bp, synthetic construct
  source 1..50: mol_type = other DNA
ttcggaatacttcttgcaataatagtgcatatagctcaatcccttaaccg

SEQ ID NO: 733 - DNA, 50 bp, synthetic construct
  source 1..50: mol_type = other DNA
cgtcttggagaacctgatggttttgattgccatctggaaaaacaataaat

SEQ ID NO: 734 - DNA, 50 bp, synthetic construct
  source 1..50: mol_type = other DNA
ttacataagtcctggctcccttatttagccccagaaccaattcgttatcg

SEQ ID NO: 735 - DNA, 50 bp, synthetic construct
  source 1..50: mol_type = other DNA
cgggcctctggctgtgctctgtagctgtgaaatagaatcaagattccatt

SEQ ID NO: 736 - DNA, 50 bp, synthetic construct
  source 1..50: mol_type = other DNA
cggtgttgcctgtctgcgccggccattgttgcaatgtaagcaatactgtt

SEQ ID NO: 737 - DNA, 50 bp, synthetic construct
  source 1..50: mol_type = other DNA
caatgtagacatctctgaaaaggtcaacttgacacaggctctgacagtcg

SEQ ID NO: 738 - DNA, 50 bp, synthetic construct
  source 1..50: mol_type = other DNA
cctagtagagacaccagaccttttcaaacaagggacaacaaggaaaatcg

SEQ ID NO: 739 - DNA, 50 bp, synthetic construct
  source 1..50: mol_type = other DNA
agagaggtgggcctttgatctctttgttgcagctgctccctgggaactcg

SEQ ID NO: 740 - DNA, 50 bp, synthetic construct
  source 1..50: mol_type = other DNA
ctctctccagagcctcagctcaaaggcatcgtcaccaaactgttctgccg

SEQ ID NO: 741 - DNA, 50 bp, synthetic construct
  source 1..50: mol_type = other DNA
tttattctctcctcctatcagctattagtgcctaatcatatggtctatcg

SEQ ID NO: 742 - DNA, 50 bp, synthetic construct
  source 1..50: mol_type = other DNA
gtttcccattacatcacaaaggagctcaggcagatctctgcacagaaacg

SEQ ID NO: 743 - DNA, 50 bp, synthetic construct
  source 1..50: mol_type = other DNA
gcggcagcagtggcagcaggaaccgaacatccaaaatggcggctccctcg

SEQ ID NO: 744 - DNA, 50 bp, synthetic construct
  source 1..50: mol_type = other DNA
actcaaataacaatgttgtcctcccggtcgggctatgtgtcactcactcg

SEQ ID NO: 745 - DNA, 50 bp, synthetic construct
  source 1..50: mol_type = other DNA
cggctggacctggtcatggtgattttgtttaaggggatccccctggaaag

SEQ ID NO: 746 - DNA, 50 bp, synthetic construct
  source 1..50: mol_type = other DNA
gtggcctgagcacggagtgagctcgaaggcttgagagcggcctctgcgcg

SEQ ID NO: 747 - DNA, 50 bp, synthetic construct
  source 1..50: mol_type = other DNA
cggaagtctatgtggagcagtctggctagccccagtctaacctccccatc

SEQ ID NO: 748 - DNA, 50 bp, synthetic construct
  source 1..50: mol_type = other DNA
cactaaatgcaaacggcggtccctgctgttcccatcaaattaattaaccg

SEQ ID NO: 749 - DNA, 50 bp, synthetic construct
  source 1..50: mol_type = other DNA
ctgacctttaatctgatgctagtttactctcatcactgaagtggaagtcg

SEQ ID NO: 750 - DNA, 50 bp, synthetic construct
  source 1..50: mol_type = other DNA
cgcctccagctgcgagtgtctggtggaggcactttgtaaaatcatgaaaa

SEQ ID NO: 751 - DNA, 50 bp, synthetic construct
  source 1..50: mol_type = other DNA
catttaaaccctccccgctggcatggaaccagagataaatatagccaacg

SEQ ID NO: 752 - DNA, 50 bp, synthetic construct
  source 1..50: mol_type = other DNA
cggtgcctgcttgattccagctattttgcgtttcaatgtggcatcagaat

SEQ ID NO: 753 - DNA, 50 bp, synthetic construct
  source 1..50: mol_type = other DNA
cggctcttttgttggaactatttagaggttattaagatgtgtcttcagaa

SEQ ID NO: 754 - DNA, 50 bp, synthetic construct
  source 1..50: mol_type = other DNA
cggcagccagtgcatgcagcccaacaacattatgggcatcgagaacatct

SEQ ID NO: 755 - DNA, 30 bp, synthetic construct
  source 1..30: mol_type = other DNA
agttttaggaaaagaaaggatgtaaatgtg

SEQ ID NO: 756 - DNA, 29 bp, synthetic construct
  source 1..29: mol_type = other DNA
caaattaaaccaacaaaaccataactacc

(19) United States
(12) Patent Application Publication
(10) Pub. No.: US 2026/0015652 A1
(43) Pub. Date: Jan. 15, 2026

(54) METHYLATION FOR MONITORING CIRCANNUAL RHYTHMS

(71) Applicant: University of South Carolina, Columbia, SC (US)

(72) Inventors: Hippokratis Kiaris, Irmo, SC (US); Kim Tuyen Huynh Dam, Columbia, SC (US)

(73) Assignee: University of South Carolina, Columbia, SC (US)

(21) Appl. No.: 19/268,293

(22) Filed: Jul. 14, 2025

Related U.S. Application Data
(60) Provisional application No. 63/670,324, filed on Jul. 12, 2024.

(51) Int. Cl. C12Q 1/6827; C12Q 1/6806

(52) U.S. Cl. CPC C12Q 1/6827; C12Q 1/6806; C12Q 2600/154

(57) ABSTRACT

Methods for monitoring circannual rhythms and/or determining birth period by detecting DNA methylation and oligonucleotide arrays therefor.

### CROSS-REFERENCE TO RELATED APPLICATIONS

Priority is hereby claimed to U.S. Provisional Application 63/670,324, filed Jul. 12, 2024, which is incorporated herein by reference in its entirety.

### STATEMENT REGARDING FEDERALLY SPONSORED RESEARCH

This invention was made with government support under OIA1736150 awarded by the National Science Foundation. The government has certain rights in the invention.

### SEQUENCE LISTING

The instant application contains a Sequence Listing which has been submitted in XML format and is hereby incorporated by reference in its entirety. The XML copy, created on Jul. 11, 2025, is named USPTO--250711--106479.048--SEQ_LIST.xml and is 718,284 bytes in size.

### FIELD OF THE INVENTION

Provided herein are methods for monitoring circannual rhythms and/or determining birth month by detecting DNA methylation and oligonucleotide arrays therefor.

### BACKGROUND

Circadian (circannual) rhythms play an increasingly appreciated role in mental health and physical well-being. We report a list of genes and gene sequences regulated by epigenetic mechanisms that are involved in circannual rhythms. These genes and sequences represent a powerful platform for discovery in genetics and epigenetics, particularly for monitoring circannual rhythms and other traits and/or for detecting birth month.

### SUMMARY OF THE INVENTION

One aspect of the invention is directed to methods of determining a methylation level of any one or more CpG sites in one or more genomic regions of a genome of a subject.

In some versions, the methods comprise determining a methylation level of any one or more CpG sites in each of 10 or more genomic regions of a genome of a subject. In some versions, the genomic regions have sequences of any 10 or more of SEQ ID NOS:1-377 or sequences at least 80% identical thereto. In some versions, the genomic regions have sequences of any 10 or more of SEQ ID NOS:1-25 or sequences at least 80% identical thereto.

In some versions, the methods comprise determining a methylation level of any one or more CpG sites in each of 25 or more genomic regions of the genome of the subject, wherein the genomic regions have sequences of each of SEQ ID NOS:1-25 or sequences at least 80% identical thereto.

In some versions, the methods comprise determining a methylation level of any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 10 of SEQ ID NOS:1-25 or sequences at least 80% identical thereto.

In some versions, the methods comprise determining a methylation level of any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 15 of SEQ ID NOS:1-25 or sequences at least 80% identical thereto.

In some versions, the methods comprise determining a methylation level of any one or more CpG sites in each of 100 or more genomic regions of the genome of the subject, wherein the genomic regions have sequences of any 100 or more of SEQ ID NOS:1-377 or sequences at least 80% identical thereto, with the proviso that the genomic regions have sequences of at least 25 of SEQ ID NOS:1-50 or sequences at least 80% identical thereto.

In some versions, a methylation level is determined for no more than 30,000 CpG sites in the genome of the subject.

In some versions, the determining comprises: treating genomic DNA from the subject with bisulfite to generate bisulfite-treated genomic DNA; amplifying the bisulfite-treated genomic DNA using primers that amplify portions of the bisulfite-treated genomic DNA comprising the genomic regions; and measuring the methylation level of the one or more CpG sites in each of the genomic regions. In some versions, the determining comprises: treating genomic DNA from the subject with bisulfite to generate bisulfite-treated genomic DNA; amplifying the bisulfite-treated genomic DNA using primers specific for portions of the bisulfite-treated genomic DNA comprising the genomic regions; and measuring the methylation level of the one or more CpG sites in each of the genomic regions. In some versions, the portion of the bisulfite-treated genomic DNA has a length less than 1000 bases.

In some versions, the methylation level is measured by methylation-specific PCR, quantitative methylation-specific PCR, methylation-sensitive DNA restriction enzyme analysis, or bisulfite genomic sequencing PCR, or quantitative bisulfite pyrosequencing.

In some versions, the methods further comprise determining from the methylation level of the one or more CpG sites a birth period of the subject.

Another aspect of the invention is directed to arrays of immobilized oligonucleotides, wherein the oligonucleotides are configured to bind to amplicons of bisulfite modified genomic DNA.

In some versions, the oligonucleotides are configured to bind to amplicons comprising bisulfite modified CpG sites corresponding to any one or more CpG sites in each of 10 or more genomic regions of a genome of a subject, wherein the genomic regions have sequences of any 10 or more of SEQ ID NOS:1-377 or sequences at least 80% identical thereto.

In some versions, the oligonucleotides are configured to bind to amplicons comprising bisulfite modified CpG sites corresponding to any one or more CpG sites in each of 10 or more genomic regions of the genome of the subject, wherein the genomic regions have sequences of any 10 or more of SEQ ID NOS:1-25 or sequences at least 80% identical thereto.

In some versions the oligonucleotides are configured to bind to amplicons comprising bisulfite modified CpG sites corresponding to any one or more CpG sites in each of 10 or more genomic regions of the genome of the subject, wherein the genomic regions have sequences of each of SEQ ID NOS:1-25 or sequences at least 80% identical thereto.

In some versions, the oligonucleotides are configured to bind to amplicons comprising bisulfite modified CpG sites corresponding to any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 10 of SEQ ID NOS:1-25 or sequences at least 80% identical thereto.

In some versions, the oligonucleotides are configured to bind to amplicons comprising bisulfite modified CpG sites corresponding to any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 15 of SEQ ID NOS:1-25 or sequences at least 80% identical thereto.

In some versions, the oligonucleotides are configured to bind to amplicons comprising bisulfite modified CpG sites corresponding to any one or more CpG sites in each of 100 or more genomic regions of the genome of the subject, wherein the genomic regions have sequences of any 100 or more of SEQ ID NOS:1-377 or sequences at least 80% identical thereto, with the proviso that the genomic regions have sequences of at least 25 of SEQ ID NOS:1-50 or sequences at least 80% identical thereto.

In some versions, the array comprises no more than 30,000 different oligonucleotides.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Distribution of mice (n=96) used for the methylation analysis in relation to their birth month. Males and females are indicated.

FIGS. 2A and 2B. CpGs following circannual rhythm in methylation in Peromyscus. Biological function prediction for CpGs exhibiting circannual methylation (sorted by FDR). FIG. 2A. Bar graphs. FIG. 2B. biological function networks.

FIGS. 3A and 3B. Circannual rhythm in methylation in Peromyscus. Scatter plots showing relative methylation of CpGs that exhibit significant demethylation (FIG. 3A) or hypermethylation (FIG. 3B) in September that were identified by using a simple sinusoidal model.

FIGS. 4A-4F. Circannual methylation in representative CpGs in polygamous (FIGS. 4A-4C) or both polygamous and monogamous (FIGS. 4D-4F) Peromyscus stocks. Stock name is indicated in the right.

FIGS. 5A-5E. Differential methylation in Peromyscus according to birth season. FIG. 5A. Pie diagram showing the percentage of the CpGs at which highest, lowest, or intermediate degree of methylation is recorded in September. FIG. 5B. Scatter plot indicating the P value of fitness in a sinusoidal model for all 37,000 different CpGs for females (blue, n=50), and males (red, n=46). Top 1000 CpGs ranked by −Log P are shown in the inset. FIG. 5C. Scatter plots showing relative methylation of CpGs that were identified in females by using a simple sinusoidal model (P<0.05, FDR<0.1). CpG IDs and gene names are indicated. FIG. 5D. Scatter plot indicating the P value of fitness in a sinusoidal model for different CpGs for all (blue), the monogamous (red), and the polygamous (green) Peromyscus stocks. FIG. 5E. Top 1000 CpGs ranked by log P.

FIG. 6. Position of CpGs that are differentially methylated in a season-specific manner in polygamous and monogamous Peromyscus.

FIGS. 7A-7F. Biological functions associated with season-dependent methylation. FIG. 7A. Venn diagram with the genes that are differentially methylated according to season in monogamous and polygamous species (P<0.05). The biological functions (KEGG) of the corresponding gene sets are indicated in bar plots that also show enriched gene number and enrichment confidence (FDR). FIGS. 7B-7F. Differential methylation of selected gene sets that are associated with specific biological processes. The integration of these genes in the corresponding processes is shown.

### DETAILED DESCRIPTION OF THE INVENTION

One aspect of the invention is directed to methods of determining methylation level.

Some versions comprise determining a methylation level of any one or more CpG sites in each of 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, 275 or more, 300 or more, 325 or more, 350 or more, or 375 or more genomic regions of a genome of a subject.

In various versions of the invention, the genomic regions can have sequences of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, 275 or more, 300 or more, 325 or more, 350 or more, or 375 or more of SEQ ID NOS:1-377 or sequences at least 80% identical thereto.

In various versions of the invention, the genomic regions can have sequences of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, 275 or more, or each of SEQ ID NOS:1-300 or sequences at least 80% identical thereto.

In various versions of the invention, the genomic regions can have sequences of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, or each of SEQ ID NOS:1-250 or sequences at least 80% identical thereto.

In various versions of the invention, the genomic regions can have sequences of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, or each of SEQ ID NOS:1-200 or sequences at least 80% identical thereto.

In various versions of the invention, the genomic regions can have sequences of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, or each of SEQ ID NOS:1-150 or sequences at least 80% identical thereto.

In various versions of the invention, the genomic regions can have sequences of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, or each of SEQ ID NOS:1-100 or sequences at least 80% identical thereto.

In various versions of the invention, the genomic regions can have sequences of any 5 or more, 10 or more, 15 or more, 20 or more, 25 or more, 30 or more, 35 or more, 40 or more, 45 or more, or each of SEQ ID NOS:1-50 or sequences at least 80% identical thereto.

In various versions of the invention, the genomic regions can have sequences of any 1 or more, any 2 or more, any 3 or more, any 4 or more, any 5 or more, any 6 or more, any 7 or more, any 8 or more, any 9 or more, 10 or more, 11 or more, any 12 or more, any 13 or more, any 14 or more, any 15 or more, any 16 or more, any 17 or more, any 18 or more, any 19 or more, 20 or more, 21 or more, any 22 or more, any 23 or more, any 24 or more, or each of SEQ ID NOS:1-25 or sequences at least 80% identical thereto.

In various versions of the invention, the genomic regions can have sequences of any 1 or more, any 2 or more, any 3 or more, any 4 or more, any 5 or more, any 6 or more, any 7 or more, any 8 or more, any 9 or more, 10 or more, 11 or more, any 12 or more, any 13 or more, any 14 or more, any 15 or more, any 16 or more, any 17 or more, any 18 or more, any 19 or more, or each of SEQ ID NOS:1-20 or sequences at least 80% identical thereto.

In various versions of the invention, the above-referenced genomic regions can have sequences of any 1 or more, any 2 or more, any 3 or more, any 4 or more, any 5 or more, any 6 or more, any 7 or more, any 8 or more, any 9 or more, 10 or more, 11 or more, any 12 or more, any 13 or more, any 14 or more, or each of SEQ ID NOS:1-15 or sequences at least 80% identical thereto.

In various versions of the invention, the above-referenced genomic regions can have sequences of any 1 or more, any 2 or more, any 3 or more, any 4 or more, any 5 or more, any 6 or more, any 7 or more, any 8 or more, any 9 or more, or each of SEQ ID NOS:1-10 or sequences at least 80% identical thereto.

In various versions of the invention, the above-referenced genomic regions can have sequences of any 1 or more, any 2 or more, any 3 or more, any 4 or more, or each of SEQ ID NOS:1-5 or sequences at least 80% identical thereto.

In various versions of the invention, the above-referenced genomic regions can have sequences of any 1 or more, any 2 or more, or each of SEQ ID NOS:1-3 or sequences at least 80% identical thereto.

The sequences at least 80% identical to the aforementioned SEQ ID NOS can be at least 85%, at least 90%, at least 95%, at least 99%, or 100% identical thereto.

As used herein, recitations such as “SEQ ID NOS:1-377 or sequences at least 80% identical thereto” refer to “SEQ ID NO:1 or a sequence at least 80% identical thereto, SEQ ID NO:2 or a sequence at least 80% identical thereto, SEQ ID NO:3 or a sequence at least 80% identical thereto . . . .” Similarly, recitations such as “2 or more of SEQ ID NOS:1-5 or sequences at least 80% identical thereto” refer to “2 or more of SEQ ID NO:1 or a sequence at least 80% identical thereto, SEQ ID NO:2 or a sequence at least 80% identical thereto, SEQ ID NO:3 or a sequence at least 80% identical thereto, SEQ ID NO:4 or a sequence at least 80% identical thereto, and SEQ ID NO:5 or a sequence at least 80% identical thereto.” In other words, each of the specified number of sequences (e.g., 2 or more) must either be a different specified SEQ ID NO or a variant of a different specified SEQ ID NO, such that no single SEQ ID NO either in exact form or variant form is represented more than once.

In some embodiments, the methylation level is determined for no more than 800,000 CpG sites, no more than 700,000 CpG sites, no more than 600,000 CpG sites, no more than 500,000 CpG sites, no more than 400,000 CpG sites, no more than 300,000 CpG sites, no more than 250,000 CpG sites, no more than 200,000 CpG sites, no more than 150,000 CpG sites, no more than 100,000 CpG sites, no more than 75,000 CpG sites, no more than 50,000 CpG sites, no more than 30,000 CpG sites, no more than 25,000 CpG sites, no more than 10,000 CpG sites, no more than 5,000 CpG sites, no more than 2,500 CpG sites, no more than 1,000 CpG sites, no more than 750 CpG sites, no more than 500 CpG sites, no more than 400 CpG sites, no more than 300 CpG sites, no more than 200 CpG sites, no more than 100 CpG sites, no more than 75 CpG sites, no more than 50 CpG sites, or no more than 25 CpG sites in the genome of the subject.

In some embodiments, the methylation level is determined for no more than 800,000 separate genomic regions, no more than 700,000 separate genomic regions, no more than 600,000 separate genomic regions, no more than 500,000 separate genomic regions, no more than 400,000 separate genomic regions, no more than 300,000 separate genomic regions, no more than 250,000 separate genomic regions, no more than 200,000 separate genomic regions, no more than 150,000 separate genomic regions, no more than 100,000 separate genomic regions, no more than 75,000 separate genomic regions, no more than 50,000 separate genomic regions, no more than 30,000 separate genomic regions, no more than 25,000 separate genomic regions, no more than 10,000 separate genomic regions, no more than 5,000 separate genomic regions, no more than 2,500 separate genomic regions, no more than 1,000 separate genomic regions, no more than 750 separate genomic regions, no more than 500 separate genomic regions, no more than 400 separate genomic regions, no more than 300 separate genomic regions, no more than 200 separate genomic regions, no more than 100 separate genomic regions, no more than 75 separate genomic regions, no more than 50 separate genomic regions, or no more than 25 separate genomic regions in the genome of the subject. Genomic regions are considered be “separate” in this context when they have at least one base separating each other.

Some versions comprise determining from the methylation level of the one or more CpG sites a birth period of the subject. The birth period can be any span of time within the course of a year. Exemplary spans of time include 1 day, 5 days, 10 days, 15 days, 20 days, 30 days, 35 days, 40 days, 50 days, 60 days, 70 days, 80 days, 90 days, 100 days, 120 days, 140 days, 160 days, 180 days, 200 days, 220 days, 240 days, 260 days, 280 days, 300 days, 320 days, 240 days, or 30 days. In some versions, the birth period is birth month, such that the birth month of the animal is determined. The birth period can be determined by correlating the methylation patterns of the one or more CpG sites with the methylation patterns of animals born throughout the year, as shown in the examples.

Another aspect of the invention is directed to arrays of immobilized oligonucleotides. The oligonucleotides can be immobilized on a solid surface. The oligonucleotides can be immobilized on the solid surface in distinct patterns, such as with groups of oligonucleotides having identical sequences immobilized together on the solid surface and away from oligonucleotides having distinct sequences.

In some versions, the oligonucleotides are configured to bind to amplicons of bisulfite modified genomic DNA. The amplicons to which the oligonucleotides are configured to bind can be amplicons of bisulfite modified forms of any one or more of the genomic regions described herein or portions thereof. The portions thereof can include 5 or more, 10 or more 15 or more, 20 or more, 25 or more, 30 or more, 35 or more, 40 or more, or each of the bases in the regions described herein. The amplicons preferably comprise one or more bisulfite modified CpG sites corresponding to one or more the CpG sites of the genomic regions described herein. The correspondence of a bisulfite modified CpG site to a CpG site of the genomic regions described herein can be recognized by the alignment of the amplicon comprising the bisulfite modified CpG site to the CpG site of the genomic region. As used herein, a CpG site is considered to be “bisulfite modified” if it is included in or is amplified from DNA that has been bisulfite modified, whether or not its sequence is modified. It is recognized that the sequence of CpG site in the amplicon will be CG (for the reverse complement) if the CpG site was methylated in the original genomic DNA and T/UG (CA for the reverse complement) if the CpG site was unmethylated in the original genomic DNA. “CpG site” in the context of the amplicon refers to either strand in an amplicon (i.e., the sense or antisense strand).

The oligonucleotides in the arrays of the invention can be configured to bind to amplicons comprising bisulfite modified CpG sites corresponding to any one or more of the CpG sites outlined by the CPG IDs in Table 2 below, in any combination. Accordingly, the amplicons will have bisulfite modified CpG sites, including portions of the sequence upstream and/or downstream of the CpG sites, and the oligonucleotides will have sequences that bind to these upstream and/or downstream portions. The oligonucleotides in the arrays can comprise sequences that bind to amplified bisulfite modified genomic sequences within 500 bp of the CpG sites, within 475 bp of the CpG sites, within 450 bp of the CpG sites, within 425 bp of the CpG sites, within 400 bp of the CpG sites, within 375 bp of the CpG sites, within 350 bp of the CpG sites, within 325 bp of the CpG sites, within 300 bp of the CpG sites, within 275 bp of the CpG sites, within 250 bp of the CpG sites, within 225 bp of the CpG sites, within 200 bp of the CpG sites, within 175 bp of the CpG sites, within 150 bp of the CpG sites, within 125 bp of the CpG sites, within 100 bp of the CpG sites, within 75 bp of the CpG sites, within 50 bp of the CpG sites, within 25 bp of the CpG sites, within 10 bp of the CpG sites, or within 5 bp of the CpG sites. “Amplified bisulfite modified genomic sequences” refers to sequences in amplicons of bisulfite modified genomic DNA. The detection of methylation can occur by pyrosequencing or other methods known in the art of methylation arrays.

In some versions of the invention, the oligonucleotides in the arrays of the invention can be configured to bind amplicons comprising bisulfite modified CpG sites corresponding to any one or more CpG sites in each of 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, 275 or more, 300 or more, 325 or more, 350 or more, or 375 or more genomic regions of a genome of a subject. The genomic regions can have any number and/or combination of sequences described above for the methods of the invention.

In various versions of the invention, the oligonucleotides in the arrays can be configured to bind at least a portion of a bisulfite modified sequence of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, 275 or more, 300 or more, 325 or more, 350 or more, or 375 or more of SEQ ID NOS:1-377 or sequences at least 80% identical thereto.

In various versions of the invention, the oligonucleotides in the arrays can be configured to bind at least a portion of a bisulfite modified sequence of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, 275 or more, or each of SEQ ID NOS:1-300 or sequences at least 80% identical thereto.

In various versions of the invention, the oligonucleotides in the arrays can be configured to bind at least a portion of a bisulfite modified sequence of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, 200 or more, 225 or more, 250 or more, or each of SEQ ID NOS:1-250 or sequences at least 80% identical thereto.

In various versions of the invention, the oligonucleotides in the arrays can be configured to bind at least a portion of a bisulfite modified sequence of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, 150 or more, 175 or more, or each of SEQ ID NOS:1-200 or sequences at least 80% identical thereto.

In various versions of the invention, the oligonucleotides in the arrays can be configured to bind at least a portion of a bisulfite modified sequence of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 125 or more, or each of SEQ ID NOS:1-150 or sequences at least 80% identical thereto.

In various versions of the invention, the oligonucleotides in the arrays can be configured to bind at least a portion of a bisulfite modified sequence of any 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, or each of SEQ ID NOS:1-100 or sequences at least 80% identical thereto.

In various versions of the invention, the oligonucleotides in the arrays can be configured to bind at least a portion of a bisulfite modified sequence of any 5 or more, 10 or more, 15 or more, 20 or more, 25 or more, 30 or more, 35 or more, 40 or more, 45 or more, or each of SEQ ID NOS:1-50 or sequences at least 80% identical thereto.

In various versions of the invention, the oligonucleotides in the arrays can be configured to bind at least a portion of a bisulfite modified sequence of any 1 or more, any 2 or more, any 3 or more, any 4 or more, any 5 or more, any 6 or more, any 7 or more, any 8 or more, any 9 or more, 10 or more, 11 or more, any 12 or more, any 13 or more, any 14 or more, any 15 or more, any 16 or more, any 17 or more, any 18 or more, any 19 or more, 20 or more, 21 or more, any 22 or more, any 23 or more, any 24 or more, or each of SEQ ID NOS:1-25 or sequences at least 80% identical thereto.

In various versions of the invention, the oligonucleotides in the arrays can be configured to bind at least a portion of a bisulfite modified sequence of any 1 or more, any 2 or more, any 3 or more, any 4 or more, any 5 or more, any 6 or more, any 7 or more, any 8 or more, any 9 or more, 10 or more, 11 or more, any 12 or more, any 13 or more, any 14 or more, any 15 or more, any 16 or more, any 17 or more, any 18 or more, any 19 or more, or each of SEQ ID NOS:1-20 or sequences at least 80% identical thereto.

In various versions of the invention, the above-referenced bisulfite modified sequences can comprise bisulfite modified sequences of any 1 or more, any 2 or more, any 3 or more, any 4 or more, any 5 or more, any 6 or more, any 7 or more, any 8 or more, any 9 or more, 10 or more, 11 or more, any 12 or more, any 13 or more, any 14 or more, or each of SEQ ID NOS:1-15 or sequences at least 80% identical thereto.

In various versions of the invention, the above-referenced bisulfite modified sequences can comprise bisulfite modified sequences of any 1 or more, any 2 or more, any 3 or more, any 4 or more, any 5 or more, any 6 or more, any 7 or more, any 8 or more, any 9 or more, or each of SEQ ID NOS:1-10 or sequences at least 80% identical thereto.

In various versions of the invention, the above-referenced bisulfite modified sequences can comprise bisulfite modified sequences of any 1 or more, any 2 or more, any 3 or more, any 4 or more, or each of SEQ ID NOS:1-5 or sequences at least 80% identical thereto.

In various versions of the invention, the above-referenced bisulfite modified sequences can comprise bisulfite modified sequences of any 1 or more, any 2 or more, or each of SEQ ID NOS:1-3 or sequences at least 80% identical thereto.

The sequences at least 80% identical to the aforementioned SEQ ID NOS can be at least 85%, at least 90%, at least 95%, at least 99%, or 100% identical thereto.

The portion of the sequences to which the oligonucleotides in the arrays can be configured to bind can include bisulfite modified forms of at least 5, at least 10, at least 15, at least 20, at least 25, at least 30, at least 35, at least 40, at least 45, or at least 50 contiguous bases in SEQ ID NOS:1-377, or any complement thereof. Exemplary oligonucleotides for including in the arrays of the invention for binding bisulfite modified forms of SEQ ID NOS:1-377 for determining the methylation status of one or more CpGs therein include SEQ ID NOS:378-754, respectively. “Binding” in this context does not require 100% base-by-base complementarity along the contiguous string of bases, as some internal mismatches, such as up to 20% by number, up to 15% by number, up to 10% by number, up to 5% by number, or up to 1% by number, can be permitted for the oligonucleotides to bind the defined contiguous string of bases overall. The outer binding bases will determine the length of the contiguous string of bases that are considered to be bound. In addition, any description of binding or of determining a methylation status of any sequence disclosed herein can refer to the binding or determination of the methylation status of the sequence's complement, as the context permits. The disclosure of determining the methylation status of a CpG site in a given sequence can refer to determining the methylation of a CpG in the sequence or its complement.

In some embodiments, the oligonucleotides in the arrays of the invention can be configured to bind amplicons comprising no more than 800,000 different CpG sites, no more than 700,000 different CpG sites, no more than 600,000 different CpG sites, no more than 500,000 different CpG sites, no more than 400,000 different CpG sites, no more than 300,000 different CpG sites, no more than 250,000 different CpG sites, no more than 200,000 different CpG sites, no more than 150,000 different CpG sites, no more than 100,000 different CpG sites, no more than 75,000 different CpG sites, no more than 50,000 different CpG sites, no more than 30,000 different CpG sites, no more than 25,000 different CpG sites, no more than 10,000 different CpG sites, no more than 5,000 different CpG sites, no more than 2,500 different CpG sites, no more than 1,000 different CpG sites, no more than 750 different CpG sites, no more than 500 different CpG sites, no more than 400 different CpG sites, no more than 300 different CpG sites, no more than 200 different CpG sites, no more than 100 different CpG sites, no more than 75 different CpG sites, no more than 50 different CpG sites, or no more than 25 different CpG sites. The CpG sites in such embodiments can comprise or consist in bisulfite modified CpG cites. A CpG site is considered to be “different” in this context if it has or corresponds to a different genomic position.

In some embodiments, the oligonucleotides in the arrays of the invention can be configured to bind amplicons of no more than 800,000 separate genomic regions, no more than 700,000 separate genomic regions, no more than 600,000 separate genomic regions, no more than 500,000 separate genomic regions, no more than 400,000 separate genomic regions, no more than 300,000 separate genomic regions, no more than 250,000 separate genomic regions, no more than 200,000 separate genomic regions, no more than 150,000 separate genomic regions, no more than 100,000 separate genomic regions, no more than 75,000 separate genomic regions, no more than 50,000 separate genomic regions, no more than 30,000 separate genomic regions, no more than 25,000 separate genomic regions, no more than 10,000 separate genomic regions, no more than 5,000 separate genomic regions, no more than 2,500 separate genomic regions, no more than 1,000 separate genomic regions, no more than 750 separate genomic regions, no more than 500 separate genomic regions, no more than 400 separate genomic regions, no more than 300 separate genomic regions, no more than 200 separate genomic regions, no more than 100 separate genomic regions, no more than 75 separate genomic regions, no more than 50 separate genomic regions, or no more than 25 separate genomic regions in the genome of the subject. The genomic regions in this context can comprise or consist of regions of bisulfite modified genomic DNA. Genomic regions are considered be “separate” in this context when they comprise at least one base separating each other in the original genome.

In some embodiments, the array comprises no more than 800,000 different oligonucleotides, no more than 700,000 different oligonucleotides, no more than 600,000 different oligonucleotides, no more than 500,000 different oligonucleotides, no more than 400,000 different oligonucleotides, no more than 300,000 different oligonucleotides, no more than 250,000 different oligonucleotides, no more than 200,000 different oligonucleotides, no more than 150,000 different oligonucleotides, no more than 100,000 different oligonucleotides, no more than 75,000 different oligonucleotides, no more than 50,000 different oligonucleotides, no more than 30,000 different oligonucleotides, no more than 25,000 different oligonucleotides, no more than 10,000 different oligonucleotides, no more than 5,000 different oligonucleotides, no more than 2,500 different oligonucleotides, no more than 1,000 different oligonucleotides, no more than 750 different oligonucleotides, no more than 500 different oligonucleotides, no more than 400 different oligonucleotides, no more than 300 different oligonucleotides, no more than 200 different oligonucleotides, no more than 100 different oligonucleotides, no more than 75 different oligonucleotides, no more than 50 different oligonucleotides, or no more than 25 different oligonucleotides. Oligonucleotides in this context are considered be “different” when they have a different sequence.

Methylated DNA has been studied as a potential class of biomarkers in a number of conditions. In many instances, DNA methyltransferases add a methyl group to DNA at cytosine-phosphate-guanine (CpG) island sites as an epigenetic control of gene expression. DNA methylation may be a more chemically and biologically stable diagnostic tool than RNA or protein expression (Laird (2010) Nat Rev Genet 11: 191-203).

Analysis of CpG islands has yielded important findings when applied to animal models and human cell lines. For example, Zhang and colleagues found that amplicons from different parts of the same CpG island may have different levels of methylation (Zhang et al. (2009) PLoS Genet 5: e1000438). Methylation levels were distributed bi-modally between highly methylated and unmethylated sequences, further supporting the binary switch-like pattern of DNA methyltransferase activity (Zhang et al. (2009) PLoS Genet 5: e1000438).

Several methods are available to search for novel methylation markers. There are three basic approaches. The first employs digestion of DNA by restriction enzymes which recognize specific methylated sites, followed by several possible analytic techniques which provide methylation data limited to the enzyme recognition site or the primers used to amplify the DNA in quantification steps (such as methylation-specific PCR; MSP). A second approach enriches methylated fractions of genomic DNA using anti-bodies directed to methyl-cytosine or other methylation-specific binding domains followed by microarray analysis or sequencing to map the fragment to a reference genome. This approach does not provide single nucleotide resolution of all methylated sites within the fragment. A third approach begins with bisulfite treatment of the DNA to convert all unmethylated cytosines to uracil, followed by various methylation assay procedures (e.g., microarray-based and sequencing analysis).

Provided herein is technology for methods for monitoring circannual rhythms by detecting DNA methylation.

As described herein, the technology provides a number of methylated DNA markers and subsets thereof (e.g., sets of 2, 3, 4, 5, 6, 7, 8, 9, 10, 11, 12, 13, 14, 15, 16, 17, 18, 19, 20, 21, or more markers) with high discrimination for monitoring circannual rhythms.

In some embodiments, the technology is related to assessing the presence of and methylation state of one or more of the markers identified herein in a biological sample (e.g., blood sample). These markers comprise one or more differentially methylated regions (DMRs) or differentially methylated positions (DMPs) in the genomic regions discussed herein, e.g., as provided in Table 2. Methylation state is assessed in embodiments of the technology. As such, the technology provided herein is not restricted in the method by which a gene's methylation state is measured. For example, in some embodiments the methylation state is measured by a genome scanning method. For example, one method involves restriction landmark genomic scanning (Kawai et al. (1994) Mol. Cell. Biol. 14: 7421-7427) and another example involves methylation-sensitive arbitrarily primed PCR (Gonzalgo et al. (1997) Cancer Res. 57: 594-599). In some embodiments, changes in methylation patterns at specific CpG sites are monitored by digestion of genomic DNA with methylation-sensitive restriction enzymes followed by Southern analysis of the regions of interest (digestion-Southern method). In some embodiments, analyzing changes in methylation patterns involves a PCR-based process that involves digestion of genomic DNA with methylation-sensitive restriction enzymes prior to PCR amplification (Singer-Sam et al. (1990) Nucl. Acids Res. 18: 687). In addition, other techniques have been reported that utilize bisulfite treatment of DNA as a starting point for methylation analysis. These include methylation-specific PCR (MSP) (Herman et al. (1992) Proc. Natl. Acad. Sci. USA 93: 9821-9826) and restriction enzyme digestion of PCR products amplified from bisulfite-converted DNA (Sadri and Hornsby (1996) Nucl. Acids Res. 24: 5058-5059; and Xiong and Laird (1997) Nucl. Acids Res. 25: 2532-2534). PCR techniques have been developed for detection of gene mutations (Kuppuswamy et al. (1991) Proc. Natl. Acad. Sci. USA 88: 1143-1147) and quantification of allelic-specific expression (Szabo and Mann (1995) Genes Dev. 9: 3097-3108; and Singer-Sam et al. (1992) PCR Methods Appl. 1: 160-163). Such techniques use internal primers, which anneal to a PCR-generated template and terminate immediately 5′ of the single nucleotide to be assayed. Methods using a “quantitative Ms-SNuPE assay” as described in U.S. Pat. No. 7,037,650 are used in some embodiments. Methylation arrays, such as the Infinium HD Methylation Assay (Pidsley et al. (2016) Genome Biol. 17:208), are used in some embodiments. In other embodiments, direct sequencing including next generation sequencing methods are used to assess the methylation status of subject DNA comprising, for example, Sanger sequencing (Sanger F et al (1977). DNA sequencing with chain-terminating inhibitors. Proc. Natl. Acad. Sci. U.S.A. 74 (12): 5463-7), Illumina NovSeq sequencing (Raine A et al (2018) Data quality of whole genome bisulfite sequencing on Illumina platforms. PLoS ONE 13(4): e0195972), PacBio sequencing (Eid J et al (2009) Real-time DNA sequencing from single polymerase molecules. Science. 323: 133-138), 454 sequencing (Margulies M et al (2005). Genome Sequencing in Open Microfabricated High Density Picoliter Reactors. Nature. 437(7057): 376-380), Ion Torrent sequencing (Rothberg J M et al (2011): An integrated semiconductor device enabling non-optical genome sequencing. Nature. 475 (7356): 348-352), and Oxford Nanopore sequencing (Eisenstein, M (2012). Oxford Nanopore announcement sets sequencing sector abuzz. Nat Biotechnol 30, 295-296), and other next generation sequencing platforms.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated and the resulting methylation-specific sequence is determined using either a real-time PCR or other exponential amplification, e.g., a QUARTS assay (e.g., as provided by U.S. Pat. No. 8,361,720; and U.S. Pat. Appl. Pub. Nos. 2012/0122088 and 2012/0122106, incorporated herein by reference).

For example, in some embodiments methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amount of methylated DNAs relative to the total amount of DNAs in the population, e.g., (number of methylated DNAs)/(the number of methylated DNAs+number of unmethylated DNAs)×100.

In some embodiments, the methods comprise determining a methylation level of any one or more CpG sites in one or more genomic regions of a genome of a subject. “Genomic region” as used herein can be defined by a sequence, a gene, and/or genomic coordinate(s), such as the sequences, genes, and/or genomic coordinates identified in Table 2. The genomic regions of the invention can accordingly comprise, consist essentially of, or consist of any one or more sequences, genes, genomic coordinates, or CpG sites identified in Table 2, any sequences, genes, genomic coordinates, or CpG sites aligning thereto, and/or any sequence at least 80%, 85%, 90%, 95%, 96%, 96%, 98%, or 99% identical to any sequence identified in Table 2, in any combination.

In some embodiments, the determining comprises: treating genomic DNA from the subject with bisulfite to generate bisulfite-treated genomic DNA; amplifying the bisulfite-treated genomic DNA using primers specific for a portion of the bisulfite-treated genomic DNA comprising the one or more genomic regions; and measuring the methylation level of the one or more CpG sites in the one or more genomic regions. The phrase “primers specific for a portion” of a given nucleic acid means that the primer is configured (has a sequence and length) to specifically hybridize within that portion of the nucleic acid (e.g., DNA) to specifically amplify that portion of DNA. “Specifically hybridize” and “specifically amplify” means that the primers bind to and amplify substantially only that portion of DNA (with “off-target amplification being only within the acceptable standards of the art). The specific primers are distinct from, for example, primers that hybridize and efficiently amplify multiple distinct targets across the genome, such as occurs with multi-target primers used for whole-genome amplification. “Portion” as used herein with reference to a given nucleic acid (e.g., DNA) refers to a region of the nucleic acid. “Bisulfite-modified forms” of the one or more genomic regions will have sequences identical to the original sequences of the genomic regions except for modifications of unmethylated cytosines to uracils, which are subsequently converted to thymines during PCR.

Some embodiments further comprise isolating the genomic DNA from the subject or a biological sample from the subject. In some embodiments, the biological sample is a blood sample. In some embodiments, the portion of the bisulfite-treated genomic DNA has a length less than 1000 bases, less than 950 bases, less than 900 bases, less than 850 bases, less than 800 bases, less than 750 bases, less than 700 bases, less than 650 bases, less than 600 bases, less than 550 bases, less than 500 bases, less than 450 bases, less than 400 bases, 350 bases, less than 300 bases, less than 250 bases, less than 200 bases, less than 150 bases, less than 100 bases, less than 75 bases, or less than 50 bases. In some embodiments, an amplicon resulting from the amplification has a length less than 1000 bases, less than 950 bases, less than 900 bases, less than 850 bases, less than 800 bases, less than 750 bases, less than 700 bases, less than 650 bases, less than 600 bases, less than 550 bases, less than 500 bases, less than 450 bases, less than 400 bases, 350 bases, less than 300 bases, less than 250 bases, less than 200 bases, less than 150 bases, less than 100 bases, less than 75 bases, or less than 50 bases. In some embodiments, an amplicon resulting from the amplification has a length of at least 100 bases, at least 150 bases, or at least 200 bases and less than 400 bases, less than 350 bases or less than 300 bases. In some versions, the primers can comprise sequencing adapters, such as next-generation sequencing adapters.

In some embodiments, the methylation level is measured by methylation-specific PCR, quantitative methylation-specific PCR, methylation-sensitive DNA restriction enzyme analysis, or bisulfite genomic sequencing PCR, or quantitative bisulfite pyrosequencing.

In some embodiments, the subject is a mammal. In some embodiments, the subject is a cat, a bovine, a dog, a dolphin, an elephant, a horse, a human, or a mouse. In some embodiments, the subject is a mouse. In some embodiments, the subject is a human.

Also provided herein are compositions and kits and systems for practicing the methods of the invention. For example, in some embodiments, reagents (e.g., primers, probes) specific for one or more markers are provided alone or in sets (e.g., sets of primers pairs for amplifying a plurality of markers). Additional reagents for conducting a detection assay may also be provided (e.g., enzymes, buffers, positive and negative internal and external controls for conducting QuARTS, PCR, sequencing, bisulfite, calibrants or other assays). In some embodiments, the kits containing one or more reagents necessary, sufficient, or useful for conducting a method are provided. Also provided are reactions mixtures containing the reagents, and instructions for use of the reagents. Further provided are master mix reagent sets containing a plurality of reagents that may be added to each other and/or to a test sample to complete a reaction mixture.

Some embodiments provide methods comprising assaying a plurality of markers. In some embodiments, the plurality of markers comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 110, at least 120, at least 130, at least 140, or at least 150 markers. In some embodiments, the plurality of markers comprise fewer than 3, fewer than 4, fewer than 5, fewer than 6, fewer than 7, fewer than 8, fewer than 9, fewer than 10, fewer than 11, fewer than 12, fewer than 13, fewer than 14, fewer than 15, fewer than 16, fewer than 17, fewer than 18, fewer than 19, fewer than 20 fewer than 30, fewer than 40, fewer than 50, fewer than 60, fewer than 70, fewer than 80, fewer than 90, fewer than 100, fewer than 110, fewer than 120, fewer than 130, fewer than 140, or fewer than 150 markers.

The technology is not limited in the methylation state assessed. In some embodiments assessing the methylation state of the marker in the sample comprises determining the methylation state of one base. In some embodiments, assaying the methylation state of the marker in the sample comprises determining the extent of methylation at a plurality of bases. Moreover, in some embodiments the methylation state of the marker comprises an increased methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

Furthermore, in some embodiments the marker is a region of 100 or fewer bases, the marker is a region of 500 or fewer bases, the marker is a region of 1000 or fewer bases, the marker is a region of 5000 or fewer bases, or, in some embodiments, the marker is one base.

The technology is not limited by sample type. For example, in some embodiments the sample is a blood sample (e.g., plasma, serum, whole blood), a stool sample, a tissue sample (e.g., lung tissue sample), an excretion, or a urine sample.

Furthermore, the technology is not limited in the method used to determine methylation state. In some embodiments the assaying comprises using methylation specific polymerase chain reaction, nucleic acid sequencing, mass spectrometry, methylation specific nuclease, mass-based separation, or target capture. In some embodiments, the assaying comprises use of a methylation specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc. In some embodiments, the technology uses array-based methylation analysis.

The technology provides reagents for detecting the methylation state of a DMR and/or DMP, e.g., in some embodiments are provided a set of oligonucleotides comprising the sequences of the oligonucleotides provided herein. In some embodiments are provided an oligonucleotide comprising a sequence complementary to a chromosomal region having a base in a DMR, e.g., an oligonucleotide sensitive to methylation state of a DMR and/or DMP. The oligonucleotides can be immobilized on a sold surface in an array.

Kit embodiments are provided, e.g., a kit comprising a bisulfite reagent; and a control nucleic acid comprising a sequence from a genomic region of the invention and having a methylation state associated with a subject who has or does not have a condition as described herein. In some embodiments, kits comprise a bisulfite reagent and an oligonucleotide as described herein. In some embodiments, kits comprise a bisulfite reagent; and a control nucleic acid comprising a sequence from a genomic region of the invention and having a methylation state as described in the following examples. Some kit embodiments comprise a sample collector for obtaining a sample from a subject (e.g., blood sample); reagents for isolating a nucleic acid from the sample; a bisulfite reagent; and an oligonucleotide as described herein.

The technology is related to embodiments of compositions (e.g., reaction mixtures). In some embodiments are provided a composition comprising a nucleic acid comprising a DMR and/or a DMP and a bisulfite reagent. Some embodiments provide a composition comprising a nucleic acid comprising a DMR and/or a DMP and an oligonucleotide as described herein. Some embodiments provide a composition comprising a nucleic acid comprising a DMR and/or a DMP a methylation-sensitive restriction enzyme. Some embodiments provide a composition comprising a nucleic acid comprising a DMR and/or a DMP and a polymerase.

In certain embodiments, methods for characterizing a sample (e.g., blood sample) from a subject are provided. For example, some embodiments comprise obtaining DNA from a sample of a subject; assaying a methylation state of a DNA methylation marker comprising a base in a genomic region of the invention; and comparing the assayed methylation state of the one or more DNA methylation markers with negative and/or positive methylation level references for the one or more DNA methylation markers.

Such methods are not limited to a particular type of sample from a subject. In some embodiments, the sample is a blood sample. In some embodiments, the sample is a stool sample, a tissue sample, or a urine sample.

In some embodiments, the DNA methylation marker is a region of 100 or fewer bases. In some embodiments, the DNA methylation marker is a region of 500 or fewer bases. In some embodiments, the DNA methylation marker is a region of 1000 or fewer bases. In some embodiments, the DNA methylation marker is a region of 5000 or fewer bases. In some embodiments, the DNA methylation marker is one base.

In some embodiments, the assaying comprises using methylation specific polymerase chain reaction, nucleic acid sequencing, mass spectrometry, methylation specific nuclease, mass-based separation, or target capture.

In some embodiments, the assaying comprises use of a methylation specific oligonucleotide.

In certain embodiments, the technology provides methods for characterizing a sample obtained from a subject. In some embodiments, such methods comprise determining a methylation state of one or more DNA methylation markers in the sample comprising one or more bases in one or more genomic regions of the invention; comparing the methylation state of the one or more DNA methylation markers from the subject sample to a methylation state of the one or more DNA methylation markers from a control sample from a control subject; and determining a confidence interval and/or a p value of the difference in the methylation state of subject sample and the control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001.

In certain embodiments, the technology provides methods for characterizing a sample obtained from a subject (e.g., blood sample), the method comprising reacting a nucleic acid comprising at least one DMR with a bisulfite reagent to produce a bisulfite-reacted nucleic acid; sequencing the bisulfite-reacted nucleic acid to provide a nucleotide sequence of the bisulfite-reacted nucleic acid; comparing the nucleotide sequence of the bisulfite-reacted nucleic acid with a nucleotide sequence of a nucleic acid comprising the DMR from a control subject.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The human genomic positions described herein refer to genomic positions as provided in the UCSC hg19 human reference genome (Fujita P A, Rhead B, Zweig A S, Hinrichs A S, Karolchik D, Cline M S, Goldman M, Barber G P, Clawson H, Coelho A, Diekhans M, Dreszer T R, Giardine B M, Harte R A, Hillman-Jackson J, Hsu F, Kirkup V, Kuhn R M, Learned K, Li C H, Meyer L R, Pohl A, Raney B J, Rosenbloom K R, Smith K E, Haussler D, Kent W J. The UCSC Genome Browser database: update 2011. Nucleic Acids Res. 2011 January; 39(Database issue):D876-82. doi: 10.1093/nar/gkg963.) and positions in other genomes aligning thereto. Suitable alignment methods are known in the art. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The genomic positions of any CpG sites described herein refer to the position of the C in the CpG site in the original genome.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

“Amplification” is a special case of nucleic acid replication involving template specificity. It is to be contrasted with non-specific template replication (e.g., replication that is template-dependent but not dependent on a specific template). Template specificity is here distinguished from fidelity of replication (e.g., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Amplification of nucleic acids generally refers to the production of multiple copies of a polynucleotide, or a portion of the polynucleotide, typically starting from a small amount of the polynucleotide (e.g., a single polynucleotide molecule, 10 to 100 copies of a polynucleotide molecule, which may or may not be exactly the same), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), Hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.”

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-beta replicase, MDV-1 RNA is the specific template for the replicase (Kacian et al., Proc. Natl. Acad. Sci. USA, 69:3038 [1972]). Other nucleic acid will not be replicated by this amplification enzyme. Similarly, in the case of T7 RNA polymerase, this amplification enzyme has a stringent specificity for its own promoters (Chamberlin et al, Nature, 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics 4:560). Finally, thermostable template-dependent DNA polymerases (e.g., Taq and Pfu DNA polymerases), by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and US 2009/0253142, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Barnay Proc. Natl. Acad. Sci USA 88, 189-93 (1991)); and sandwich hybridization methods (e.g., U.S. Pat. No. 5,288,609, herein incorporated by reference in its entirety).

The term “amplifiable nucleic acid” refers to a nucleic acid that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as a DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated. With respect to a single CpG locus at a single pair of sister chromosomes in a single cell, a CpG may be 100% methylated (both Cs are methylated, 50% methylated (one C is methylated and the other is not), or 0% methylated (neither C of the paired sister chromosomes is methylated at the specific CpG locus). Accordingly, methylation of all sister chromosomes in a population of cells at a specific CpG may be 0-100% methylated depending on the relative proportion of cells that are 100%, 50% or 0% methylated at a specific CpG locus in a mixture of cells in a sample.

As such, the methylation state describes the state of methylation of a nucleic acid (e.g., a genomic region). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between subjects that have recurrence of cancer after surgery versus subjects who not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such a change in the nucleic acid molecule's nucleotide sequence can result in a nucleic acid molecule in which each methylated nucleotide is modified to a different nucleotide. Such a change in the nucleic acid nucleotide sequence can result in a nucleic acid molecule in which each unmethylated nucleotide is modified to a different nucleotide. Such a change in the nucleic acid nucleotide sequence can result in a nucleic acid molecule in which each of a selected nucleotide which is unmethylated (e.g., each unmethylated cytosine) is modified to a different nucleotide. Use of such a reagent to change the nucleic acid nucleotide sequence can result in a nucleic acid molecule in which each nucleotide that is a methylated nucleotide (e.g., each methylated cytosine) is modified to a different nucleotide. As used herein, use of a reagent that modifies a selected nucleotide refers to a reagent that modifies one nucleotide of the four typically occurring nucleotides in a nucleic acid molecule (C, G, T, and A for DNA and C, G, U, and A for RNA), such that the reagent modifies the one nucleotide without modifying the other three nucleotides. In one exemplary embodiment, such a reagent modifies an unmethylated selected nucleotide to produce a different nucleotide. In another exemplary embodiment, such a reagent can deaminate unmethylated cytosine nucleotides. An exemplary reagent is bisulfite.

As used herein, the term “bisulfite reagent” refers to a reagent comprising in some embodiments bisulfite, disulfite, hydrogen sulfite, or combinations thereof to distinguish between methylated and unmethylated cytidines, e.g., in CpG dinucleotide sequences.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al. (1997) Cancer Research 57: 594-599.

The term “MethyLight™” refers to the art-recognized fluorescence-based real-time PCR technique described by Eads et al. (1999) Cancer Res. 59: 2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25: 2529-2531.

The term “MSP” (Methylation-specific PCR) refers to the art-recognized methylation assay described by Herman et al. (1996) Proc. Natl. Acad. Sci. USA 93: 9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25: 2532-2534.

The term “MCA” (Methylated CpG Island Amplification) refers to the methylation assay described by Toyota et al. (1999) Cancer Res. 59: 2307-12, and in WO 00/26401A1.

The term “Infinium HD Methylation Assay” refers to the methylation assay described by Pidsley et al. (2016) Genome Biol. 17:208.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The terms “methylation-specific restriction enzyme” or “methylation-sensitive restriction enzyme” refers to an enzyme that selectively digests a nucleic acid dependent on the methylation state of its recognition site. In the case of a restriction enzyme that specifically cuts if the recognition site is not methylated or is hemimethylated, the cut will not take place or will take place with a significantly reduced efficiency if the recognition site is methylated. In the case of a restriction enzyme that specifically cuts if the recognition site is methylated, the cut will not take place or will take place with a significantly reduced efficiency if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, a “different nucleotide” refers to a nucleotide that is chemically different from a selected nucleotide, typically such that the different nucleotide has Watson-Crick base-pairing properties that differ from the selected nucleotide, whereby the typically occurring nucleotide that is complementary to the selected nucleotide is not the same as the typically occurring nucleotide that is complementary to the different nucleotide. For example, when C is the selected nucleotide, U or T can be the different nucleotide, which is exemplified by the complementarity of C to G and the complementarity of U or T to A. As used herein, a nucleotide that is complementary to the selected nucleotide or that is complementary to the different nucleotide refers to a nucleotide that base-pairs, under high stringency conditions, with the selected nucleotide or different nucleotide with higher affinity than the complementary nucleotide's base-paring with three of the four typically occurring nucleotides. An example of complementarity is Watson-Crick base pairing in DNA (e.g., A-T and C-G) and RNA (e.g., A-U and C-G). Thus, for example, G base-pairs, under high stringency conditions, with higher affinity to C than G base-pairs to G, A, or T and, therefore, when C is the selected nucleotide, G is a nucleotide complementary to the selected nucleotide.

The term “marker”, as used herein, refers to a substance (e.g., a nucleic acid or a region of a nucleic acid) that is able to indicate the presence of a condition, e.g., based its methylation state.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, by be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it is meant to include a specimen or culture obtained from any source, as well as biological and environmental samples. Biological samples may be obtained from plants or animals (including humans) and encompass fluids, solids, tissues, and gases. In some embodiments, the sample is a blood sample. Environmental samples include environmental material such as surface matter, soil, water, and industrial samples. These examples are not to be construed as limiting the sample types applicable to the present invention.

As used herein, the terms “subject” refer to organisms to be subject to various tests provided by the technology. The term “subject” includes animals, preferably mammals, including humans and mice.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

In particular aspects, the present technology provides compositions and methods for monitoring circannual rhythms, such as determining birth month or predicting breeding behavior. The methods comprise determining the methylation status of at least one methylation marker in a biological sample isolated from a subject (e.g., blood sample), wherein a change in the methylation state of the marker is indicative of one or more aspects of a circannual rhythm. Particular embodiments relate to markers comprising one or more DMRs and/or DMPs in the genomic regions identified in Table2.

Some embodiments of the technology are based upon the analysis of the CpG methylation status of at least one marker, region of a marker, or base of a marker comprising one or more DMRs and/or DMPs in the genomic regions identified in Table 2.

In some embodiments, the present technology provides for the use of the bisulfite technique in combination with one or more methylation assays to determine the methylation status of CpG dinucleotide sequences within at least one marker comprising one or more DMRs and/or DMPs in the genomic regions identified in Table 2. Genomic CpG dinucleotides can be methylated or unmethylated (alternatively known as up- and down-methylated respectively). However, the methods of the present invention are suitable for the analysis of biological samples of a heterogeneous nature. Accordingly, when analyzing the methylation status of a CpG position within such a sample one may use a quantitative assay for determining the level (e.g., percent, fraction, ratio, proportion, or degree) of methylation at a particular CpG position.

In some embodiments, the technology relates to assessing the methylation state of combinations of markers comprising more than one DMR and/or DMP in one or more of the genomic regions identified in Table 2. In some embodiments, assessing the methylation state of more than one marker increases the specificity and/or sensitivity of detecting particular aspects of or in a subject, such as birth period.

The most frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil forms base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98) or methylation-specific PCR (MSP) as is disclosed, e.g., in U.S. Pat. No. 5,786,146.

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then either assaying the product by sequencing (Olek & Walter (1997) Nat. Genet. 17: 275-6) or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures are known in the art and can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing or microarray of bisulfite-treated nucleic acid, PCR (for sequence-specific amplification), Southern blot analysis, and use of methylation-sensitive restriction enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

In some embodiments, assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, DMR, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase.

MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, DMR, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, DMR, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can by used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, DMR, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, DMR, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QUARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes cleavase to release the flap sequence by cutting between the detection probe and the flap sequence. The flap sequence is complementary to a nonhairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophore per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199; U.S. patent application Ser. Nos. 12/946,737, 12/946,745, 12/946,752, and 61/548,639.

In some embodiments, an array such as the Infinium HD methylation assay is used to evaluate methylation state. After bisulfite treatment, the samples are denatured and neutralized to prepare them for amplification. The denatured DNA is isothermally amplified in an overnight step. The whole-genome amplification uniformly increases the amount of the DNA sample by several thousand-folds without significant amplification bias. A controlled enzymatic process fragments the amplified product. The process uses endpoint fragmentation to prevent overfragmentation. After precipitation and resuspension, the fragmented DNA is dispensed onto BeadChips. The BeadChips are incubated in the Illumina Hybridization Oven to hybridize the samples onto the BeadChips. Twelve samples are applied to each BeadChip, which keeps them separate with an IntelliHyb seal. The prepared BeadChip is incubated overnight in the Illumina Hybridization Oven. The amplified and fragmented DNA samples anneal to locus-specific 50mers (covalently linked to 1 of over 500,000 bead types) during hybridization. Two bead types correspond to each CpG locus for Infinium I assays: one bead type corresponds to methylated (C), another bead type to unmethylated (T) state of the CpG site. One bead type corresponds to each CpG locus for Infinium II assays. Then the unhybridized and nonspecifically hybridized DNA is washed away and the BeadChip is prepared for staining and extension in capillary flow-through chambers. Single-base extension of the oligos occurs on the BeadChip, using the captured DNA as a template, which incorporates detectable labels on the BeadChip and determines the methylation level of the query CpG sites. The Illumina HiScan or iScan System scans the BeadChip, using a laser to excite the fluorophore of the single-base extension product on the beads. The scanner records high-resolution images of the light emitted from the fluorophores.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715, which is incorporated by reference in its entirety). It is preferred that the bisulfite treatment is conducted in the presence of denaturing solvents such as but not limited to n-alkylenglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). The bisulfite conversion is preferably carried out at a reaction temperature between 30° C. and 70° C., whereby the temperature is increased to over 85° C. for short times during the reaction (see: PCT/EP2004/011715, which is incorporated by reference in its entirety). The bisulfite treated DNA is preferably purified prior to the quantification. This may be conducted by any means known in the art, such as but not limited to ultrafiltration, e.g., by means of Microcon™ columns (manufactured by Millipore™). The purification is carried out according to a modified manufacturer's protocol (see, e.g., PCT/EP2004/011715, which is incorporated by reference in its entirety).

In some embodiments, fragments of the treated DNA are amplified using sets of primer oligonucleotides according to the present invention and an amplification enzyme. The amplification of several DNA segments can be carried out simultaneously in one and the same reaction vessel. Typically, the amplification is carried out using a polymerase chain reaction (PCR). Amplicons are typically 100 to 2000 base pairs in length.

In another embodiment of the method, the methylation status of CpG positions within or near a marker comprising any one or more DMRs and/or DMPs in Table 2 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

Genomic DNA may be isolated by any means, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types are suitable for use in the present method, e.g., cell lines, histological slides, biopsies, paraffin-embedded tissue, body fluids, stool, prostate tissue, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a blood sample using direct gene capture.

The genomic DNA sample is then treated with at least one reagent, or series of reagents, that distinguishes between methylated and non-methylated CpG dinucleotides within at least one marker comprising one or more DMRs and/or DMPs in Table 2.

In some embodiments, the reagent converts cytosine bases which are unmethylated at the 5′-position to uracil, thymine, or another base which is dissimilar to cytosine in terms of hybridization behavior. However in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one genomic region, gene, genomic sequence, or nucleotide from a marker comprising one or more DMRs and/or DMPs in Table 2). The method of analysis may be selected from those known in the art, including those listed herein, e.g., QuARTS and MSP as described herein.

Aberrant methylation of a marker comprising one or more DMRs and/or DMPs in Table 2 as shown in the examples is associated with one or more conditions or states described herein, such as being born in a certain birth period.

In some embodiments, the method comprises providing a series of biological samples over a longitudinal or serial time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict or monitor an aspect of circannual rhythm.

As noted, in some embodiments, multiple determinations of one or more diagnostic or prognostic biomarkers can be made, and a temporal change in the marker can be used to determine a determination or prognosis. For example, a diagnostic marker can be determined at an initial time, and again at a second time. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR and/or DMP). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMRs and/or DMPs), the chance of a given outcome (e.g., suffering from severe clinical outcomes) may be very low.

In some embodiments, a statistical analysis associates a prognostic indicator with a predisposition to an adverse outcome. For example, in some embodiments, a methylation state different from that in a normal control sample obtained from a subject who does not have a particular adverse outcome can signal that a subject is more likely to suffer from a particular adverse outcome than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein (e.g., a DMR and/or DMP) can be established, and the degree of change in the methylation state of the biomarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal samples, as well as for “at-risk” levels of the one or more biomarkers in samples taken from donors with a particular condition. In certain embodiments of the method, a subject is identified as having a particular condition upon identifying an aberrant methylation state of one or more DMRs and/or DMPs provided herein in a biological sample obtained from the subject. In other embodiments of the method, the detection of an aberrant methylation state of one or more of such biomarkers in a biological sample obtained from the subject results in the subject being identified as having a particular condition.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is determined as having a particular condition if, when compared to a control methylation state, there is a measurable difference in the methylation state of at least one biomarker in the sample. Conversely, when no change in methylation state is identified in the biological sample, the subject can be identified as not having the condition, not being at risk for the condition, or as having a low risk of the condition. In this regard, subjects having the condition or risk thereof can be differentiated from subjects having low to substantially no condition or risk thereof. Those subjects having a risk of developing a particular condition can be placed on a more intensive and/or regular screening schedule or treatment. On the other hand, those subjects having low to substantially no risk may avoid being subjected to more intensive and/or regular screening schedule or treatment.

Depending on the embodiment of the method of the present technology, detecting a change in methylation state of the one or more biomarkers can be a qualitative determination or it can be a quantitative determination. As such, the step of diagnosing a subject as having, or at risk of developing a particular condition indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specifically state or range of state. As such, the predetermined methylation state can be chosen, within acceptable limits that will be apparent to those skilled in the art, based in part on the embodiment of the method being practiced and the desired specificity, etc.

The elements and method steps described herein can be used in any combination whether explicitly described or not, unless otherwise specified or clearly implied to the contrary by the context in which the referenced combination is made.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

As used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. The meaning of “in” includes “in” and “on.”

### Examples

### Circannual Breeding and Methylation are Impacted by the Equinox in Peromyscus

### Summary

Photoperiod is the major regulator of circannual patterns in mammals, but at animal facilities, despite the controlled conditions, some rodents still exhibit seasonality in their breeding. By evaluating more than 97,000 breeding records of closed Peromyscus colonies, we explored if seasonal breeding patterns exist in animals maintained for several decades at controlled environments.

Seasonal preferences for offspring production were recorded that depended on maternal birth month. Birth in March and September, especially of mothers, showed best fit in 12-knots generalized additive models, implying that the memory of birth month is more intensively carried during the Spring and Autumn equinoxes. The memory of birth month was associated with circannual CpG methylation at birth and revealed that most intense differential methylation, primarily demethylation, occurs in animals that were born in September. Comparison of breeding performance in the periods between the equinoxes showed preference for summer littering by the winter-born animals, especially in the polygamous P. maniculatus bairdii and P. leucopus stocks, and at least in the former species, this was associated with longer estrus cycles. Genes associated with differentially methylated CpGs in winter and summer periods in polygamous species pointed to regulation of gonadotropin hormone releasing hormone secretion. Birth at individually ventilated cages disrupted methylation profiles.

The cyclicity of breeding and methylation patterns suggest that signals other than the photoperiod regulate circannual patterns in deer mice, that reset in the equinox especially in autumn, and that the females are more sensitive to these signals than males.

Portions of the following examples are published in Huynh-Dam et al. [41], which is incorporated herein by reference in its entirety.

### Introduction

Circannual patterns are common in animals and reflect adaptations that warrant offspring production when food availability and the lack of predators are favorable for the growth of newborns [1, 2]. In humans, circannual patterns have been recorded in metabolic processes and endocrine circuits including those involving reproductive hormones, while season of birth has been linked to lifespan [3-7]. Photoperiod and temperature are the major factors that regulate circannual rhythms in nature; nonetheless, at controlled animal facilities, laboratory rodents also display evidence of seasonality, pointing to additional mechanisms regulating circannual rhythms [8, 9].

Deer mice in captivity (genus Peromyscus) may represent a suitable model to study seasonal rhythms because they are maintained at the Peromyscus Genetic Stock Center (PGSC) at controlled animal facilities for several decades as closed colonies of genetically diverse stocks. Breeding data are available since the original establishment of the colonies which enables analyses of reproductive patterns. In the present study, we explored the presence of seasonal patterns in the breeding of deer mice (Table 1). Our analyses involved about 97,000 entries, tracing back to 1962 for monogamous P. polionotus (PO stock) and polygamous P. maniculatus bairdii (BW stock), in 1988 for polygamous P. leucopus (LL stock), in 2002 for high-altitude P. maniculatus sonoriensis (SM2 stock), in 1989 for monogamous P. californicus (IS stock), and 1995 for monogamous P. eremicus (EP stock) [10]. Since then, the deer mice are maintained as closed colonies in the animal facilities of the PGSC. By analyzing the PGSC breeding records, we tested if the season of birth influences the season of littering in Peromyscus. Subsequently, we evaluated if such memory of birth season is reflected to the epigenome of deer mice in captivity.

TABLE 1
Number of breeding pairs, total offspring and birth events
for all the stocks used in the present analysis

| | Species (stocks) | | | | | |
|---|---|---|---|---|---|---|
| | BW | SM2 | LL | IS | PO | EP |
| | 1962- | 2002- | 1988- | 1989- | 1962- | 1995- |
| | 2023 | 2017 | 2023 | 2023 | 2023 | 2023 |
| Breeding pairs | 1,580 | 222 | 821 | 470 | 751 | 167 |
| Total offspring | 35,757 | 8,480 | 24,093 | 9,021 | 17,184 | 2,802 |
| Total births | 6,253 | 1,377 | 4,236 | 2,797 | 4,034 | 1,092 |

### Results

### Association Between Birth Season and Time of Littering

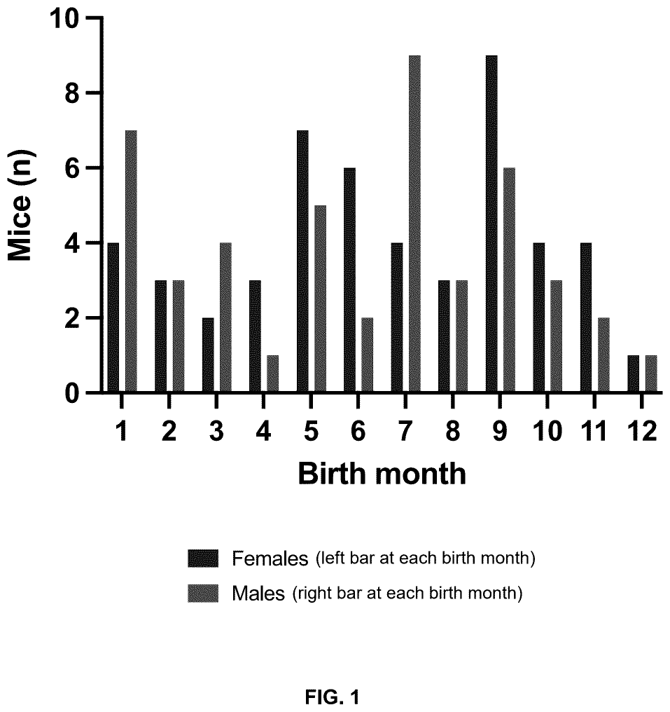

Initially, we evaluated if the birth month of the parents, and the month at which offspring are produced, are correlated. Our analysis indicated that offspring production does not occur randomly throughout the year and that birth at different months favors offspring production at particular periods, by a manner that exhibits periodic pattern. This preference was stronger for animals born in winter months, from September to February when offspring production was favored 6-7 months later, in the summer season, while for animals born in the summer, especially from May to July, offspring production abolished this preference and had occurred more randomly throughout the year (Huynh-Dam et al. [41] at Additional file 1: FIG. S1). This association between the parents' and the offspring's season of birth implies the operation of a circannual rhythm in breeding behavior which prompted us to explore if this correlation can be modelled mathematically. After testing other models for best fitness, including linear regression or polynomial regression, we concluded that both sinusoidal (SINE) model and 12-knots generalized additive model (GAM) accurately predict the reproductive behavior of the different Peromyscus stocks in captivity (Huynh-Dam et al. [41] at FIG. 1). The SINE model was selected for its ability to capture periodic patterns inherent in circannual rhythms, while the GAM offers the flexibility to account for complex, non-linear relationships between predictors and the response variable, which is vital given the multifactorial influences on reproductive physiology [11].

### The Impact of the Birth Season is Most Pronounced in Equinox

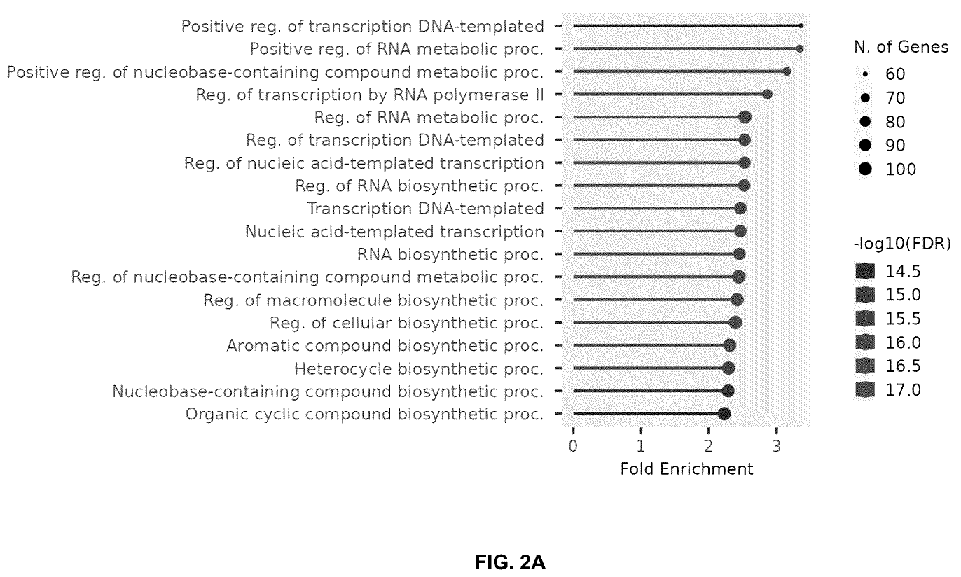

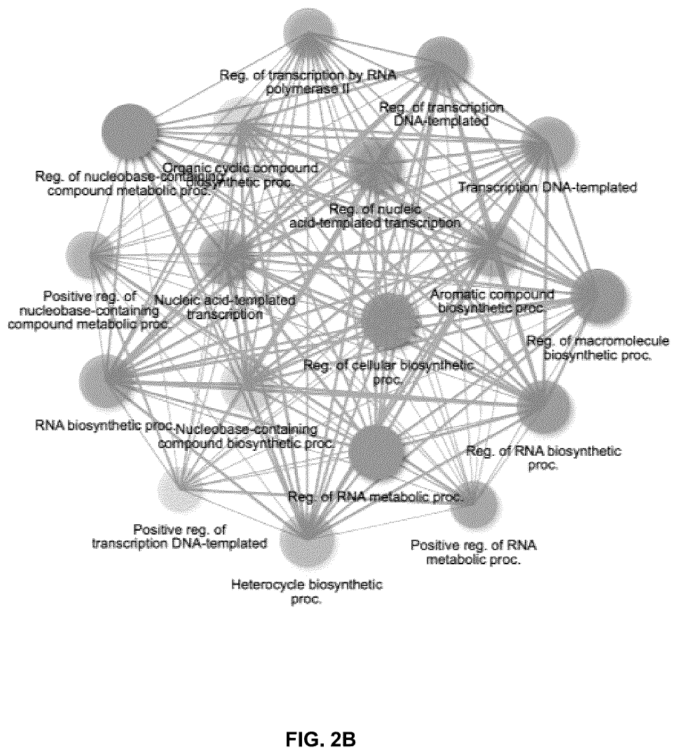

These observations suggest that birth season differentially impacts reproductive behavior in deer mice in captivity, which correlates the time of parents' birth with the time of littering. It is plausible that such effect will have differential strength at different times of the year, which in turn can be reflected to the magnitude of the correlation coefficient (R) of the corresponding SINE model or 12-knots GAM, for the different birth months, species, and sexes. This, in turn, may point to a specific time period that plays more prominent role in the imposition of such birth season memory and subsequently reflects to the animals' reproductive behavior. To test this hypothesis, we calculated the average coefficient of correlation (R) among the 6 stocks available, for both the sires and the dams, and for both the SINE and 12-knots GAM. We reasoned that since the breeding data used for this analysis have been normalized for the total number of offspring produced throughout the year, this rules out the possibility that our observations are contingent with the different times and the numbers of breeders set, the period at which they were maintained during the PGSC's operation, or the number of the offspring that differ among the stocks. Our analyses indicated that the average coefficient of correlation (R) among the 6 stocks available, for both the sires and the dams, exhibited good fitness in both the SINE and the 12-knots GAM, that followed a bimodal rhythm being considerably more robust in the female parents (dams) (Huynh-Dam et al. [41] at FIG. 2a). Fitness in 12-knots GAM was better, as indicated by the slightly higher R values, than that calculated by using the SINE model, but the trend was similar (Huynh-Dam et al. [41] at FIG. 2a). During the equinoxes, in March and September, R values were maximal for both models and were lower both before and after this time (Huynh-Dam et al. [41] at FIG. 2a). In sires, no considerable evidence of cyclicity was found with the exception of moderate peaks in R values in January and September (Huynh-Dam et al. [41] at FIG. 2a). Thus, we concluded that the equinoxes in March and September are of particular significance in the females, and determine the stringency of the reproductive pattern for animals born at subsequent periods.

### Circannual Rhythm in Peromyscus Breeding

The variation we recorded in the correlation between the breeding preference of the animals and their month of birth, which is tighter in March and September in the females, implies the operation of cyclicity in the stringency of this circannual clock. Indeed, the variation in the fitness (R values) of the reproduction data in both the 12-knots GAM (Huynh-Dam et al. [41] at FIGS. 2b and 2c) and the SINE model (Huynh-Dam et al. [41] at Additional file 1: FIG. S2) followed a periodic pattern that could be modeled by a simple sinusoidal (SINE) model (Huynh-Dam et al. [41] at FIG. 2d). P values are shown in Huynh-Dam et al. [41] at FIG. 2c, indicating higher significance in P. leucopus (LL), P. maniculatus bairdii (BW), and P. polionotus (PO) females. These three stocks are historically maintained at higher numbers which may contribute in providing more robust and coherent results. Our observations suggest that the females born around the equinox coordinate their breeding to produce litters at specific seasons. This coordination occurs for animals born at different seasons but is weaker and is maximized in March and September equinox.

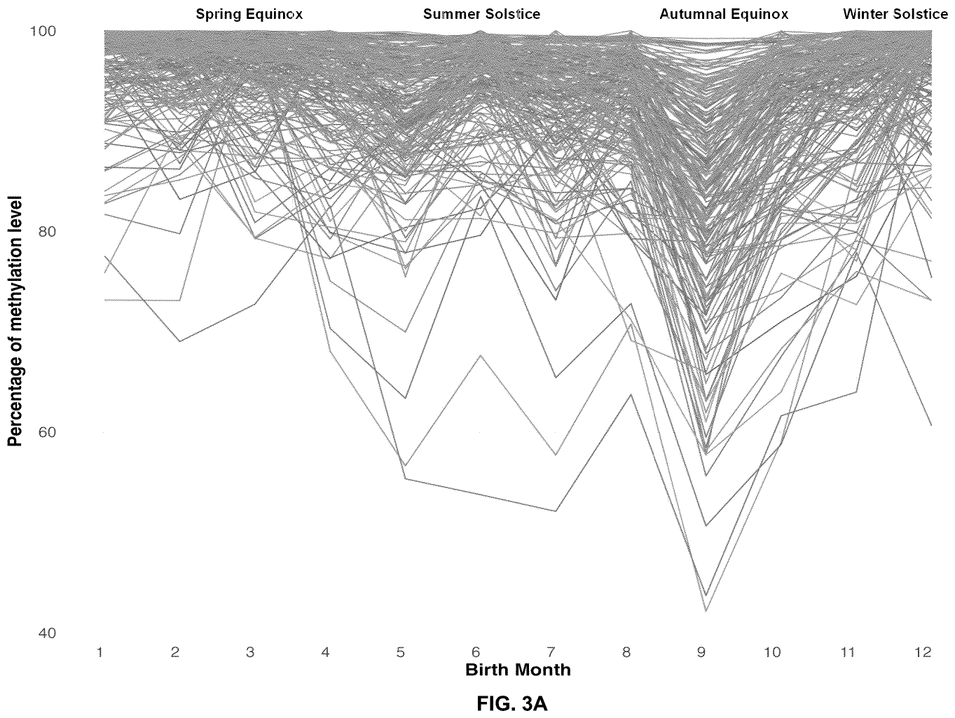

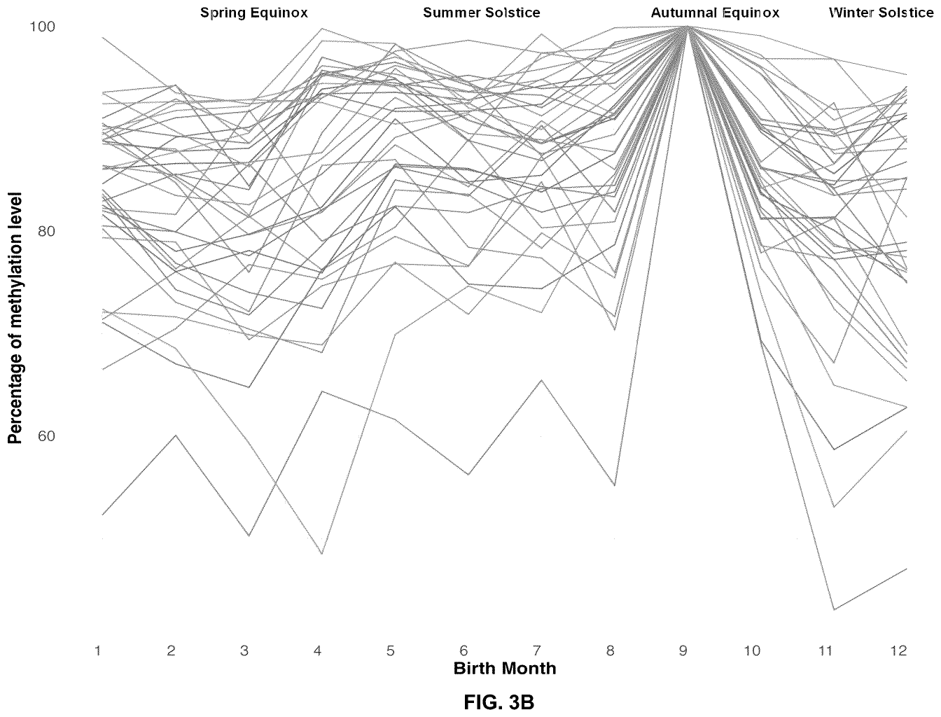

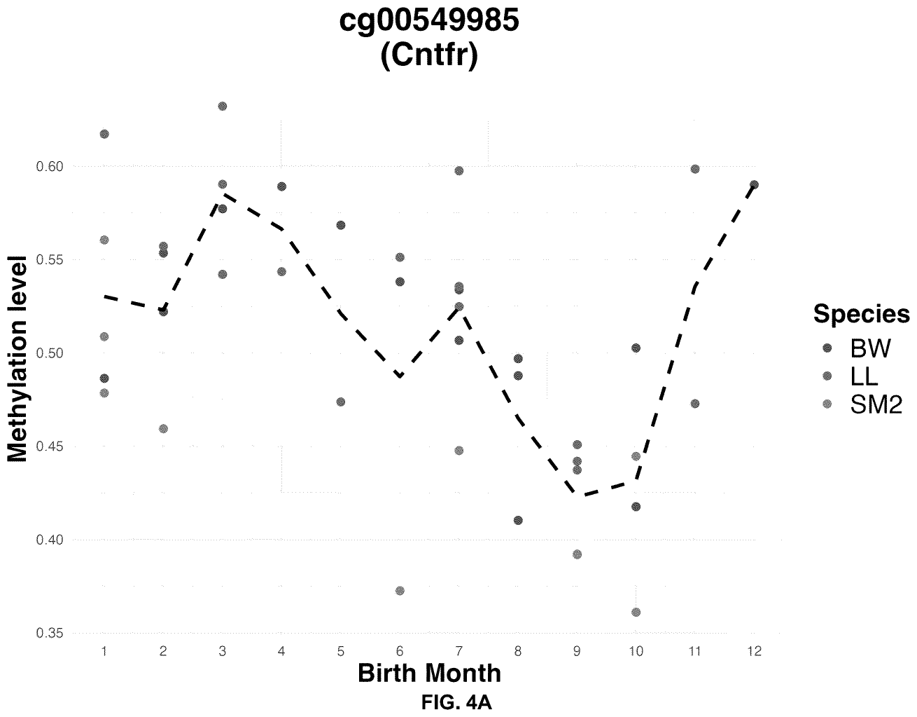

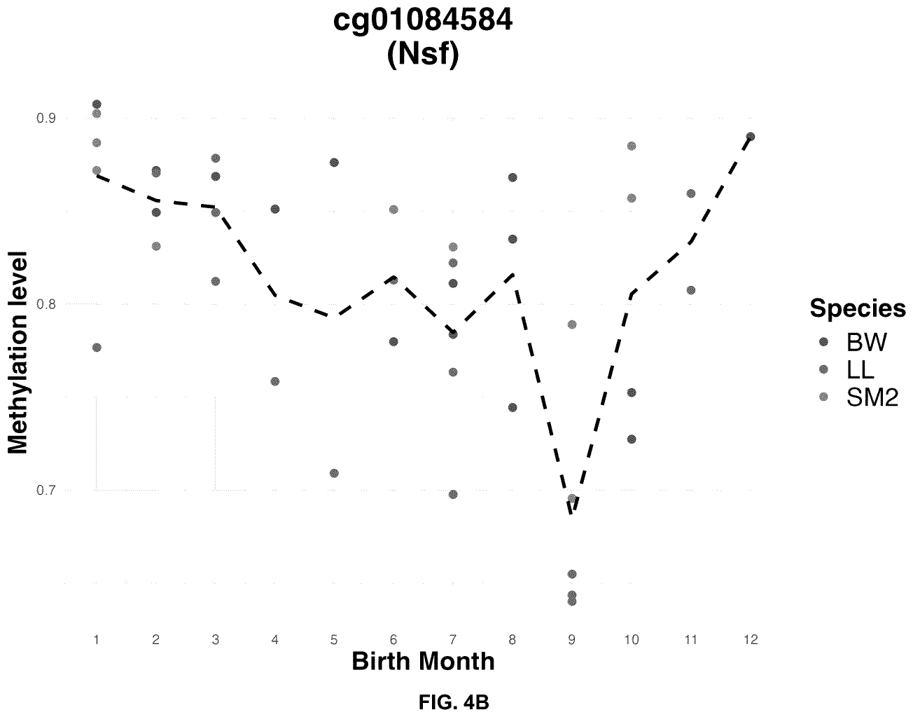

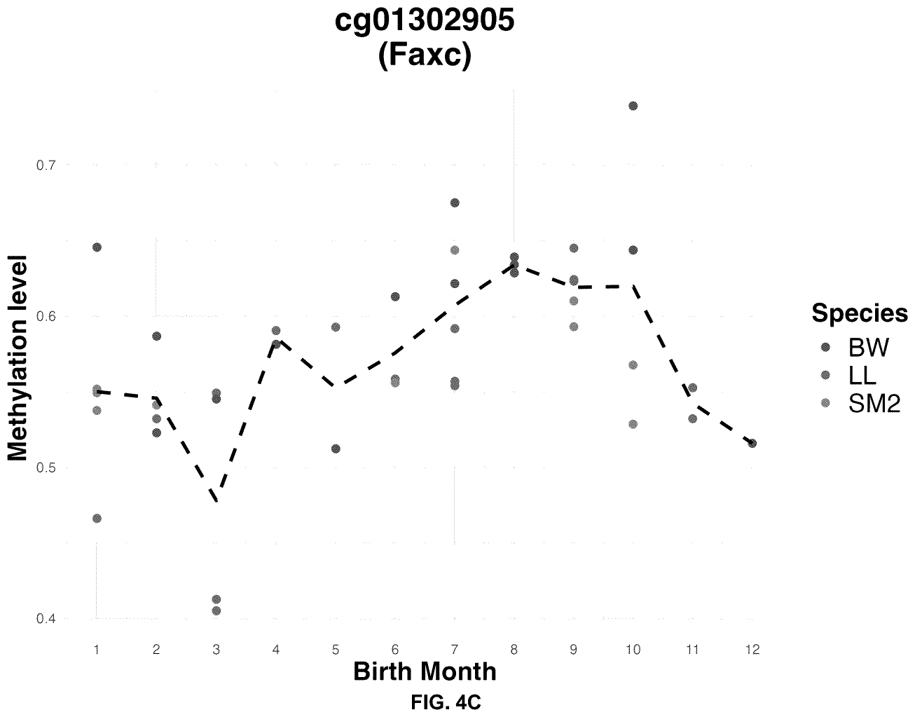

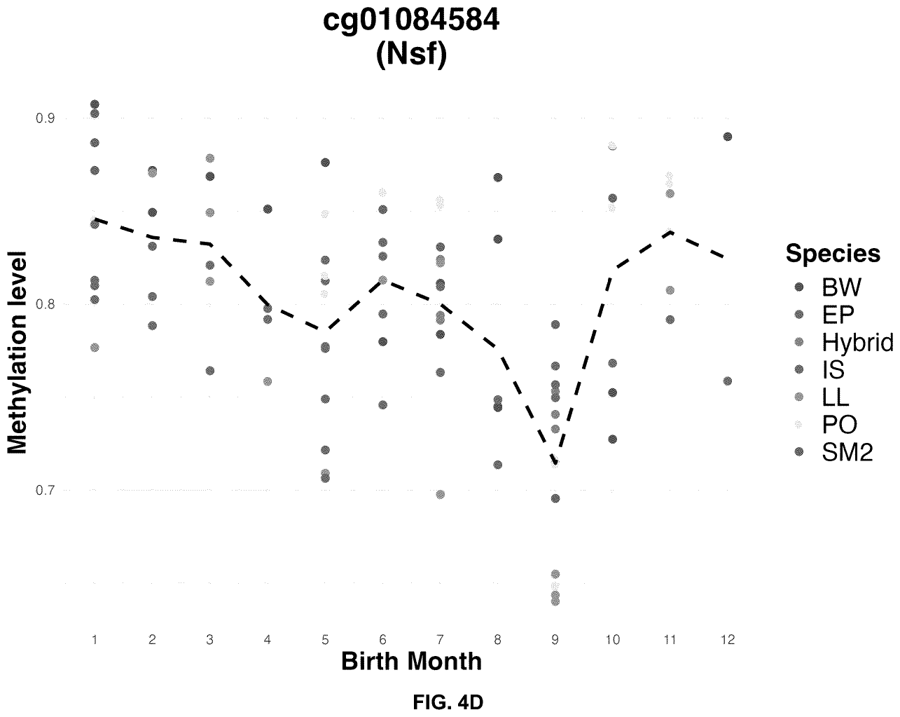

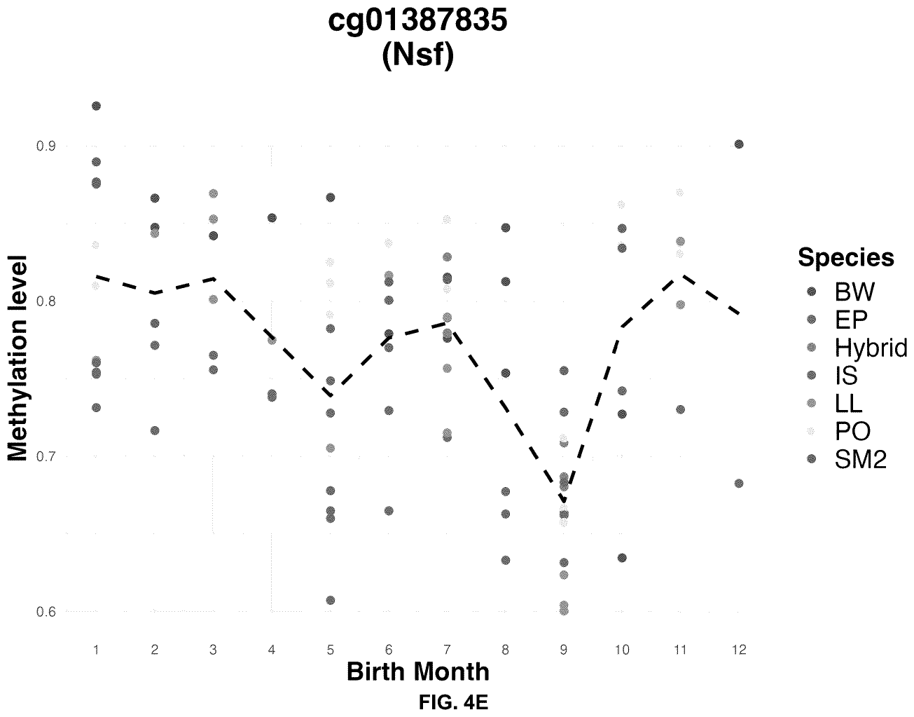

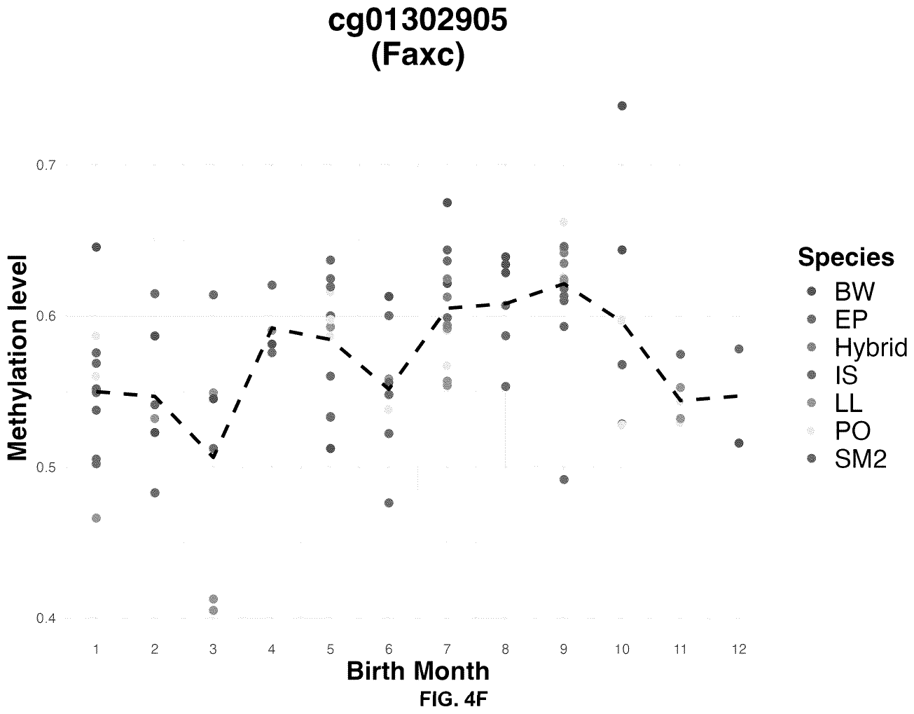

Subsequently, we tested how fitness in the 12-knots GAM varies between the different stocks. In the females, average fitness (R) throughout the year was significantly higher in the polygamous P. maniculatus bardii (BWck), compared to each of the monogamous P. californicus (IS) and P. eremicus (EP), and in the polygamous P. leucopus (LL) compared to the monogamous P. californicus (IS) (Huynh-Dam et al. [41] at FIG. 3a). Significant difference was also detected between the monogamous P. polionotus (PO) and P. californicus (IS) stocks (Huynh-Dam et al. [41] at FIG. 4a). In the males, significant differences were only detected between P. maniculatus bairdii (BW) and each of the other stocks (Huynh-Dam et al. [41] at FIG. 3a). This was surprising because BW and SM2 stocks correspond to different populations of the same species; nonetheless, this unexpected behavior of SM2 may be related to its high-altitude origin and/or the smaller number of breeding data available to extract robust conclusions. Nonetheless, a trend for better fitness in the polygamous BW and LL stocks compared to the monogamous species becomes apparent (Huynh-Dam et al. [41] at FIG. 3b). It is also noted that besides the breeding strategy, the difference between the P. leucopus and P. maniculatus, and P. eremicus and P. californicus, reflects their evolutionary history because the two first and the latter two species correspond to different clades of Peromyscus, with P. polionotus being more closely related to P. maniculatus than the other species (Turner et al., 2010). Therefore, the recorded differences may not be associated with monogamy but rather with the phylogenetic relevance of the stocks. When fitness in the 12-knots GAM model was compared between the males and the females within the same stock, no significant difference was revealed (Huynh-Dam et al. [41] at Additional file 1: FIG. S3).

### Preference for Summer Breeding by Winter-Born Parents in Polygamous Peromyscus

The prominent role of autumn and spring equinoxes in breeding patterns and methylation of Peromyscus suggest that each year can be divided into two seasons of winter and summer that are framed by the equinoxes and during which cumulative differences in breeding performance can be recorded. We tested this hypothesis by reanalyzing the available data and by considering the total number of offspring produced at each season by parents that were born either in the 6-month period from October to March (winter) or from April to September (summer). As shown in Huynh-Dam et al. [41] at FIG. 4a, in BW, LL, and SM2, summer litters were favored only when both parents were born in winter, while when parents were born in the summer, the offspring produced in winter and summer did not differ significantly. The effect was significant for BW (P=0.0005, Wilcoxon test) and LL (P=0.016, Wilcoxon test) and borderline insignificant for SM2 (P=0.06, Wilcoxon test) all of which are considered polygamous (Huynh-Dam et al. [41] at FIG. 4). When one parent was born in winter and the other in summer, this preference was abolished suggesting that both parents contributed to the effect noted (Huynh-Dam et al. [41] at Additional file 1: FIG. S4).

When the same analysis was performed for the species that show a tendency towards monogamous behavior (IS, PO and EP stocks), the pattern was not maintained. The number of offspring produced in winter or summer, by winter-born parents, did not differ considerably (Huynh-Dam et al. [41] at FIG. 4b and Additional file 1: FIG. S5). For the monogamous PO stock, an inversion in this trend was recorded according to which the summer-born parents favored the production of more litter in the summer but remained insignificant (P=0.06, Wilcoxon test). Thus, the preference for litters in summer, only by winter-born parents, was applicable to the polygamous stocks available to our facility. It is noted that monogamy evolved at least twice independently in deer mice, and therefore, alternative mechanisms may have been adopted [10, 12]. Furthermore, as already noted, the recorded differences may be indicative of phylogeny rather than breeding strategy.

Different latency for the production of the first litter did not account for the recorded differences in breeding performance because winter-born LL and SM2 males tended to produce offspring slightly, albeit significantly, earlier by about 15 days than their summer-born counterparts (Huynh-Dam et al. [41] at Additional file 1: FIG. S6). Furthermore, this slight difference would favor more offspring for winter-born animals.

Then we compared litter numbers in relation to birth season. Winter-born parents tended to produce more litters in summer than in winter, which effectively explains the higher number of offspring in summer than in winter recorded by winter-born parents. The effect was significant for LL (P=0.0156, Wilcoxon test) and BW (P=0.0015, Wilcoxon test) and borderline in SM2 (P=0.06, Wilcoxon test) (Huynh-Dam et al. [41] at Additional file 1: FIG. S7). The effect of litter number, being more in the summer for winter-born animals, was further enhanced in some stocks by an effect in litter sizes: in EP and PO, summer-born parents had smaller litters (P<0.05, Mann-Whitney test) in winter than in summer, while in BW and IS, winter-born animals had smaller litters in winter (P<0.05, Mann-Whitney test) (Huynh-Dam et al. [41] at Additional file 1: FIG. S7).

### Seasonal Effects in Estrus Cycle

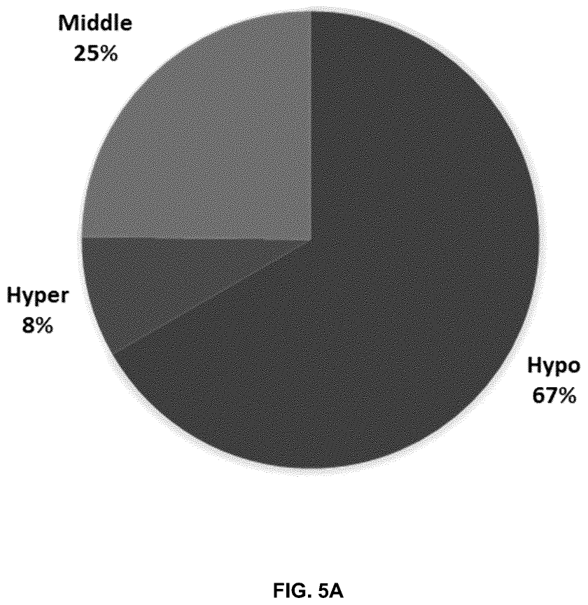

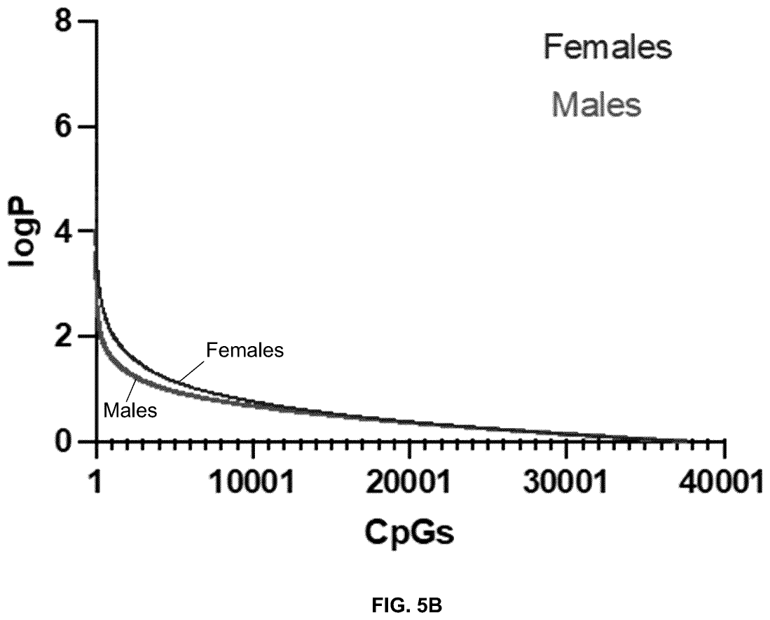

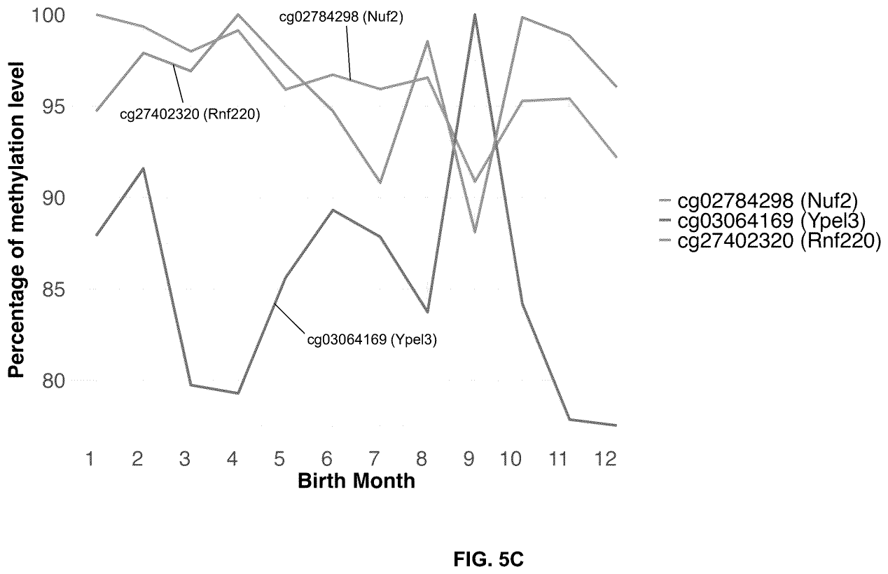

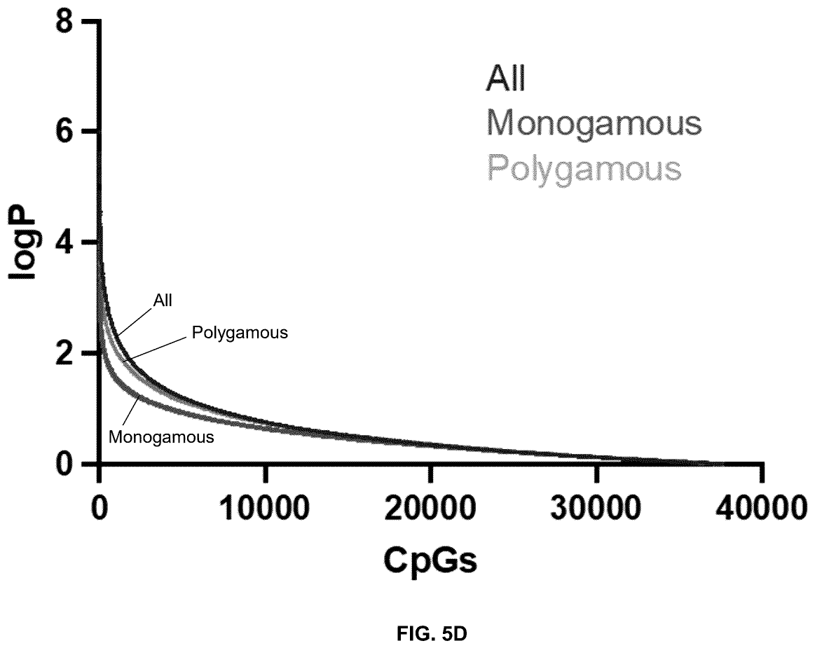

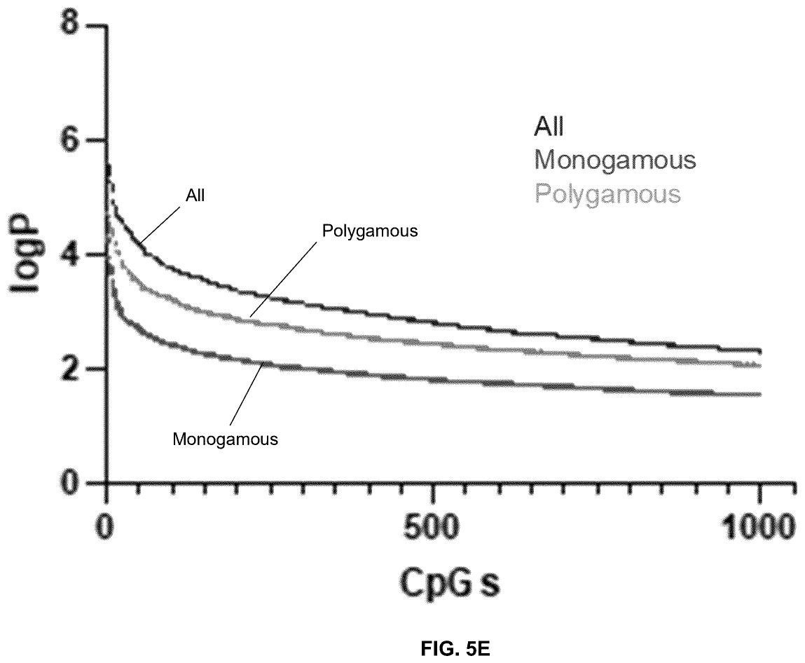

Different duration of the estrus cycle at different seasons may partially account for differences in breeding performance [13, 14]. Thus, we compared the duration of estrus cycle in winter, in winter-born, and summer-born females of polygamous P. maniculatus (BW) and monogamous P. californicus (IS). These species were selected because they exhibited marked differences in their breeding patterns. As shown in Huynh-Dam et al. [41] at FIG. 5, estrus cycle was significantly prolonged in polygamous P. maniculatus, but only in the winter-born females (Huynh-Dam et al. [41] at Additional file 1: FIG. S8). In the summer, no significant difference was detected in the estrus cycle length between the monogamous and polygamous summer-born or winter-born animals (Huynh-Dam et al. [41] at FIG. 5). Since animals born at different seasons will differ in age, we also compared the duration of the estrus cycle in BW differing one year in age, but no significant difference was found (Huynh-Dam et al. [41] at FIG. 5). Therefore, the prolongation of estrus cycle effectively explains, at least in part, the lower number of litters of winter-born polygamous Peromyscus in winter, since fewer pregnancies can be accomplished as compared to the summer.

### Circannual Methylation at Birth

Our observations suggest that deer mice can be impacted by the season of their birth in the animal facilities and adjust their reproductive behavior by a manner that exhibits specificity for their birth month. Thus, we hypothesized that the memory of birth season is associated with the imposition of an epigenetic imprint that persists throughout their reproductive lifespan. Furthermore, since breeding follows a circannual rhythm, we hypothesized that the epigenetic imprint should also follow circannual rhythm as well. To test these hypotheses, we explored if the presumed epigenetic memory involves season-dependent methylation by analyzing the methylome of Peromyscus, using circular statistics with linear modeling to explore epigenetic modifications across different times of the year. Specifically, the birth month is transformed into radians (sine and cosine transformations) to facilitate circular statistical analysis, capturing the cyclical nature of yearly events. The analysis involved ˜37,000 CpGs that were dispersed throughout the genome of all 6 Peromyscus stocks that were used for the analysis of breeding patterns. The methylation data used for this analysis were described previously, were retrieved, and reanalyzed in relation to the animals' birth season [15-17]. Only tail samples were used for this analysis (n=96). The distribution of mice in relation to their birth month is shown in FIG. 1.

To investigate seasonal patterns in DNA methylation associated with birth month, we analyzed the SeSAMe-normalized dataset, applying a statistical framework that accounts for periodic behavior. Birth month was transformed into sine and cosine functions to represent cyclical variation over time, a method commonly used in epidemiological studies to model seasonal effects [18]. Using a likelihood ratio test (LRT), we compared a full model incorporating these transformed terms with a reduced model that excluded them, adjusting for potential confounders: age, sex, and species [19]. From the analysis, we identified 377 CpGs that were methylated differentially throughout the year by a periodic manner (Table 2). The cutoff values used for this analysis were P<0.05 and FDRK<0.1. (Table 2) (Huynh-Dam et al. [141] at Additional file 2: Table S1). Prediction of biological function for the genes that exhibited circannual methylation at birth pointed to effects in transcriptional regulation, differentiation, and morphogenesis (FIGS. 2A and 2B). The highest correlation with seasonal methylation was shown for the gene NSF that encodes for the hexameric ATPase N-ethylmaleimide-sensitive factor (Huynh-Dam et al. [41] at Additional file 2: Table Si). NSF is involved in neurotransmitter release and was methylated differentially, by a season-depended manner in two intronic CpGs (cg01084584 and cg01387835, −log P=6 and 5.6, FDR=0.026 and 0.026, respectively) [20]. Noteworthy, NSF was recently identified in a sensory deficit screen in zebrafish [21].

TABLE 2
377 differentially methylated CpGs, statistical values, SEQ ID NOs of surrounding genomic
regions, genomic positions of the CpG start, and genomic positions of the CpG end.*

| | | | | SEQ ID NO | | Genomic | Genomic |
|---|---|---|---|---|---|---|---|
| | | | | of Genomic | Gene | Position of | Position of |
| CpG ID | logp | p | fdr | Region Seq. | Symbol | CpG Start | CpG End |
| cg01084584 | 6.001171387 | 9.97306E−07 | 0.025663636 | 1 | Nsf | 89311201 | 89311202 |
| cg16201028 | 5.625522915 | 2.36852E−06 | 0.025663636 | 2 | | | |
| cg01387835 | 5.548890726 | 2.82559E−06 | 0.025663636 | 3 | Nsf | 89311171 | 89311172 |
| cg13832100 | 5.484324122 | 3.27851E−06 | 0.025663636 | 4 | Nhej1 | 36949344 | 36949345 |
| cg00549985 | 5.414920203 | 3.84662E−06 | 0.025663636 | 5 | Cntfr | 62706463 | 62706464 |
| cg05992953 | 5.387186763 | 4.10028E−06 | 0.025663636 | 6 | Pbx3 | 5309897 | 5309898 |
| cg21544988 | 5.291721073 | 5.10833E−06 | 0.02740546 | 7 | Mecom | 14620581 | 14620582 |
| cg03064169 | 5.195100033 | 6.38116E−06 | 0.029954783 | 8 | Ypel3 | 117750720 | 117750721 |
| cg06916066 | 5.140080117 | 7.24302E−06 | 0.030222722 | 9 | Cachd1 | 118175835 | 118175836 |
| cg01302905 | 5.08552555 | 8.21248E−06 | 0.030841156 | 10 | Faxc | 43988404 | 43988405 |
| cg10295572 | 4.91150988 | 1.226E−05 | 0.040202489 | 11 | Hoxa5 | 70637699 | 70637700 |
| cg04337549 | 4.879938656 | 1.31844E−05 | 0.040202489 | 12 | Cbfa2t2 | 128959282 | 128959283 |
| cg14346040 | 4.833473261 | 1.46733E−05 | 0.040202489 | 13 | Nr1d1 | 84093607 | 84093608 |
| cg19553300 | 4.803324933 | 1.57281E−05 | 0.040202489 | 14 | Zeb2 | 23096848 | 23096849 |
| cg11666770 | 4.748448715 | 1.78464E−05 | 0.040202489 | 15 | Erfl | 24701217 | 24701218 |
| cg15234261 | 4.703498624 | 1.97925E−05 | 0.040202489 | 16 | Spry4 | 28632704 | 28632705 |
| cg11027534 | 4.684310677 | 2.06866E−05 | 0.040202489 | 17 | Dennd1a | 1496311 | 1496312 |
| cg22234802 | 4.679290322 | 2.09271E−05 | 0.040202489 | 18 | Rbm27 | 32092040 | 32092041 |
| cg16190348 | 4.657865041 | 2.19854E−05 | 0.040202489 | 19 | Bnc2 | 102128242 | 102128243 |
| cg00057767 | 4.656198731 | 2.20699E−05 | 0.040202489 | 20 | Zfp521 | 4701985 | 4701986 |
| cg24336374 | 4.635124882 | 2.31673E−05 | 0.040202489 | 21 | Runx1 | 76955596 | 76955597 |
| cg10389748 | 4.623031101 | 2.38215E−05 | 0.040202489 | 22 | Actrt2 | 158838430 | 158838431 |
| cg08714699 | 4.604925576 | 2.48356E−05 | 0.040202489 | 23 | Ldb3 | 3058791 | 3058792 |
| cg13449577 | 4.574164663 | 2.66585E−05 | 0.040202489 | 24 | Nek7 | 37562081 | 37562082 |
| cg10620157 | 4.57038572 | 2.68915E−05 | 0.040202489 | 25 | Nfix | 20336347 | 20336348 |
| cg18840480 | 4.541698332 | 2.87278E−05 | 0.040202489 | 26 | Kras | 156369753 | 156369754 |
| cg17983153 | 4.502243635 | 3.14598E−05 | 0.040202489 | 27 | Ubtf | 87796815 | 87796816 |
| cg15486794 | 4.499896619 | 3.16303E−05 | 0.040202489 | 28 | Ltbp3 | 139017344 | 139017345 |
| cg11702192 | 4.495618155 | 3.19435E−05 | 0.040202489 | 29 | Eya2 | 140847682 | 140847683 |
| cg26227235 | 4.493282004 | 3.21157E−05 | 0.040202489 | 30 | Tardbp | 165564752 | 165564753 |
| cg27443864 | 4.457380795 | 3.48834E−05 | 0.042258465 | 31 | Sp8 | 87344407 | 87344408 |
| cg16947402 | 4.439904204 | 3.63158E−05 | 0.042305913 | 32 | Msantd1 | 29343142 | 29343143 |
| cg06132693 | 4.403616654 | 3.94806E−05 | 0.042305913 | 33 | Cnnm2 | 177923066 | 177923067 |
| cg11023965 | 4.401582626 | 3.96659E−05 | 0.042305913 | 34 | Insrr | 65654810 | 65654811 |
| cg27504803 | 4.394514518 | 4.03167E−05 | 0.042305913 | 35 | Hdac4 | 13720594 | 13720595 |
| cg21767478 | 4.391952627 | 4.05553E−05 | 0.042305913 | 36 | Pak4 | 30694925 | 30694926 |
| cg23837023 | 4.376514763 | 4.20228E−05 | 0.042652031 | 37 | gene: | 6133478 | 6133479 |
| | | | | | ENSPEMG00000030557 | | |
| cg24251659 | 4.334132796 | 4.63305E−05 | 0.045666991 | 38 | Cux1 | 27650757 | 27650758 |
| cg12504775 | 4.323989196 | 4.74254E−05 | 0.045666991 | 39 | Tspan9 | 141295542 | 141295543 |
| cg11293950 | 4.28805783 | 5.1516E−05 | 0.048365801 | 40 | Pou3f1 | 138844970 | 138844971 |
| cg02784298 | 4.272379554 | 5.34097E−05 | 0.0485942 | 41 | Nuf2 | 66969626 | 66969627 |
| cg11269737 | 4.259487831 | 5.50189E−05 | 0.0485942 | 42 | | | |
| cg21834803 | 4.252708886 | 5.58845E−05 | 0.0485942 | 43 | Slit3 | 19393676 | 19393677 |
| cg00483633 | 4.232430428 | 5.85558E−05 | 0.0485942 | 44 | Socs7 | 82693928 | 82693929 |
| cg03436378 | 4.210276505 | 6.16203E−05 | 0.0485942 | 45 | Dlx1 | 48672071 | 48672072 |
| cg13192954 | 4.204507445 | 6.24443E−05 | 0.0485942 | 46 | Pdik1l | 148963162 | 148963163 |
| cg07537558 | 4.202007849 | 6.28047E−05 | 0.0485942 | 47 | Hoxa5 | 70645672 | 70645673 |
| cg21096298 | 4.199957415 | 6.31019E−05 | 0.0485942 | 48 | Hoxa5 | 70647141 | 70647142 |
| cg01856529 | 4.193858618 | 6.39943E−05 | 0.0485942 | 49 | Cbx5 | 69719259 | 69719260 |
| cg17468331 | 4.189101763 | 6.46991E−05 | 0.0485942 | 50 | Etv4 | 87264445 | 87264446 |
| cg09387286 | 4.161933044 | 6.88758E−05 | 0.050716933 | 51 | Evx1 | 70902741 | 70902742 |
| cg14945150 | 4.15342875 | 7.02379E−05 | 0.05072524 | 52 | Bcl9l | 39145849 | 39145850 |
| cg13817861 | 4.13317084 | 7.35918E−05 | 0.05128379 | 53 | Sorbs1 | 171081963 | 171081964 |
| cg10927578 | 4.132282329 | 7.37425E−05 | 0.05128379 | 54 | Magi1 | 108134422 | 108134423 |
| cg16946265 | 4.113290617 | 7.70388E−05 | 0.052602077 | 55 | Gata6 | 1690512 | 1690513 |
| cg06643163 | 4.077383829 | 8.36789E−05 | 0.054519122 | 56 | Tnr | 57909552 | 57909553 |
| cg00920973 | 4.074975826 | 8.41442E−05 | 0.054519122 | 57 | Ap3b1 | 57725526 | 57725527 |
| cg27596044 | 4.074679352 | 8.42017E−05 | 0.054519122 | 58 | Rara | 84288560 | 84288561 |
| cg07046041 | 4.058877851 | 8.73217E−05 | 0.055580998 | 59 | Gse1 | 49266735 | 49266736 |
| cg14213846 | 4.025041567 | 9.43971E−05 | 0.057634348 | 60 | Zc3h3 | 41526993 | 41526994 |
| cg08256518 | 3.997837946 | 0.000100499 | 0.057634348 | 61 | Foxp1 | 113889185 | 113889186 |
| cg23003419 | 3.997507209 | 0.000100576 | 0.057634348 | 62 | Nfia | 115274634 | 115274635 |
| cg23082716 | 3.992566836 | 0.000101726 | 0.057634348 | 63 | Cercam | 9831771 | 9831772 |
| cg09833169 | 3.991738427 | 0.000101921 | 0.057634348 | 64 | Bcl11a | 6970769 | 6970770 |
| cg22701693 | 3.990180352 | 0.000102287 | 0.057634348 | 65 | Gas1 | 135731054 | 135731055 |
| cg06236491 | 3.988719606 | 0.000102631 | 0.057634348 | 66 | Hoxa5 | 70643899 | 70643900 |
| cg27071972 | 3.987177034 | 0.000102997 | 0.057634348 | 67 | Gli3 | 92510393 | 92510394 |
| cg15343536 | 3.980181974 | 0.000104669 | 0.057634348 | 68 | Nfia | 115007662 | 115007663 |
| cg25793035 | 3.970414665 | 0.00010705 | 0.057634348 | 69 | Phc3 | 13483808 | 13483809 |
| cg00493843 | 3.968876776 | 0.000107429 | 0.057634348 | 70 | Mycl | 137284483 | 137284484 |
| cg12936001 | 3.936189127 | 0.000115827 | 0.059332168 | 71 | Csf1 | 83692566 | 83692567 |
| cg10709021 | 3.934665753 | 0.000116234 | 0.059332168 | 72 | Purg | 38036658 | 38036659 |
| cg06486970 | 3.930758582 | 0.000117285 | 0.059332168 | 73 | Tifab | 131458035 | 131458036 |
| cg21830760 | 3.922120714 | 0.000119641 | 0.059332168 | 74 | Gm20721 | 149909958 | 149909959 |
| cg03524646 | 3.921313413 | 0.000119863 | 0.059332168 | 75 | Zfp703 | 44229195 | 44229196 |
| cg15996281 | 3.915745059 | 0.00012141 | 0.059332168 | 76 | Zfp36l1 | 49739601 | 49739602 |
| cg00954841 | 3.914875259 | 0.000121654 | 0.059332168 | 77 | Tspan9 | 141295593 | 141295594 |
| cg03346007 | 3.878884198 | 0.000132165 | 0.063194208 | 78 | Ets1 | 28424032 | 28424033 |
| cg25326950 | 3.874619847 | 0.000133469 | 0.063194208 | 79 | Ptch1 | 137954841 | 137954842 |
| cg24822649 | 3.870888937 | 0.00013462 | 0.063194208 | 80 | Camkv | 103306141 | 103306142 |
| cg26957181 | 3.858029411 | 0.000138666 | 0.063588613 | 81 | Zcchc7 | 64988605 | 64988606 |
| cg00307913 | 3.85057476 | 0.000141067 | 0.063588613 | 82 | Cdk5rap3 | 82181563 | 82181564 |
| cg11040210 | 3.847744783 | 0.000141989 | 0.063588613 | 83 | Casz1 | 165455951 | 165455952 |
| cg18489996 | 3.846997565 | 0.000142234 | 0.063588613 | 84 | 1110051M20Rik | 67417158 | 67417159 |
| cg03509905 | 3.827574954 | 0.000148739 | 0.064260725 | 85 | Srrt | 28655176 | 28655177 |
| cg15446343 | 3.820970159 | 0.000151018 | 0.064260725 | 86 | Rnf144b | 122801914 | 122801915 |
| cg26154615 | 3.81462439 | 0.000153241 | 0.064260725 | 87 | Auts2 | 23071568 | 23071569 |
| cg00857016 | 3.812338614 | 0.00015405 | 0.064260725 | 88 | Pex14 | 165323229 | 165323230 |
| cg18818686 | 3.801622505 | 0.000157898 | 0.064260725 | 89 | Foxp4 | 57581230 | 57581231 |
| cg18289982 | 3.799550337 | 0.000158654 | 0.064260725 | 90 | Ap3b1 | 57670441 | 57670442 |
| cg10767126 | 3.799069129 | 0.000158829 | 0.064260725 | 91 | Asic2 | 67030364 | 67030365 |
| cg24370974 | 3.796071527 | 0.000159929 | 0.064260725 | 92 | Msh5 | 2624088 | 2624089 |
| cg24862683 | 3.791970012 | 0.000161447 | 0.064260725 | 93 | Tle4 | 146653635 | 146653636 |
| cg13992970 | 3.788424698 | 0.00016277 | 0.064260725 | 94 | Dennd1b | 38056708 | 38056709 |
| cg07608009 | 3.785969754 | 0.000163693 | 0.064260725 | 95 | Hoxa5 | 70647115 | 70647116 |
| cg21664672 | 3.78070062 | 0.000165691 | 0.064260725 | 96 | | | |
| cg07294852 | 3.779789453 | 0.000166039 | 0.064260725 | 97 | Mrm1 | 69754345 | 69754346 |
| cg06153489 | 3.772088822 | 0.00016901 | 0.064260725 | 98 | Gata3 | 79609491 | 79609492 |
| cg13489555 | 3.771075386 | 0.000169404 | 0.064260725 | 99 | Zc3h3 | 41527117 | 41527118 |
| cg09571697 | 3.752183351 | 0.000176936 | 0.06620439 | 100 | Lrba | 64346179 | 64346180 |
| cg08706469 | 3.744159725 | 0.000180235 | 0.06620439 | 101 | Hectd4 | 12636522 | 12636523 |
| cg10676571 | 3.742025725 | 0.000181123 | 0.06620439 | 102 | Auts2 | 22885074 | 22885075 |
| cg10202523 | 3.740932188 | 0.00018158 | 0.06620439 | 103 | Zc3h3 | 41526940 | 41526941 |
| cg15283949 | 3.734146426 | 0.000184439 | 0.066600338 | 104 | Dennd1a | 1804619 | 1804620 |
| cg14590156 | 3.72710598 | 0.000187454 | 0.066679417 | 105 | Hibadh | 71018123 | 71018124 |
| cg14589076 | 3.725358544 | 0.000188209 | 0.066679417 | 106 | Rnf220 | 132792805 | 132792806 |
| cg05062755 | 3.716833585 | 0.00019194 | 0.067365702 | 107 | Ddx31 | 10803673 | 10803674 |
| cg00711678 | 3.708753866 | 0.000195545 | 0.067594059 | 108 | Nedd4 | 54618015 | 54618016 |
| cg03969757 | 3.698578471 | 0.00020018 | 0.067594059 | 109 | Ep400 | 821052 | 821053 |
| cg02958263 | 3.696320577 | 0.000201224 | 0.067594059 | 110 | Rabgap1l | 58452381 | 58452382 |
| cg02897291 | 3.679156641 | 0.000209336 | 0.067594059 | 111 | Casz1 | 165455507 | 165455508 |
| cg14029942 | 3.677966085 | 0.00020991 | 0.067594059 | 112 | Pbx1 | 66253167 | 66253168 |
| cg07981074 | 3.677392792 | 0.000210188 | 0.067594059 | 113 | Rbm39 | 130874617 | 130874618 |
| cg25102951 | 3.674386496 | 0.000211648 | 0.067594059 | 114 | Pbx1 | 66269286 | 66269287 |
| cg09249640 | 3.673698331 | 0.000211983 | 0.067594059 | 115 | Foxp2 | 16169038 | 16169039 |
| cg12041264 | 3.667399287 | 0.00021508 | 0.067594059 | 116 | Hoxa5 | 70647102 | 70647103 |
| cg01548368 | 3.665931028 | 0.000215809 | 0.067594059 | 117 | Acaca | 69411869 | 69411870 |
| cg14525247 | 3.653073899 | 0.000222293 | 0.067594059 | 118 | Urm1 | 9842918 | 9842919 |
| cg13008977 | 3.650950363 | 0.000223383 | 0.067594059 | 119 | Slit3 | 19393667 | 19393668 |
| cg12459916 | 3.646090085 | 0.000225897 | 0.067594059 | 120 | Hoxa5 | 70647060 | 70647061 |
| cg25863798 | 3.645629111 | 0.000226137 | 0.067594059 | 121 | Mc2r | 58239095 | 58239096 |
| cg02810085 | 3.64525497 | 0.000226332 | 0.067594059 | 122 | Fubp3 | 8076188 | 8076189 |
| cg19638409 | 3.645071827 | 0.000226427 | 0.067594059 | 123 | Hoxa5 | 70647194 | 70647195 |
| cg19177967 | 3.644673776 | 0.000226635 | 0.067594059 | 124 | Insc | 106156457 | 106156458 |
| cg14032896 | 3.636301924 | 0.000231046 | 0.067594059 | 125 | Faf1 | 127411184 | 127411185 |
| cg25615319 | 3.634793213 | 0.00023185 | 0.067594059 | 126 | Trps1 | 12672511 | 12672512 |
| cg24028501 | 3.632155618 | 0.000233262 | 0.067594059 | 127 | Nek7 | 37562024 | 37562025 |
| cg17365200 | 3.631945796 | 0.000233375 | 0.067594059 | 128 | Wnk4 | 86644555 | 86644556 |
| cg13846137 | 3.627749806 | 0.000235641 | 0.067594059 | 129 | Emsy | 85657838 | 85657839 |
| cg03085335 | 3.623582328 | 0.000237913 | 0.067594059 | 130 | Kcnq1 | 134286611 | 134286612 |
| cg11988243 | 3.614166133 | 0.000243127 | 0.067594059 | 131 | | | |
| cg14639087 | 3.614009243 | 0.000243215 | 0.067594059 | 132 | Ccnl1 | 44853301 | 44853302 |
| cg12483863 | 3.613508547 | 0.000243496 | 0.067594059 | 133 | Faf1 | 127304306 | 127304307 |
| cg10991450 | 3.613144863 | 0.0002437 | 0.067594059 | 134 | Pkhd1 | 56328355 | 56328356 |
| cg11000391 | 3.609350525 | 0.000245838 | 0.067594059 | 135 | Pou4f3 | 32187696 | 32187697 |
| cg26080060 | 3.607627088 | 0.000246816 | 0.067594059 | 136 | Nrip1 | 60966646 | 60966647 |
| cg16197368 | 3.606182954 | 0.000247638 | 0.067594059 | 137 | Zfp281 | 35930851 | 35930852 |
| cg02368037 | 3.600812518 | 0.000250719 | 0.067594059 | 138 | Cdkal1 | 105037680 | 105037681 |
| cg13597158 | 3.594595348 | 0.000254334 | 0.067594059 | 139 | Casz1 | 165409602 | 165409603 |
| cg09753530 | 3.587902032 | 0.000258284 | 0.067594059 | 140 | Sp4 | 87121377 | 87121378 |
| cg13995516 | 3.585473418 | 0.000259733 | 0.067594059 | 141 | | | |
| cg25350245 | 3.585215237 | 0.000259887 | 0.067594059 | 142 | Msi1 | 5788365 | 5788366 |
| cg12358946 | 3.582558562 | 0.000261482 | 0.067594059 | 143 | gene: | 17399775 | 17399776 |
| | | | | | ENSPEMG00000017655 | | |
| cg26107500 | 3.582261164 | 0.000261661 | 0.067594059 | 144 | | | |
| cg11286786 | 3.581801924 | 0.000261938 | 0.067594059 | 145 | Casz1 | 165396084 | 165396085 |
| cg03523182 | 3.580394823 | 0.000262788 | 0.067594059 | 146 | Jade1 | 2387079 | 2387080 |
| cg11199141 | 3.572374001 | 0.000267686 | 0.068317093 | 147 | Hoxa5 | 70647063 | 70647064 |
| cg25140691 | 3.569865107 | 0.000269237 | 0.068317093 | 148 | | | |
| cg10852426 | 3.562865477 | 0.000273612 | 0.068961144 | 149 | gene: | 114745353 | 114745354 |
| | | | | | ENSPEMG00000025076 | | |
| cg01550545 | 3.559089671 | 0.000276001 | 0.069099558 | 150 | Nfia | 115274469 | 115274470 |
| cg24723713 | 3.547550557 | 0.000283432 | 0.06986826 | 151 | Psat1 | 147486817 | 147486818 |
| cg16029138 | 3.545838511 | 0.000284552 | 0.06986826 | 152 | Mtarc1 | 82303028 | 82303029 |
| cg27320080 | 3.545684842 | 0.000284653 | 0.06986826 | 153 | gene: | 47074565 | 47074566 |
| | | | | | ENSPEMG00000027258 | | |
| cg04601228 | 3.523889784 | 0.000299302 | 0.072987031 | 154 | Auts2 | 22885046 | 22885047 |
| cg18160481 | 3.51949181 | 0.000302349 | 0.073117224 | 155 | Son | 75942690 | 75942691 |
| cg11786975 | 3.516097366 | 0.000304721 | 0.073117224 | 156 | Casz1 | 165396121 | 165396122 |
| cg14481339 | 3.508178401 | 0.000310328 | 0.073117224 | 157 | Kcnj1 | 28078130 | 28078131 |
| cg23014792 | 3.50782059 | 0.000310584 | 0.073117224 | 158 | Pbx1 | 66077964 | 66077965 |
| cg27287578 | 3.507626285 | 0.000310723 | 0.073117224 | 159 | Rere | 163646731 | 163646732 |
| cg05522906 | 3.506516525 | 0.000311518 | 0.073117224 | 160 | Tfap2a | 115895994 | 115895995 |
| cg20093839 | 3.489978098 | 0.00032361 | 0.074383382 | 161 | Meis1 | 12270257 | 12270258 |
| cg15647529 | 3.489079452 | 0.00032428 | 0.074383382 | 162 | Mlf1 | 46039583 | 46039584 |
| cg02042105 | 3.485542679 | 0.000326932 | 0.074383382 | 163 | | | |
| cg00850663 | 3.482885226 | 0.000328939 | 0.074383382 | 164 | Hoxa5 | 70647118 | 70647119 |
| cg11327853 | 3.479660723 | 0.00033139 | 0.074383382 | 165 | Arhgap26 | 29432486 | 29432487 |
| cg26993000 | 3.478999717 | 0.000331895 | 0.074383382 | 166 | Glis1 | 124919937 | 124919938 |
| cg01744051 | 3.477213917 | 0.000333262 | 0.074383382 | 167 | Nek7 | 37558871 | 37558872 |
| cg26112292 | 3.473770021 | 0.000335915 | 0.074383382 | 168 | gene: | 6133244 | 6133245 |
| | | | | | ENSPEMG00000030557 | | |
| cg00645382 | 3.472284041 | 0.000337067 | 0.074383382 | 169 | Auts2 | 22884956 | 22884957 |
| cg05247990 | 3.467714592 | 0.000340632 | 0.074383382 | 170 | Bnc2 | 102489783 | 102489784 |
| cg17890349 | 3.467173074 | 0.000341057 | 0.074383382 | 171 | Eya2 | 140847635 | 140847636 |
| cg03901389 | 3.466366603 | 0.000341691 | 0.074383382 | 172 | Gpr180 | 109895405 | 109895406 |
| cg19379020 | 3.461443564 | 0.000345586 | 0.074383382 | 173 | Inpp4a | 38044309 | 38044310 |
| cg26677826 | 3.458837699 | 0.000347666 | 0.074383382 | 174 | Rnf220 | 132696445 | 132696446 |
| cg12674712 | 3.455922719 | 0.000350007 | 0.074383382 | 175 | Bcl11b | 77056056 | 77056057 |
| cg17244219 | 3.453171357 | 0.000352232 | 0.074383382 | 176 | Hoxa5 | 70647093 | 70647094 |
| cg10420536 | 3.452876659 | 0.000352471 | 0.074383382 | 177 | Ranbp17 | 14484551 | 14484552 |
| cg16926809 | 3.452760278 | 0.000352565 | 0.074383382 | 178 | Casz1 | 165396093 | 165396094 |
| cg08912986 | 3.445836368 | 0.000358231 | 0.075156544 | 179 | Hoxa5 | 70643927 | 70643928 |
| cg14309106 | 3.439879789 | 0.000363179 | 0.075358113 | 180 | Map4 | 104803784 | 104803785 |
| cg26307043 | 3.439847613 | 0.000363205 | 0.075358113 | 181 | Dchs1 | 96857135 | 96857136 |
| cg25404370 | 3.429917493 | 0.000371606 | 0.07667739 | 182 | Wnk4 | 86644600 | 86644601 |
| cg26276608 | 3.427015814 | 0.000374097 | 0.076769604 | 183 | Pds5a | 58872397 | 58872398 |
| cg03472582 | 3.422825911 | 0.000377724 | 0.076932673 | 184 | Satb1 | 66193737 | 66193738 |
| cg12387075 | 3.419857124 | 0.000380314 | 0.076932673 | 185 | Arhgap15 | 22357538 | 22357539 |
| cg25937299 | 3.417882637 | 0.000382048 | 0.076932673 | 186 | Palld | 11453673 | 11453674 |
| cg04499392 | 3.416703772 | 0.000383086 | 0.076932673 | 187 | Dph6 | 88434458 | 88434459 |
| cg27402320 | 3.413687678 | 0.000385756 | 0.077056748 | 188 | Rnf220 | 132823935 | 132823936 |
| cg07843244 | 3.407697424 | 0.000391113 | 0.07771359 | 189 | Sall3 | 70818270 | 70818271 |
| cg23936031 | 3.398185536 | 0.000399774 | 0.078692551 | 190 | Hoxa5 | 70647135 | 70647136 |
| cg08145628 | 3.397689211 | 0.000400231 | 0.078692551 | 191 | Stk38 | 6205266 | 6205267 |
| cg06670018 | 3.393351668 | 0.000404248 | 0.079068465 | 192 | Dlx1 | 48672189 | 48672190 |
| cg04088732 | 3.390494536 | 0.000406917 | 0.079177969 | 193 | Lrmda | 33732844 | 33732845 |
| cg13472501 | 3.384021922 | 0.000413027 | 0.079840909 | 194 | Ola1 | 50370013 | 50370014 |
| cg17831448 | 3.382396118 | 0.000414576 | 0.079840909 | 195 | Faah | 131237916 | 131237917 |
| cg05415875 | 3.379996339 | 0.000416873 | 0.079873698 | 196 | Pex14 | 165323175 | 165323176 |
| cg11804981 | 3.375425047 | 0.000421284 | 0.080309131 | 197 | 6430628N08Rik | 131167245 | 131167246 |
| cg22409694 | 3.371616248 | 0.000424995 | 0.080318002 | 198 | Lmo4 | 119328239 | 119328240 |
| cg00205619 | 3.370990232 | 0.000425608 | 0.080318002 | 199 | Cdkal1 | 105037624 | 105037625 |
| cg21805940 | 3.361297696 | 0.000435213 | 0.080560213 | 200 | Pcca | 105149494 | 105149495 |
| cg14425465 | 3.354804652 | 0.000441769 | 0.080560213 | 201 | Uri1 | 33678217 | 33678218 |
| cg10196526 | 3.350428742 | 0.000446243 | 0.080560213 | 202 | Cadps | 18659736 | 18659737 |
| cg15193680 | 3.349707901 | 0.000446984 | 0.080560213 | 203 | Tcf4 | 59119275 | 59119276 |
| cg24646022 | 3.349260601 | 0.000447445 | 0.080560213 | 204 | Ehbp1 | 9203196 | 9203197 |
| cg14847184 | 3.347045744 | 0.000449732 | 0.080560213 | 205 | Casz1 | 165455421 | 165455422 |
| cg24440735 | 3.345262821 | 0.000451583 | 0.080560213 | 206 | Maf | 44209719 | 44209720 |
| cg05067709 | 3.345172738 | 0.000451676 | 0.080560213 | 207 | Lrmda | 34159560 | 34159561 |
| cg16117415 | 3.342714531 | 0.00045424 | 0.080560213 | 208 | Zmiz1 | 31182276 | 31182277 |
| cg17604804 | 3.342682451 | 0.000454274 | 0.080560213 | 209 | Ptch1 | 137923102 | 137923103 |
| cg02354041 | 3.341580653 | 0.000455428 | 0.080560213 | 210 | Pbx3 | 5185068 | 5185069 |
| cg00384342 | 3.341294273 | 0.000455728 | 0.080560213 | 211 | Dync1i1 | 8071757 | 8071758 |
| cg10755942 | 3.33994332 | 0.000457148 | 0.080560213 | 212 | Meis1 | 12440900 | 12440901 |
| cg27608858 | 3.338817114 | 0.000458335 | 0.080560213 | 213 | Zfp507 | 35535563 | 35535564 |
| cg23164054 | 3.338121823 | 0.000459069 | 0.080560213 | 214 | Spred1 | 90668965 | 90668966 |
| cg03598181 | 3.332976807 | 0.00046454 | 0.081141108 | 215 | Bcl11a | 6970940 | 6970941 |
| cg00784860 | 3.329201002 | 0.000468596 | 0.081221395 | 216 | Emsy | 85657824 | 85657825 |
| cg18116969 | 3.324016031 | 0.000474224 | 0.081221395 | 217 | Grb14 | 41931827 | 41931828 |
| cg05185512 | 3.323987298 | 0.000474256 | 0.081221395 | 218 | Meis1 | 12074128 | 12074129 |
| cg12287916 | 3.323785702 | 0.000474476 | 0.081221395 | 219 | Tmem265 | 118517568 | 118517569 |
| cg15634098 | 3.321581373 | 0.00047689 | 0.081221395 | 220 | Rapgef2 | 56953808 | 56953809 |
| cg16699500 | 3.318316217 | 0.000480489 | 0.081221395 | 221 | Tbca | 58034720 | 58034721 |
| cg12653841 | 3.317938361 | 0.000480908 | 0.081221395 | 222 | Psd3 | 5878100 | 5878101 |
| cg18091117 | 3.316680894 | 0.000482302 | 0.081221395 | 223 | Hoxa5 | 70643793 | 70643794 |
| cg16821706 | 3.312743247 | 0.000486695 | 0.081595261 | 224 | Ola1 | 50370035 | 50370036 |
| cg10839301 | 3.309843248 | 0.000489956 | 0.081776861 | 225 | Dph6 | 88640003 | 88640004 |
| cg08113048 | 3.30027281 | 0.000500873 | 0.083229053 | 226 | Rnf220 | 132683250 | 132683251 |
| cg01761301 | 3.296167815 | 0.000505629 | 0.083649342 | 227 | Oga | 176801615 | 176801616 |
| cg10769266 | 3.294135214 | 0.000508001 | 0.083673154 | 228 | Rngtt | 54953409 | 54953410 |
| cg15561172 | 3.291933329 | 0.000510583 | 0.083731215 | 229 | Mlf1 | 45988818 | 45988819 |
| cg27528213 | 3.287961263 | 0.000515275 | 0.084133141 | 230 | Fam172a | 75834632 | 75834633 |
| cg23994088 | 3.282108726 | 0.000522265 | 0.084731864 | 231 | Hoxa5 | 70647692 | 70647693 |
| cg25569421 | 3.281056893 | 0.000523532 | 0.084731864 | 232 | Srsf11 | 133521263 | 133521264 |
| cg24850996 | 3.278655449 | 0.000526435 | 0.084731864 | 233 | Chic2 | 67997837 | 67997838 |
| cg21296320 | 3.27537994 | 0.00053042 | 0.084731864 | 234 | Zeb2 | 23148195 | 23148196 |
| cg27596519 | 3.27238367 | 0.000534092 | 0.084731864 | 235 | Ppox | 69222336 | 69222337 |
| cg22911887 | 3.270740771 | 0.000536117 | 0.084731864 | 236 | Gata2 | 100997462 | 100997463 |
| cg02197333 | 3.27068853 | 0.000536181 | 0.084731864 | 237 | Trp63 | 12358174 | 12358175 |
| cg18044369 | 3.267463155 | 0.000540178 | 0.084731864 | 238 | Zbtb16 | 43582223 | 43582224 |
| cg22539736 | 3.264676571 | 0.000543655 | 0.084731864 | 239 | Atxn2l | 117387522 | 117387523 |
| cg19327615 | 3.263839967 | 0.000544703 | 0.084731864 | 240 | Rin2 | 119995575 | 119995576 |
| cg08435157 | 3.263000125 | 0.000545758 | 0.084731864 | 241 | Nptn | 53621051 | 53621052 |
| cg01761369 | 3.262473832 | 0.000546419 | 0.084731864 | 242 | Dab2ip | 3804356 | 3804357 |
| cg01326050 | 3.259501053 | 0.000550173 | 0.084731864 | 243 | Pth1r | 105562072 | 105562073 |
| cg25041838 | 3.254732578 | 0.000556247 | 0.084731864 | 244 | Pcca | 105149466 | 105149467 |
| cg01243266 | 3.253479827 | 0.000557854 | 0.084731864 | 245 | Erfl | 24693371 | 24693372 |
| cg23547813 | 3.252986418 | 0.000558488 | 0.084731864 | 246 | Nfix | 20336331 | 20336332 |
| cg10088405 | 3.252105903 | 0.000559621 | 0.084731864 | 247 | Il12a | 47328737 | 47328738 |
| cg18669384 | 3.251368736 | 0.000560572 | 0.084731864 | 248 | Arid5b | 19547437 | 19547438 |
| cg26767614 | 3.246684021 | 0.000566651 | 0.084731864 | 249 | Nfix | 20336222 | 20336223 |
| cg22213242 | 3.246540546 | 0.000566839 | 0.084731864 | 250 | Cd248 | 138338482 | 138338483 |
| cg26293238 | 3.243723683 | 0.000570527 | 0.084731864 | 251 | gene: | 26287417 | 26287418 |
| | | | | | ENSPEMG00000025041 | | |
| cg19751323 | 3.243134125 | 0.000571302 | 0.084731864 | 252 | Cnnm2 | 178016888 | 178016889 |
| cg10404717 | 3.241974835 | 0.000572829 | 0.084731864 | 253 | Urm1 | 9842855 | 9842856 |
| cg11812837 | 3.241775723 | 0.000573092 | 0.084731864 | 254 | Zeb2 | 23134323 | 23134324 |
| cg17359136 | 3.235803863 | 0.000581027 | 0.085568153 | 255 | Bahcc1 | 107656281 | 107656282 |
| cg02991558 | 3.231633534 | 0.000586633 | 0.086056306 | 256 | | | |
| cg02815776 | 3.226157352 | 0.000594077 | 0.086809195 | 257 | Tspan9 | 141351101 | 141351102 |
| cg06795337 | 3.221468211 | 0.000600526 | 0.086976374 | 258 | Prl5a1 | 103985418 | 103985419 |
| cg08591301 | 3.221058065 | 0.000601093 | 0.086976374 | 259 | Gbe1 | 55187384 | 55187385 |
| cg01273518 | 3.220281558 | 0.000602169 | 0.086976374 | 260 | Mrps9 | 32406457 | 32406458 |
| cg18927470 | 3.214886236 | 0.000609697 | 0.087260819 | 261 | Tle4 | 146270546 | 146270547 |
| cg06779064 | 3.214200729 | 0.00061066 | 0.087260819 | 262 | Tnrc6a | 114114923 | 114114924 |
| cg02476543 | 3.212460571 | 0.000613111 | 0.087260819 | 263 | Copb2 | 93420066 | 93420067 |
| cg00860838 | 3.211974371 | 0.000613798 | 0.087260819 | 264 | Lhfpl4 | 126994262 | 126994263 |
| cg19996711 | 3.210591041 | 0.000615756 | 0.087260819 | 265 | Ambra1 | 67903267 | 67903268 |
| cg17950076 | 3.20711856 | 0.0006207 | 0.087630644 | 266 | Sp3 | 50197481 | 50197482 |
| cg03454056 | 3.202707397 | 0.000627036 | 0.087873737 | 267 | Bnc2 | 102253087 | 102253088 |
| cg17640388 | 3.202662311 | 0.000627101 | 0.087873737 | 268 | Cyp26b1 | 98707340 | 98707341 |
| cg10081375 | 3.19832958 | 0.000633389 | 0.088424852 | 269 | Mnt | 59634580 | 59634581 |
| cg13144031 | 3.193612236 | 0.000640306 | 0.088425214 | 270 | Poll | 176584750 | 176584751 |
| cg01757168 | 3.192984027 | 0.000641233 | 0.088425214 | 271 | Gata2 | 100997635 | 100997636 |
| cg10718793 | 3.192211192 | 0.000642375 | 0.088425214 | 272 | Tcf4 | 58983052 | 58983053 |
| cg17167646 | 3.191917433 | 0.00064281 | 0.088425214 | 273 | Zfp703 | 43971005 | 43971006 |
| cg18783345 | 3.187912336 | 0.000648765 | 0.088490655 | 274 | Auts2 | 22885069 | 22885070 |
| cg00236139 | 3.187852768 | 0.000648854 | 0.088490655 | 275 | Rnf220 | 132717885 | 132717886 |
| cg04571367 | 3.186849708 | 0.000650355 | 0.088490655 | 276 | Macf1 | 137739631 | 137739632 |
| cg22188736 | 3.183955619 | 0.000654703 | 0.088760719 | 277 | Tifab | 131458021 | 131458022 |
| cg15044533 | 3.18173317 | 0.000658062 | 0.088895185 | 278 | | | |
| cg00314427 | 3.178359737 | 0.000663194 | 0.089267272 | 279 | Fosb | 20277029 | 20277030 |
| cg26237529 | 3.1734392 | 0.00067075 | 0.089716545 | 280 | Lrmda | 34167625 | 34167626 |
| cg20075348 | 3.172919989 | 0.000671553 | 0.089716545 | 281 | gene: | 6133210 | 6133211 |
| | | | | | ENSPEMG00000030557 | | |
| cg07910324 | 3.171534552 | 0.000673698 | 0.089716545 | 282 | Jag1 | 111537777 | 111537778 |
| cg22362271 | 3.168500479 | 0.000678421 | 0.089920595 | 283 | Hibadh | 71018153 | 71018154 |
| cg23779931 | 3.166253779 | 0.00068194 | 0.089920595 | 284 | Grm6 | 34455118 | 34455119 |
| cg00054169 | 3.16309273 | 0.000686922 | 0.089920595 | 285 | Hoxa5 | 70647262 | 70647263 |
| cg18784090 | 3.162704604 | 0.000687536 | 0.089920595 | 286 | Kazald1 | 176093152 | 176093153 |
| cg10172407 | 3.158608976 | 0.00069405 | 0.089920595 | 287 | Btrc | 176335425 | 176335426 |
| cg16790383 | 3.156801618 | 0.000696945 | 0.089920595 | 288 | Sf1 | 139696809 | 139696810 |
| cg17985457 | 3.153112131 | 0.000702891 | 0.089920595 | 289 | gene: | 6718574 | 6718575 |
| | | | | | ENSPEMG00000030557 | | |
| cg03306513 | 3.15285173 | 0.000703312 | 0.089920595 | 290 | Slit3 | 19393215 | 19393216 |
| cg03637267 | 3.150621448 | 0.000706933 | 0.089920595 | 291 | R3hcc1l | 173485880 | 173485881 |
| cg06242209 | 3.147509481 | 0.000712017 | 0.089920595 | 292 | Slc35b3 | 114544045 | 114544046 |
| cg25249425 | 3.147320505 | 0.000712327 | 0.089920595 | 293 | Sp4 | 87121351 | 87121352 |
| cg02192891 | 3.146536836 | 0.000713614 | 0.089920595 | 294 | Hoxa5 | 70647056 | 70647057 |
| cg21938659 | 3.145965774 | 0.000714553 | 0.089920595 | 295 | Faxc | 43988466 | 43988467 |
| cg04220619 | 3.142967786 | 0.000719502 | 0.089920595 | 296 | Mark2 | 140494622 | 140494623 |
| cg16044855 | 3.141466009 | 0.000721995 | 0.089920595 | 297 | Npepps | 82527513 | 82527514 |
| cg23940242 | 3.139639115 | 0.000725038 | 0.089920595 | 298 | Fign | 41282656 | 41282657 |
| cg19559133 | 3.138143526 | 0.000727539 | 0.089920595 | 299 | Cercam | 9831908 | 9831909 |
| cg24895764 | 3.137745131 | 0.000728207 | 0.089920595 | 300 | Sugct | 93995955 | 93995956 |
| cg14019542 | 3.136088068 | 0.000730991 | 0.089920595 | 301 | Pkp4 | 36434160 | 36434161 |
| cg19088488 | 3.135794103 | 0.000731486 | 0.089920595 | 302 | Bcl11b | 77056053 | 77056054 |
| cg17552603 | 3.134363208 | 0.0007339 | 0.089920595 | 303 | Fto | 13114646 | 13114647 |
| cg01927175 | 3.132949473 | 0.000736293 | 0.089920595 | 304 | Ranbp17 | 14378415 | 14378416 |
| cg03638659 | 3.131353941 | 0.000739003 | 0.089920595 | 305 | Ebf2 | 58833414 | 58833415 |
| cg01182628 | 3.131212301 | 0.000739244 | 0.089920595 | 306 | Maml3 | 32092364 | 32092365 |
| cg06188746 | 3.130724629 | 0.000740074 | 0.089920595 | 307 | Hoxa5 | 70643761 | 70643762 |
| cg07386222 | 3.130635254 | 0.000740227 | 0.089920595 | 308 | Zswim8 | 35683712 | 35683713 |
| cg21385340 | 3.13001121 | 0.000741291 | 0.089920595 | 309 | Emc2 | 19319052 | 19319053 |
| cg09594074 | 3.129435336 | 0.000742275 | 0.089920595 | 310 | gene: | 6657604 | 6657605 |
| | | | | | ENSPEMG00000030557 | | |
| cg12791866 | 3.125587588 | 0.00074888 | 0.090429103 | 311 | Chd7 | 31019585 | 31019586 |
| cg19701703 | 3.124116326 | 0.000751422 | 0.090435538 | 312 | Atxn2l | 117387311 | 117387312 |
| cg00731024 | 3.120467271 | 0.000757762 | 0.090435538 | 313 | Dach1 | 88417668 | 88417669 |
| cg20215112 | 3.119287078 | 0.000759824 | 0.090435538 | 314 | C1ql1 | 88468197 | 88468198 |
| cg09916849 | 3.118508502 | 0.000761187 | 0.090435538 | 315 | Bnc2 | 102276336 | 102276337 |
| cg24713149 | 3.11823869 | 0.00076166 | 0.090435538 | 316 | | | |
| cg17797918 | 3.117257815 | 0.000763382 | 0.090435538 | 317 | Mctp1 | 76810499 | 76810500 |
| cg05338309 | 3.115616361 | 0.000766273 | 0.090492528 | 318 | Faah | 131237870 | 131237871 |
| cg26487099 | 3.113119215 | 0.000770692 | 0.090729038 | 319 | Tbca | 58035116 | 58035117 |
| cg13425116 | 3.105235167 | 0.000784811 | 0.091596528 | 320 | Zfp64 | 144134950 | 144134951 |
| cg19384462 | 3.104401604 | 0.000786318 | 0.091596528 | 321 | Nkx6-3 | 47867050 | 47867051 |
| cg12347497 | 3.103406592 | 0.000788122 | 0.091596528 | 322 | Wwox | 43729752 | 43729753 |
| cg02515217 | 3.103375895 | 0.000788178 | 0.091596528 | 323 | Tubd1 | 71749687 | 71749688 |
| cg11982879 | 3.10223218 | 0.000790256 | 0.091596528 | 324 | Dhx15 | 46301846 | 46301847 |
| cg03538828 | 3.096781011 | 0.000800238 | 0.09229816 | 325 | Efna5 | 3686198 | 3686199 |
| cg12649161 | 3.09624556 | 0.000801225 | 0.09229816 | 326 | Agap1 | 11176846 | 11176847 |
| cg14736210 | 3.092514809 | 0.000808137 | 0.092809758 | 327 | | | |
| cg25500822 | 3.089913363 | 0.000812993 | 0.092889998 | 328 | Irx3 | 12869685 | 12869686 |
| cg26917253 | 3.089491352 | 0.000813783 | 0.092889998 | 329 | Fam181a | 72146483 | 72146484 |
| cg16339305 | 3.085186989 | 0.000821889 | 0.093530934 | 330 | Prdm1 | 6595255 | 6595256 |
| cg09541874 | 3.083054889 | 0.000825934 | 0.093677651 | 331 | Sema3f | 103015283 | 103015284 |
| cg26562677 | 3.079655957 | 0.000832423 | 0.093677651 | 332 | Rps24 | 31482282 | 31482283 |
| cg09606715 | 3.079522477 | 0.000832679 | 0.093677651 | 333 | Jade1 | 2387073 | 2387074 |
| cg10677704 | 3.078305709 | 0.000835015 | 0.093677651 | 334 | Ewsr1 | 24968673 | 24968674 |
| cg05202136 | 3.07693525 | 0.000837654 | 0.093677651 | 335 | Msantd1 | 29343135 | 29343136 |
| cg21431064 | 3.074701591 | 0.000841973 | 0.093677651 | 336 | Gpr180 | 110044582 | 110044583 |
| cg12903634 | 3.074048971 | 0.00084324 | 0.093677651 | 337 | Zfp800 | 41454826 | 41454827 |
| cg05562961 | 3.070949235 | 0.00084928 | 0.093677651 | 338 | Rnf220 | 132807690 | 132807691 |
| cg17644722 | 3.070879954 | 0.000849415 | 0.093677651 | 339 | Npas3 | 17735836 | 17735837 |
| cg27500609 | 3.070394193 | 0.000850366 | 0.093677651 | 340 | Celf4 | 16027941 | 16027942 |
| cg22862920 | 3.070265832 | 0.000850617 | 0.093677651 | 341 | Ass1 | 8109333 | 8109334 |
| cg23861489 | 3.068687776 | 0.000853714 | 0.09374375 | 342 | Myog | 33212245 | 33212246 |
| cg02248486 | 3.066978648 | 0.00085708 | 0.093759343 | 343 | Hoxa5 | 70647198 | 70647199 |
| cg01643535 | 3.066083202 | 0.000858849 | 0.093759343 | 344 | Ncdn | 141190383 | 141190384 |
| cg06367756 | 3.061924847 | 0.000867112 | 0.094152844 | 345 | Hdac8 | 75784335 | 75784336 |
| cg20846390 | 3.060381876 | 0.000870198 | 0.094152844 | 346 | Tmem68 | 36531442 | 36531443 |
| cg16663789 | 3.060131447 | 0.0008707 | 0.094152844 | 347 | Nfia | 115274451 | 115274452 |
| cg26993398 | 3.058728823 | 0.000873517 | 0.094152844 | 348 | Auts2 | 22884885 | 22884886 |
| cg25573671 | 3.057997331 | 0.000874989 | 0.094152844 | 349 | | | |
| cg07982366 | 3.054845283 | 0.000881363 | 0.09456771 | 350 | Syt14 | 91986949 | 91986950 |
| cg05870606 | 3.050733258 | 0.000889747 | 0.09519537 | 351 | Casz1 | 165547293 | 165547294 |
| cg01634979 | 3.048706588 | 0.000893909 | 0.095368939 | 352 | Pdss2 | 7685447 | 7685448 |
| cg27244752 | 3.046759963 | 0.000897925 | 0.095517529 | 353 | Pbx3 | 5185027 | 5185028 |
| cg07382457 | 3.045257366 | 0.000901037 | 0.095517529 | 354 | Nek7 | 37562085 | 37562086 |
| cg11381764 | 3.044344768 | 0.000902932 | 0.095517529 | 355 | Gli3 | 92510263 | 92510264 |
| cg25149789 | 3.041177584 | 0.000909541 | 0.095870996 | 356 | S1pr3 | 126328582 | 126328583 |
| cg18476882 | 3.04015474 | 0.000911686 | 0.095870996 | 357 | | | |
| cg05167704 | 3.037283383 | 0.000917734 | 0.095870996 | 358 | Fign | 41520086 | 41520087 |
| cg12433048 | 3.036593098 | 0.000919193 | 0.095870996 | 359 | Csnk1a1 | 50938657 | 50938658 |
| cg26441980 | 3.035718884 | 0.000921046 | 0.095870996 | 360 | Skap2 | 70310196 | 70310197 |
| cg16743922 | 3.035461761 | 0.000921591 | 0.095870996 | 361 | Fbxw4 | 176660681 | 176660682 |
| cg10503670 | 3.030702891 | 0.000931745 | 0.096634692 | 362 | Zc2hc1b | 41645165 | 41645166 |
| cg24052926 | 3.02910831 | 0.000935172 | 0.096634692 | 363 | Fgf11 | 53922927 | 53922928 |
| cg01961215 | 3.02842175 | 0.000936652 | 0.096634692 | 364 | Nfia | 115007646 | 115007647 |
| cg06023837 | 3.024656807 | 0.000944807 | 0.097209012 | 365 | Smg6 | 59847087 | 59847088 |
| cg13084522 | 3.022551872 | 0.000949398 | 0.097336543 | 366 | Atxn1l | 38228008 | 38228009 |
| cg08886274 | 3.021714218 | 0.000951231 | 0.097336543 | 367 | Irx1 | 2772020 | 2772021 |
| cg10170045 | 3.019447718 | 0.000956208 | 0.097579967 | 368 | Nfix | 20336243 | 20336244 |
| cg12477370 | 3.016695048 | 0.000962288 | 0.097934292 | 369 | | | |
| cg12385032 | 3.014576037 | 0.000966994 | 0.098039455 | 370 | Bcas3 | 70788412 | 70788413 |
| cg22459894 | 3.013881405 | 0.000968542 | 0.098039455 | 371 | Poll | 176584945 | 176584946 |
| cg25471194 | 3.01192228 | 0.000972921 | 0.098197313 | 372 | Pbx3 | 5303776 | 5303777 |
| cg13325480 | 3.009701567 | 0.000977909 | 0.098197313 | 373 | Slc14a2 | 67895147 | 67895148 |
| cg02379339 | 3.008940773 | 0.000979624 | 0.098197313 | 374 | Myog | 33212180 | 33212181 |
| cg19972243 | 3.008525329 | 0.000980561 | 0.098197313 | 375 | Rragc | 138346303 | 138346304 |
| cg13087967 | 3.005593292 | 0.000987204 | 0.09859958 | 376 | Ranbp17 | 14425416 | 14425417 |
| cg00499875 | 3.001318925 | 0.000996968 | 0.099310673 | 377 | | | |

*The genomic coordinates for the CpGs in Table 2 are based on the Peromyscus maniculatus bairdii reference genome (NCBI RefSeq: GCF_003704035.1_HU_Pman_2.1.3, annotation release 100, updated Mar. 5, 2019), using the annotations provided by the Mammalian Methylation Consortium (github.com/shorvath/MammalianMethylationConsortium) and described in Horvath et al., 2021 [15]. See also Lu et al. 2023 [17] and Arneson et al. 2022 [42]. Corresponding CpGs in other organisms can be found in the above referenced resources. The genomic region sequences reflect consensus sequences from corresponding genomic regions in various organisms, including cat, cattle, dog, dolphin, elephant, horse, human, Peromyscus maniculatus bairdi, and Peromyscus leucopus. Exemplary oligonucleotides for determining the methylation status of the CpGs shown in Table 2 as reflected in the consensus sequences of SEQ ID NOS: 1-377 are SEQ ID NOS: 378-754, respectively.

### Rhythm of Methylation at Birth Resets in September

Subsequently, we normalized the average methylation values of the CpGs that exhibited circannual methylation by the maximum observed in any month to express methylation levels as a percentage. This analysis indicated that circannual methylation aligned with the solstice in June and December, and with the equinox in March and particularly in September (FIGS. 3A-3B and 4A-4F). Among the significantly differentially methylated CpGs (P<0.05, FDR<0.1), the vast majority (61%, 229 of 377) exhibited lowest methylation, while 10% (37 of 377) exhibited highest methylation in September. This preference of differential methylation in September-born animals highly exceeds the number of CpGs that are expected to be differentially methylated if this had occurred randomly throughout the year (P<0.001, chi sq. test). In 111 (29%) genes, the methylation pattern did not change direction in September (FIG. 5A). The strong preference for demethylation or hypermethylation in September is aligned to with the circannual pattern of breeding at which birth in September imposes the strongest memory of birth season and implies that the circannual methylation clock also resets during the autumn equinox.

When males (n=46) and females (n=50) were analyzed independently, we discovered that females displayed more pronounced cyclical methylation patterns compared to males (FIG. 5B) (Huynh-Dam et al. [41] at Additional file 2: Table S2 and S3). This observation, consistently with the analysis of breeding profiles, further supports that the females are more sensitive than the males in the effects of birth season. In the females, 3 CpGs exhibited significance for P<0.05 and FDR<0.1, but none in the males. When more relaxed criteria were used (P<0.05, FDR<0.2), 186 CpGs exhibited season of birth-dependent methylation in females but none in the males (Additional file 2: Table S2 and S3). The highly significant female-specific CpGs corresponded to genes Ypel3, Rnf220, and Nuf2 (FIG. 5C). Nuf2 is involved in kinetochore assembly [22], while Ypel3 and Rnf220 are involved in nervous system development [23, 24].

Comparison between monogamous and polygamous species indicated that circannual changes in methylation at birth were more prominent in the polygamous species (FIGS. 5D and 5E) (Huynh-Dam et al. [41] at Additional file 2: Table S4 and S5). In the former, 23 CpGs as opposed to none in the latter fitted sinusoidal modeling (P<0.01, FDR<0.2).

### Season-Specific Methylation and Biological Processes

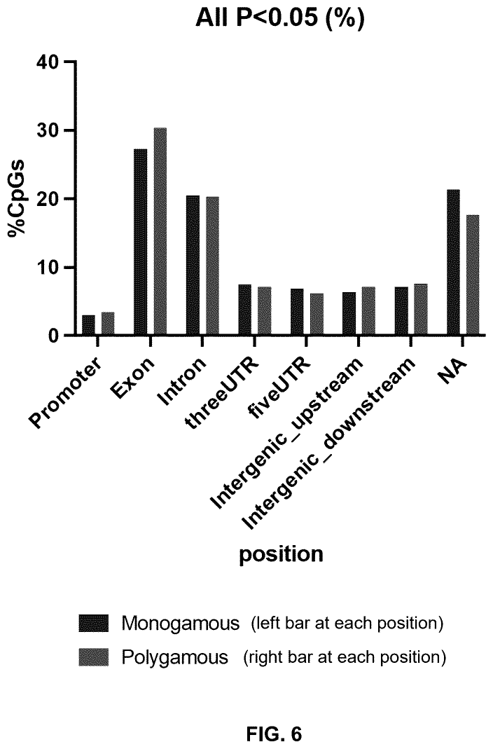

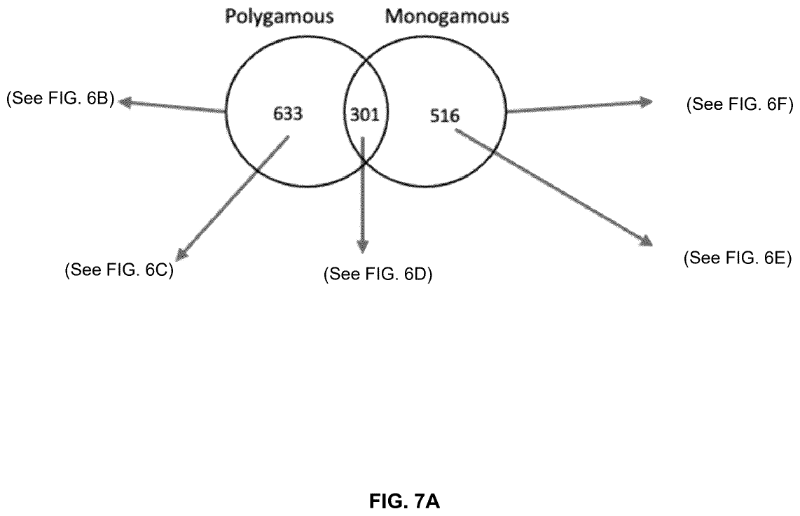

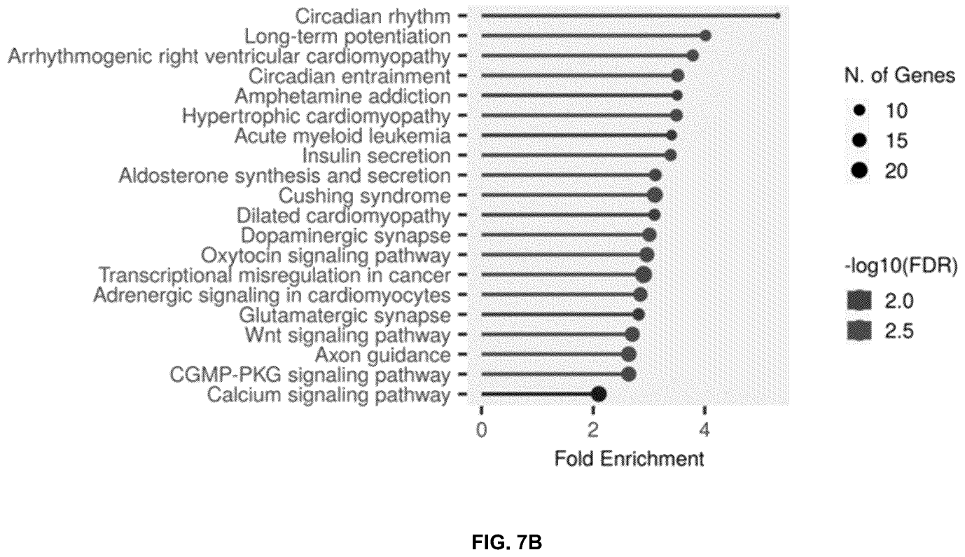

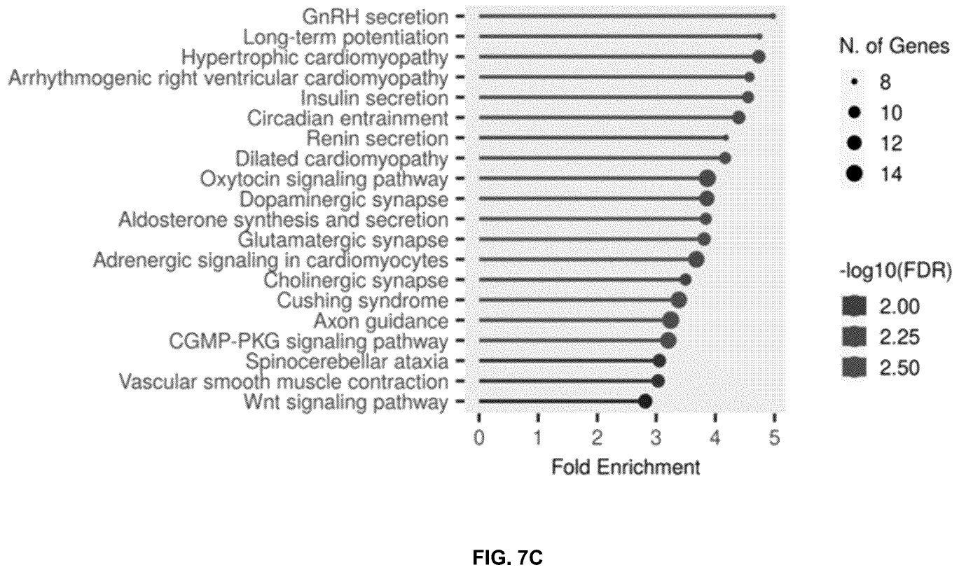

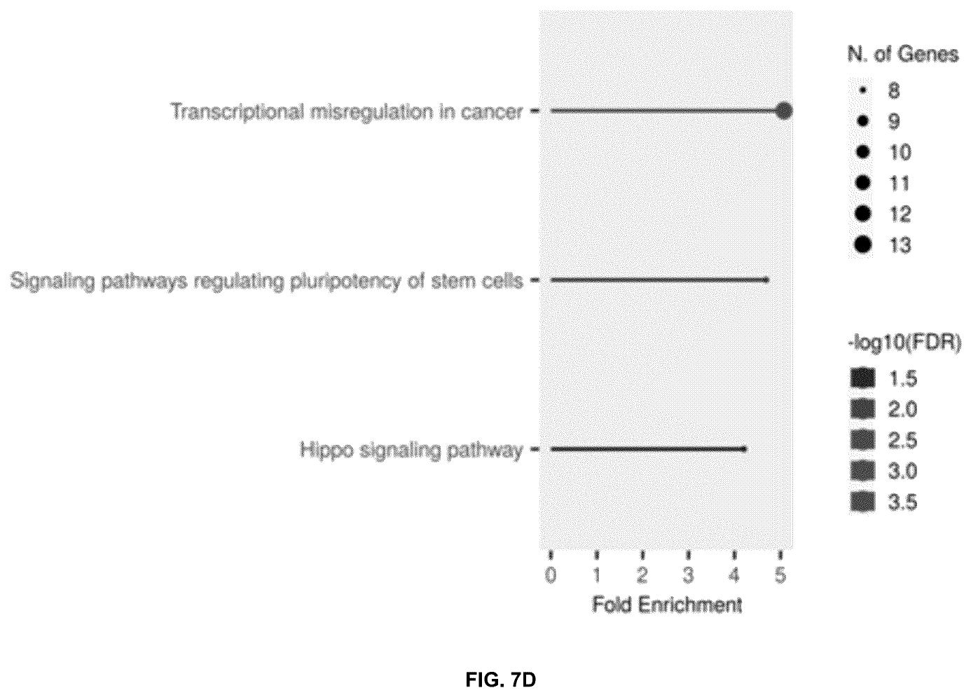

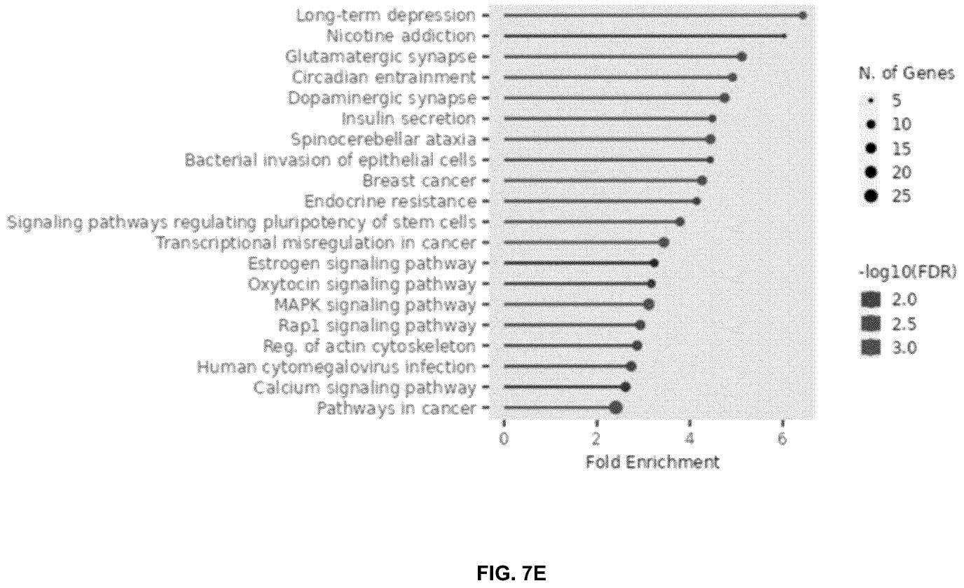

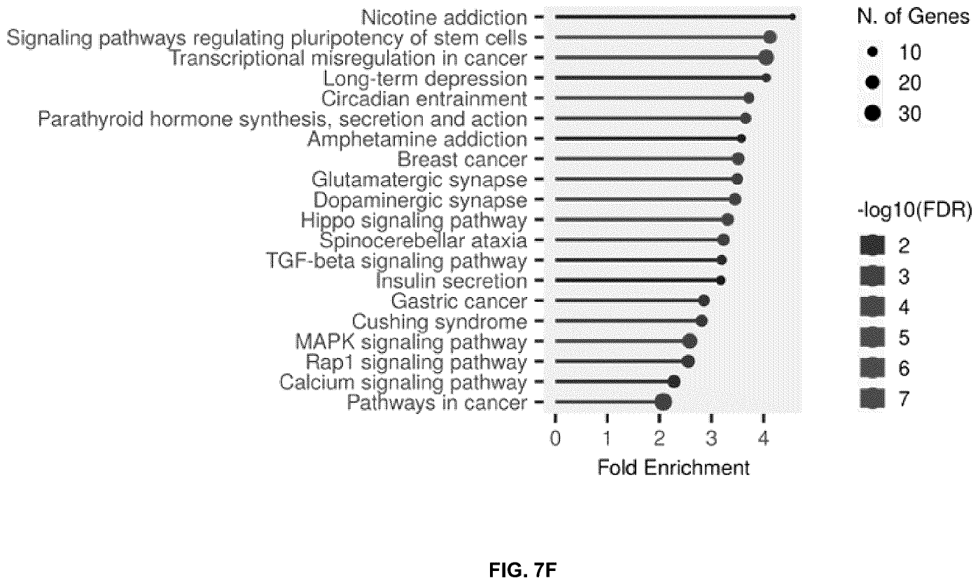

Subsequently, we explored if overlapping patterns of methylation exist in relation to their birth season between the species exhibiting a tendency for monogamous or polygamous behavior. Our analysis was controlled for age, sex, and species. To eliminate confounding effects of collection season on methylation, animals sampled in summer (n=11) were excluded, resulting in 85 samples included in this analysis. The gene ID, location, and position of the genes (using mouse genome references) methylated season-dependently are indicated in FIG. 6 and Huynh-Dam et al. [41] at Additional file 2: Table S6. Most significantly, methylated gene in the polygamous species included Msl2 that encodes for a ubiquitin ligase involved in H4-K16 acetylation during X chromosome dosage compensation [25, 26]. Six3 homeobox gene was most significantly methylated according to season of birth, in monogamous species, and encodes for a protein that is involved in embryonic development [27]. In the polygamous species, the most significantly (FDR=0.003) affected function was related to the secretion of gonadotropin hormone-releasing hormone (GnRH) which is tightly linked to reproductive behavior [28] (FIGS. 7A-7F). This provides mechanistic foundation to the distinct effects of season in the breeding performance and the association with the estrus cycle regulation of the polygamous species. Of note are also the opposite predicted effects of season, in the monogamous (FDR=0.0009) and the polygamous (FDR=0.004) species, respectively, in long term depression and potentiation (FIGS. 7A-7F). These processes are inherently related to memory and learning and may also relate to the specific ability of the polygamous species, at which long term potentiation is predicted, to comprehend and respond accordingly to the seasonal changes [29-31]. In monogamous species, seasonal changes in methylation predicted interference with processes associated with nicotine addiction (FDR=0.017). Nicotine addiction and pair bonding share signaling modules [32, 33]. Thus, the specific association of nicotine addiction with differentially methylated genes in monogamous species may reflect the sensitization of their genome to respond to ques related to addiction, which also relates to the establishment and maintenance of pair bonds.

### Environmental Signals in Captivity Impact Methylation

These observations indicate that the methylome of Peromyscus is responsive to seasonal cues. Nevertheless, the maintenance of deer mice, for more than 40 years for some stocks, at the regulated environment of the animal facilities deprive animals from the effects of photoperiod and temperature that are recognized as the most prominent factors regulating seasonality. It is plausible that changes in humidity, the presence of pollen in the air, or odors from the animal handling personnel can be sensed by deer mice and inform about the season. To test this hypothesis, we postulated that birth at individually ventilated cages (IVCs) may reduce such signals and alter the profiles of methylation as compared to those of animals that are born in conventional cages. Thus, we evaluated the methylation profile of Msl2 in cohorts of winter-born P. maniculatus and P. californicus, that were born either at conventional cages or IVCs. Msl2 exhibited season of birth-dependent methylation that deferred in polygamous and monogamous species. As shown in Huynh-Dam et al. [41] at FIG. 9, birth in winter at IVCs enhanced the methylation of Msl2 (cg11950309) in the former but not in the latter, towards a pattern that was more typical for the summer-born animals (P=0.01, chi-squared test). This observation shows that environmental cues in winter, rather than in summer, are sensed by polygamous P. maniculatus but not monogamous P. californicus, in the conventional cages, and not in the IVCs, and reduce Msl2 methylation at cg11950309. Furthermore, two other CpGs located in the close vicinity of cg11950309 were not impacted by birth at IVCs, further supporting the sensitivity of this CpG to environmental cues (Huynh-Dam et al. [41] at FIG. 9). Since both humidity and pollen are lower in South Carolina in winter where the animals are, we postulate that either their reduction in winter months or odors from the animal personnel at this period trigger these season-specific methylation changes in non-monogamous deer mice.

### Discussion

Our results suggest that circannual patterns exist in Peromyscus stocks are imposed upon birth and influence breeding performance and methylation. While the causality between reproduction profiles and methylation signatures remains elusive, both suggest that birth season is a modifier of animals' cellular and reproductive physiology. Noteworthy, methylation data were retrieved from tail analysis implying that the mechanism(s) linking epigenetic regulation and reproductive performance may be conserved across different tissues. In that case, changes in the epigenome detected in tails may reflect more widespread changes occurring at different tissues with a direct role in reproductive physiology, such as the gonads, hypothalamus, and the pituitary. Therefore, the observed patterns in tail methylation may represent global methylation changes, highlighting a systemic adaptation to circannual rhythms that affects the organism as a whole.

Understanding of birth season has also been reported in human populations and was associated with circannual patterns in hormone levels and an increased lifespan of individuals that were born in winter [34, 35]. The striking synchronization of both the methylation and of the breeding profiles with the equinox in deer mice, especially in September at which the highest fitness was identified following application of SINE and 12-knots GAM modeling, points to a pivotal role of this period in resetting the circannual clock. Following that time point, progressively fitness decreases reaching a trough before the solstice and then it increases again until the next equinox.

The circannual rhythms recorded here likely reflect evolutionary adaptations by which animals can be impacted by the season and adjust their reproduction accordingly. The period between the autumn and the spring equinox marks the winter period at which the environmental conditions are unfavorable. An intriguing possibility is that animals born at this period retain an elusive memory of these conditions and favor reproduction in the summer. This was confirmed when absolute numbers of the offspring of winter-born (October-March) animals were compared to those of summer-born (April-September) animals in born winter or summer. The summer-born animals, especially those born from May to July when the conditions are mostly favorable, breed throughout the year without strongly favoring particular seasons for littering. These effects were more prominent in the females than in the males and were also reflected to the season of birth methylation profiles that showed better fitness in a sinusoidal model in the former than in the latter. In humans, women also exhibited higher sensitivity than men to the effects of season, implying the persistence of an evolutionary adaptation according to which non-seasonal breeders, such as Peromyscus, may retain sex-dependent differences in the effects of season [36]. Evidence for memory of cold environment, and a subsequent adaptive response in metabolism, was also recently reported in a study involving laboratory mice that demonstrated the role of the hypothalamus in this process [37].

The evolutionary relevance of the memory of birth is also supported by the observation that in monogamous species, seasonal effects are likely reduced in both the circannual methylation and in the effects of parental birth month in reproduction. If indeed the breeding strategy is causally associated and is not coincidental due to the phylogenetic relations, an intriguing explanation is that monogamy shields animals from environmental signals that influence physiology and behavior. It is noted, however, that the weakest association of breeding with birth season, especially in P. californicus and P. eremicus compared to P. leucopus and P. maniculatus, may not reflect a consequence of monogamy per se but rather a phenotypic characteristic of this clade of Peromyscus, since the former two and the latter two Peromyscus species are evolutionary more related than the other two [10]. This is supported also by the moderate correlation of both the monogamous P. polionotus and of the high-altitude P. maniculatus bairdii between birth season and preference of season for reproduction.

An intriguing finding of this study is the persistence of circannual rhythms in animals that are maintained in controlled animal facilities. The fact that the Peromyscus stocks used here are maintained for several decades as closed colonies at controlled environments suggests that the circannual rhythm is imposed at birth, by mechanisms that are independent of photoperiod and temperature that constitute the most highly appreciated stimuli controlling seasonality in mammals and other animals. Whether this regulation reflects the operation of an epigenetic clock that was imposed prior to the captivation of the original breeders from the wild and persisted thereafter, or they reflect environmental responses triggered by geomagnetic signals, or signals originating from the animal-caring personnel through odors or other stimuli, remains to be established. The fact however that the analysis involved records that range from 35 to 60 years old and persisted thereafter argues against the persistence of a rhythm that originated in the ancestral populations and implies that external signals are periodically inflicted and are received by the animals in captivity to strengthen their memory of birth season. This notion is further supported by the observation that birth at IVCs abolishes the methylation profile that is imposed when birth occurs in conventional cages, at least in P. maniculatus.

The small number of stocks (only 3 monogamous and only 3 polygamous) constitutes a formal limitation of our study, and our findings should be interpreted with caution. FDR was not considered in the comparative CpG analysis between monogamous and polygamous stocks, but only P values; nonetheless, the recorded differences were consistent with those unveiled when fitness in a sinusoidal model was tested at which FDR was considered. Furthermore, it remains formally plausible that the recorded differences reflect differences in the two corresponding clades of Peromyscus, involving LL, BW, SM2 and PO in one, and IS and EP in the other [10]. In addition to these limitations, the relative ambiguity in the breeding strategies during the earlier periods may have potentially impacted the stocks' reproductive behavior. Nevertheless, the observation that constant breeding patterns were maintained throughout the observation period suggests that potential variation in the breeding strategies should not have impacted considerably the reproductive dynamics of the stocks in captivity.

### Conclusions

Our findings, besides their ecological and physiological implications, suggest that animals at controlled facilities remain vulnerable to the effects of season which may function as modulators of physiological responses in experimental studies. To that end, rodents such as Peromyscus, and probably other species that are used as experimental models, have modified physiology and epigenetic landscape depending on their season of birth, which may modulate the results of experimental studies. Therefore, the effects of season should be studied in experimental models, and probably season may be considered a modifier when experimental findings are projected to human populations.

### Methods

### Breeding Program and Records

Breeding of deer mice is performed in accordance with Institutional guidelines throughout the years of the operation of the PGSC. Detailed records on animal husbandry are available for more than 35 years and the details are presented herein. Until 2023, all stocks, with the exception of SM2, were kept at conventional, not individually ventilated cages at 25° C. in a 16-h light/8-h dark cycle throughout the year, and received standard chow diet and water ad libitum. In 2023, stocks were progressively transferred to IVCs at which they are maintained thereafter. For SM2, the transfer was implemented earlier due to poor breeding performance. Currently approved IACUC is 2644-101795-042823. Animal husbandry was attained by the animal facility personnel, while the establishment of breeding pairs and weaning was performed by the colony manager. No evidence of periodic changes in the animal care personnel were noted. Stock maintenance is performed by breeding pairs that are historically established by animals typically older than 2 months old, selected randomly by the colony manager. Random assignment to breeding pairs involved no phenotypic selection of mating partners according to their characteristics and whenever feasible, avoidance of breeding of animals that shared ancestors for at least 2 generations. Breeders usually are maintained for more than one year, usually 1.5 to 2 years, before their replacement by other, younger breeders. Dates of birth and the pup numbers and sex at weaning are recorded manually and electronically. Since the exact number of breeders that were historically established, and the time at which the breeding pairs were set are not known, the assessment of the total number of mice produced may had been misleading when the breeding patterns are examined. Therefore, in modeling circannual breeding performance, we divided the breeding records in 5-year intervals, and from all productive litters, we considered only the litters that were produced within 12 months since the production of the first litter (including the first litter). The resulting litter numbers were expressed as the (%) of the total offspring that were produced within this 12-month period and were averaged between the different 5-year interval for each stock. This approach allows inclusion of each breeding pair (and of its litters) only once, thus does create bias toward specific breeders. It also eliminates the possibility that offspring production is favored at particular seasons just because of the different numbers of breeders that were set or because they were maintained for different periods. In addition, not only the reduced breeding performance due to aging was eliminated as a variable, but also the production of litters at specific time following birth. Furthermore, since the average litter sizes differ in the different stocks and because at different periods of the PGSC operation the overall size of the colonies could been drastically different (due to varied demand for animals), offspring number were normalized according to the total number of offspring that were produced within this 12-month period. Finally, to consider that changes in the breeding behavior of deer mice might have had occurred since the original establishment of the stocks, the breeding data, instead of being expressed as cumulative aggregates, they were clustered together in 5-year intervals, and the average values of these intervals were considered. To that end, the 5-year averages represented the unit of observation in modeling circannual breeding patterns. Proportions of offspring born in each month were calculated and plotted by parent birth month and stock, allowing us to examine how the birth month of the sire or dam influenced seasonal patterns in reproduction. These analyses were descriptive and involved no formal hypothesis testing. Standard errors of the mean (SEM) were used for visualization of variability across years.

### Analysis of Breeding Patterns

The database of Peromyscus colonies was maintained in Microsoft Access and exported to excel files for bioinformatic analysis. Month of litters' birth for dams and sires of each mating cage was calculated for 1 year from first time of delivery, using R (R Core Team, 2024) and RStudio (Rstudio Team, 2024). For circannual analysis, the breeding patterns were subjected to sinusoidal model (SINE) and generalized additive model (GAM) to unravel periodicity and trends in reproductive behavior (see above section for details on litters considered). For comparative analyses of breeding in summer vs winter, all offspring and litters were included (see text for details and Huynh-Dam et al. [41] at FIG. 4 and Additional file 1: FIG. S7). For SM2 and LL stocks, the initial 3-5 years period was omitted due to poor or inconsistent breeding. The analysis was conducted using Python 3.10 and Jupyter Notebook.

### Differential Methylation Analysis

The raw DNA methylation data were retrieved from previous publication [115], and only tail specimens were used. For circannual methylation analysis, specimens had age range of 1-35 months and included 16 IS, 16 PO, 16 LL, 10 SM2, 17 EP, 15 BW, and 6 PO×BW F1 hybrids. The same dataset was included in the comparative analysis of methylation in summer-born vs winter-born polygamous and monogamous animals, excluding 11 specimens at which sampling occurred in summer.

To investigate the relationship between seasonality of birth and DNA methylation profiles, we applied site-specific linear regression models to normalized β-values obtained from SeSAMe-normalized dataset. Each CpG site was analyzed separately across individuals.

The unit of observation was the individual mouse at each CpG locus. Each row in the model corresponded to one methylation value for a specific site in a specific animal.

Our models controlled for known covariates that influence DNA methylation: Age, Sex, and SpeciesAbbreviation (used as a proxy for genetic stock). These variables were included as fixed effects in all models.

We did not include parental cage (i.e., breeder pair) as a random effect. Although parental cage identity was available, most cages contributed only 1-2 offspring, and birth dates varied within cages. This limited structure made inclusion of breeder pair statistically unstable and potentially confounding. We therefore focused on individual-level modeling while adjusting for key biological covariates.

For the seasonal model, we tested whether birth in winter versus summer predicted methylation:

### Null Model:

$${ProtMet}_{ij} = {β}_{0} + {β}_{1} \cdot {Age}_{i} + {β}_{2} \cdot {Sex}_{i} + {β}_{3} \cdot {Species}_{i} + {ε}_{ij}$$

### Full Model:

$${ProtMet}_{ij} = {β}_{0} + {β}_{1} \cdot {Age}_{i} + {β}_{2} \cdot {Sex}_{i} + {β}_{3} \cdot {Species}_{i} + {β}_{4} \cdot {BirthSeason}_{i} + {ε}_{ij}$$

For the circannual model, we modeled birth month as a cyclical variable using sine and cosine transformations. This approach allows for capturing continuous seasonal variation across the year and avoids artificial cutoffs between months like December and January. This method is well-established for modeling periodic variables in epidemiological and environmental data [18].

### Full Model:

$${ProtMet}_{ij} = {β}_{0} + {β}_{1} \cdot {Age}_{i} + {β}_{2} \cdot {Sex}_{i} + {β}_{3} \cdot {Species}_{i} + {β}_{4} \cdot sin ( 2 π \cdot {Month}_{i} / 12 ) + {β}_{5} \cdot cos ( 2 π \cdot {Month}_{i} / 12 ) + {ε}_{ij}$$

Likelihood ratio tests (LRTs) were used to compare each null and full model pair, and significance was reported as −Log 10(P). To account for multiple hypothesis testing across the methylome, p-values were corrected using the Benjamini-Hochberg False Discovery Rate (FDR) method [38].

All analyses were conducted in R (v4.3.1), and the complete reproducible analysis pipeline—including model fitting, nested comparisons, and output visualization—is publicly available on the world wide web at github.com/KimTuyenHuynhDam/circanual_seasonality_breeding_pero.

### GO Analysis

Gene Ontology (GO) analysis was performed using ShinyGO 0.77 (bioinformatics.sdstate.edu/go/). The top enrichment was selected by FDR, sorted by fold enrichment. For functional enrichment analysis, differentially methylated CpG sites identified through this approach were mapped to their respective genes, forming the foreground dataset. The background dataset consisted of all CpG sites that passed quality control within the SeSAMe dataset. Since GO tools assess enrichment at the gene level rather than the CpG level, we aggregated CpG sites to their corresponding genes to mitigate biases arising from variations in CpG density across different genomic regions [39]. Figures were produced by using Prism GraphPad Software, LLC (San Diego CA).

### Estrus Cytology

The vaginal smears were collected and stained every day for the period of continuous 9-14 days using the protocol described previously [40]. The observation of smears collected daily informed about the length of the estrus cycle by recording the days until recurrent cellular patterns were observed.

### Methylation of a Single CpG Study

The sequence of each CpG was retrieved according to their annotation and aligned on NCBI (blast.ncbi.nlm.nih.gov/) to get their exact sequence and confirmed the prediction of CpG and its related gene. The truncated genome sequence containing CpGs was put in MethPrimer (urogene.org/cgi-bin/methprimer/methprimer.cgi) to get the primers for methylated CpG analysis.

The tails of mice in IVCs or conventional cages were collected at weaning. DNA from collected tails was isolated using Quick-DNA Microprep Plus Kit (Zymo Research) and then bisulfite treated using EZ DNA Methylation-Lightning Kit (Zymo Research) according to the supplied protocol. The sequence of Msl2 (cg11950309) was amplified by PCR using 100 ng bisulfite-treated DNA, ZymoTaq PreMix (Zymo Research), primer 1 (forward) (5′-AGTTTTAGGAAAAGAAAGGATGTAAATGTG (SEQ ID NO:755)) and primer 2 (reverse) (5′ CAAATTAAACCAACAAAACCATAACTACC (SEQ ID NO:756)) before sending to Sanger Sequencing (Eton Bioscience). The resulting sequences were read on FinchTV software, and the methylated peaks were analyzed by ImageJ software. The graphs of analyzed results were produced using RStudio.

### Statistical Analysis

Data were analyzed by Wilcoxon test, paired and unpaired t test, or chi-squared test, as indicated in the text and graphs. P values are indicated in the graphs.

### Abbreviations

PGSC: Peromyscus Genetic Stock Center. PO: P. polionotus. BW: P. maniculatus bairdii. LL: P. leucopus. SM2: P. maniculatus sonoriensis. IS: P. californicus. EP: P. eremicus. GAM: Generalized additive model. SINE: Sinusoidal. SEM: Standard error of the mean. LRT: Likelihood ratio test. GO: Gene Ontology.

### REFERENCES

1. Fisher R. The genetical theory of natural selection. 1958.
2. Varpe Ø. Life history adaptations to seasonality. Integr Comp Biol. 2017; 57(5):943-60.
3. De Jong S, Neeleman M, Luykx J J, ten Berg M J, Strengman E, Den Breeijen H H, et al. Seasonal changes in gene expression represent cell-type composition in whole blood. Hum Mol Genet. 2014; 23(10):2721-8.
4. Luykx J, Bakker S, Van Geloven N, Eijkemans M, Horvath S, Lentjes E, et al. Seasonal variation of serotonin turnover in human cerebrospinal fluid, depressive symptoms and the role of the 5-HTTLPR. Translational Psychiatry. 2013; 3(10):e311-e.
5. Luykx J J, Bakker S C, Lentjes E, Boks M P, van Geloven N, Eijkemans M J, et al. Season of sampling and season of birth influence serotonin metabolite levels in human cerebrospinal fluid. PLoS ONE. 2012; 7(2): e30497.
6. Tendler A, Bar A, Mendelsohn-Cohen N, Karin O, Korem Kohanim Y, Maimon L, et al. Hormone seasonality in medical records suggests circannual endocrine circuits. Proc Natl Acad Sci USA. 2021; 118(7).
7. Doblhammer G, Vaupel J W. Lifespan depends on month of birth. Proc Natl Acad Sci USA. 2001; 98(5):2934-9.
8. Drickamer L C. Seasonal-variation in fertility, fecundity and litter sex-ratio in laboratory and wild stocks of house mice (Mus-domesticus). Lab Anim Sci. 1990; 40(3):284-8.
9. Steel L C E, Tam S K E, Brown L A, Foster R G, Peirson S N. Light sampling behaviour regulates circadian entrainment in mice. BMC Biol. 2024; 22(1):208.
10. Turner L M, Young A R, Römpler H, Schöneberg T, Phelps S M, Hoekstra H E. Monogamy evolves through multiple mechanisms: evidence from V1aR in deer mice. Mol Biol Evol. 2010; 27(6):1269-78.
11. Wood S N. Generalized additive models: an introduction with R: chapman and hall/CRC; 2017.
12. Jašarević E, Bailey D H, Crossland J P, Dawson W D, Szalai G, Ellersieck M R, et al. Evolution of monogamy, paternal investment, and female life history in Peromyscus. J Comp Psychol. 2013; 127(1):91.
13. Lee T, McClintock M. Female rats in a laboratory display seasonal variation in fecundity. Reproduction. 1986; 77(1):51-9.
14. White F, Wettemann R, Looper M, Prado T, Morgan G. Seasonal effects on estrous behavior and time of ovulation in nonlactating beef cows. J Anim Sci. 2002; 80(12):3053-9.
15. Horvath S, Haghani A, Zoller J A, Naderi A, Soltanmohammadi E, Farmaki E, et al. Methylation studies in Peromyscus: aging, altitude adaptation, and monogamy. Geroscience. 2022:1-15.
16. Haghani A, Li C Z, Robeck T R, Zhang J, Lu A T, Ablaeva J, et al. DNA methylation networks underlying mammalian traits. Science. 2023; 381(6658):eabg5693.
17. Lu A T, Fei Z, Haghani A, Robeck T R, Zoller J, Li C, et al. Universal DNA methylation age across mammalian tissues. Nature aging. 2023; 3(9):1144-66.
18. Stolwijk A, Straatman H, Zielhuis G. Studying seasonality by using sine and cosine functions in regression analysis. J Epidemiol Community Health. 1999; 53(4):235-8.
19. Garijo A M A, Lacambra J M. The likelihood ratio test of common factors under non-ideal conditions. Investigaciones Regionales Journal of Regional Research. 2011(21):37-52.
20. Schweizer F E, Dresbach T, DeBello W M, O'Connor V, Augustine G J, Betz H. Regulation of neurotransmitter release kinetics by NSF. Science. 1998; 279(5354):1203-6.
21. Gao Y, Khan Y A, Mo W, White K I, Perkins M, Pfuetzner R A, et al. Sensory deficit screen identifies nsf mutation that differentially affects SNARE recycling and quality control. Cell reports. 2023; 42(4).
22. DeLuca J G, Dong Y, Hergert P, Strauss J, Hickey J M, Salmon E, et al. Hec1 and nuf2 are core components of the kinetochore outer plate essential for organizing microtubule attachment sites. Mol Biol Cell. 2005; 16(2):519-31.
23. Blanco-Sánchez B, Clément A, Stednitz S J, Kyle J, Peirce J L, McFadden M, et al. yippee like 3 (ypel3) is a novel gene required for myelinating and perineurial glia development. PLoS Genet. 2020; 16(6): e1008841.
24. Li Y, Wan L P, Song N-N, Ding Y-Q, Zhao S, Niu J, et al. RNF220-mediated K63-linked polyubiquitination stabilizes Olig proteins during oligodendroglial development and myelination. Science advances. 2024; 10(6):eadk3931.
25. Smith E R, Cayrou C, Huang R, Lane W S, Côté J, Lucchesi J C. A human protein complex homologous to the Drosophila MSL complex is responsible for the majority of histone H4 acetylation at lysine 16. Mol Cell Biol. 2005; 25(21):9175-88.
26. Wu L, Zee B M, Wang Y, Garcia B A, Dou Y. The RING finger protein MSL2 in the MOF complex is an E3 ubiquitin ligase for H2B K34 and is involved in crosstalk with H3 K4 and K79 methylation. Mol Cell. 2011; 43(1):132-44.
27. Meurer L, Ferdman L, Belcher B, Camarata T. The SIX family of transcription factors: common themes integrating developmental and cancer biology. Frontiers Cell and Developmental Biology. 2021; 9: 707854.
28. Casteel C O, Singh G. Physiology, gonadotropin-releasing hormone. StatPearls. Treasure Island (FL) 2025.
29. Rygvold T W, Hatlestad-Hall C, Elvsåshagen T, Moberget T, Andersson S. Long term potentiation-like neural plasticity and performance-based memory function. Neurobiol Learn Mem. 2022; 196: 107696.
30. Monday H R, Younts T J, Castillo P E. Long-term plasticity of neurotransmitter release: emerging mechanisms and contributions to brain function and disease. Annu Rev Neurosci. 2018; 41(1):299-322.
31. Zucker R S. Calcium- and activity-dependent synaptic plasticity. Curr Opin Neurobiol. 1999; 9(3):305-13.
32. Resendez S L, Keyes P C, Day J J, Hambro C, Austin C J, Maina F K, et al. Dopamine and opioid systems interact within the nucleus accumbens to maintain monogamous pair bonds. elife. 2016; 5:e15325.
33. Zou Z, Song H, Zhang Y, Zhang X. Romantic love vs. drug addiction may inspire a new treatment for addiction. Frontiers in psychology. 2016; 7:187913.
34. Doblhammer G, Vaupel J W. Lifespan depends on month of birth. Proc Natl Acad Sci. 2001; 98(5):2934-9.
35. Tendler A, Bar A, Mendelsohn-Cohen N, Karin O, Korem Kohanim Y, Maimon L, et al. Hormone seasonality in medical records suggests circannual endocrine circuits. Proc Natl Acad Sci. 2021; 118(7): e2003926118.
36. Bryson A, Blanchflower D G. Seasonality and the female happiness paradox. Quality and Quantity: international journal of methodology. 2023.
37. Munoz Zamora A, Douglas A, Conway P B, Urrieta E, Moniz T, O'Leary J D, et al. Cold memories control whole-body thermoregulatory responses. Nature. 2025.
38. Benjamini Y, Hochberg Y. Controlling the False Discovery Rate—a Practical and Powerful Approach to Multiple Testing. J R Stat Soc B. 1995; 57(1):289-300.
39. Geeleher P, Hartnett L, Egan L J, Golden A, Raja Ali R A, Seoighe C. Gene-set analysis is severely biased when applied to genome-wide methylation data. Bioinformatics. 2013; 29(15):1851-7.
40. McLean A C, Valenzuela N, Fai S, Bennett S A. Performing vaginal lavage, crystal violet staining, and vaginal cytological evaluation for mouse estrous cycle staging identification. J Visualized Experiments: JoVE. 2012; 67:4389.
41. Huynh-Dam K T, Jaeger C, Naderi A, Bayram M F, Crossland J P, Kaza V, Barlow S, Chatzistamou I, Long A, Horvath S, Kiaris H. Circannual breeding and methylation are impacted by the equinox in Peromyscus. BMC Biol. 2025 Jun. 2; 23(1):149.
42. Arneson A, Haghani A, Thompson M J, Pellegrini M, Kwon S B, Vu H, Maciejewski E, Yao M, Li C Z, Lu A T, Morselli M, Rubbi L, Barnes B, Hansen K D, Zhou W, Breeze C E, Ernst J, Horvath S. A mammalian methylation array for profiling methylation levels at conserved sequences. Nat Commun. 2022 Feb. 10; 13(1):783.

What is claimed is:

1. A method comprising determining a methylation level of any one or more CpG sites in each of 10 or more genomic regions of a genome of a subject, wherein the genomic regions have sequences of any 10 or more of SEQ ID NOS:1-377 or sequences at least 80% identical thereto.

2. The method of claim 1, wherein the genomic regions have sequences of any 10 or more of SEQ ID NOS:1-25 or sequences at least 80% identical thereto.

3. The method of claim 1, comprising determining a methylation level of any one or more CpG sites in each of 25 or more genomic regions of the genome of the subject, wherein the genomic regions have sequences of each of SEQ ID NOS:1-25 or sequences at least 80% identical thereto.

4. The method of claim 1, comprising determining a methylation level of any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 10 of SEQ ID NOS:1-25 or sequences at least 80% identical thereto.

5. The method of claim 1, comprising determining a methylation level of any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 15 of SEQ ID NOS:1-25 or sequences at least 80% identical thereto.

6. The method of claim 1, comprising determining a methylation level of any one or more CpG sites in each of 100 or more genomic regions of the genome of the subject, wherein the genomic regions have sequences of any 100 or more of SEQ ID NOS:1-377 or sequences at least 80% identical thereto, with the proviso that the genomic regions have sequences of at least 25 of SEQ ID NOS:1-50 or sequences at least 80% identical thereto.

7. The method of claim 1, wherein a methylation level is determined for no more than 30,000 CpG sites in the genome of the subject.

8. The method of claim 1, wherein the determining comprises:
treating genomic DNA from the subject with bisulfite to generate bisulfite-treated genomic DNA;
amplifying the bisulfite-treated genomic DNA using primers that amplify portions of the bisulfite-treated genomic DNA comprising the genomic regions; and
measuring the methylation level of the one or more CpG sites in each of the genomic regions.

9. The method of claim 8, wherein the portion of the bisulfite-treated genomic DNA has a length less than 1000 bases.

10. The method of claim 1, wherein the determining comprises:
treating genomic DNA from the subject with bisulfite to generate bisulfite-treated genomic DNA;
amplifying the bisulfite-treated genomic DNA using primers specific for portions of the bisulfite-treated genomic DNA comprising the genomic regions; and
measuring the methylation level of the one or more CpG sites in each of the genomic regions.

11. The method of claim 10, wherein the portion of the bisulfite-treated genomic DNA has a length less than 1000 bases.

12. The method of claim 1, wherein the methylation level is measured by methylation-specific PCR, quantitative methylation-specific PCR, methylation-sensitive DNA restriction enzyme analysis, or bisulfite genomic sequencing PCR, or quantitative bisulfite pyrosequencing.

13. The method of claim 1, further comprising determining from the methylation level of the one or more CpG sites a birth period of the subject.

14. An array of immobilized oligonucleotides, wherein the oligonucleotides are configured to bind to amplicons of bisulfite modified genomic DNA, wherein the amplicons comprise bisulfite modified CpG sites corresponding to any one or more CpG sites in each of 10 or more genomic regions of a genome of a subject, wherein the genomic regions have sequences of any 10 or more of SEQ ID NOS:1-377 or sequences at least 80% identical thereto.

15. The array of claim 14, wherein the oligonucleotides are configured to bind to amplicons comprising bisulfite modified CpG sites corresponding to any one or more CpG sites in each of 10 or more genomic regions of the genome of the subject, wherein the genomic regions have sequences of any 10 or more of SEQ ID NOS:1-25 or sequences at least 80% identical thereto.

16. The array of claim 14, wherein the oligonucleotides are configured to bind to amplicons comprising bisulfite modified CpG sites corresponding to any one or more CpG sites in each of 10 or more genomic regions of the genome of the subject, wherein the genomic regions have sequences of each of SEQ ID NOS:1-25 or sequences at least 80% identical thereto.

17. The array of claim 14, wherein the oligonucleotides are configured to bind to amplicons comprising bisulfite modified CpG sites corresponding to any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 10 of SEQ ID NOS:1-25 or sequences at least 80% identical thereto.

18. The array of claim 14, wherein the oligonucleotides are configured to bind to amplicons comprising bisulfite modified CpG sites corresponding to any one or more CpG sites in each of 50 or more genomic regions of the genome of the subject, wherein the genomic regions have sequences of any 50 or more of SEQ ID NOS:1-377 or sequences at least 80% identical thereto, with the proviso that the genomic regions have sequences of at least 15 of SEQ ID NOS:1-25 or sequences at least 80% identical thereto.

19. The array of claim 14, wherein the oligonucleotides are configured to bind to amplicons comprising bisulfite modified CpG sites corresponding to any one or more CpG sites in each of 100 or more genomic regions of the genome of the subject, wherein the genomic regions have sequences of any 100 or more of SEQ ID NOS:1-377 or sequences at least 80% identical thereto, with the proviso that the genomic regions have sequences of at least 25 of SEQ ID NOS:1-50 or sequences at least 80% identical thereto.

20. The array of claim 14, wherein the array comprises no more than 30,000 different oligonucleotides.
